SEQUENCE LISTING (105 sequences)

SEQ ID NO: 1 - DNA, 1586 bp, Porcine reproductive and respiratory syndrome virus
  CDS (175)..(234)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatcctctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgtccctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaagtctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacctaaacggacctatcgtcgtacagtacttctccgttaaggagagttggatccgccatttgaaactggcgggagaacccagctactctgggtttgaggacctcctcagaataagggttgagcctaacacgtcgccattggctgacaaggaagaaaaaattttccggtttggcagtcacaagtggtacggcgctctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 2 - DNA, 1149 bp, Porcine reproductive and respiratory syndrome virus
tctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgtccctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaagtctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacctaaacggacctatcgtcgtacagtacttctccgttaaggagagttggatccgccatttgaaactggcgggagaacccagctactctgggtttgaggacctcctcagaataagggttgagcctaacacgtcgccattggctgacaaggaagaaaaaattttccggtttggcagtcacaagtggtacggcgct

SEQ ID NO: 3 - protein, 20 aa, Porcine reproductive and respiratory syndrome virus
MEQKLISEEDLNRSMNSSGS

SEQ ID NO: 4 - DNA, 3968 bp, Porcine reproductive and respiratory syndrome virus
  CDS (175)..(201)
  CDS (3766)..(3789)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccgccgcgctttgtccgttcgtgaaacccggcaggccaaggagcacgaggttgccggccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcacagcggagggaatcaccctgaccaagtgcgcttagggaaaattatcagcctttgtcaggtgattgaggactgctgctgttcccagaacaaaaccaaccgggtcaccccggaggaggtcgcagcaaagattgacctgtacctccgtggtgcaacaaatcttgaagaatgcttggccaggcttgagaaagcgcgcccgccacgcgtaatcgacacctcctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaactactactaccgtgcgcaaggtgacgaagttcgtcaccgtgaaagactaaccgccgtgctctccaagttggaaaaggttgttcgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccaggacgagcccctggatttgtctgcttcctcacagactgaatatgaggcctctcccccagcaccgccgcagagcgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaaacatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggctcttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctaccctggcggtggttattctccgggtgattggggttttgcagcttttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgttgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcagtcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagctctccggtctggctactccaccacaaacctcgtccttggtgtaggggactttgcccagctgaatggattaaaaatcaggcaaatttccaagccttcagggctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 5 - DNA, 3583 bp, Porcine reproductive and respiratory syndrome virus
gctggaaagagagcaagaaaagcacgctcttgtgcgactgctacagtcgctggccgcgctttgtccgttcgtgaaacccggcaggccaaggagcacgaggttgccggccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcacagcggagggaatcaccctgaccaagtgcgcttagggaaaattatcagcctttgtcaggtgattgaggactgctgctgttcccagaacaaaaccaaccgggtcaccccggaggaggtcgcagcaaagattgacctgtacctccgtggtgcaacaaatcttgaagaatgcttggccaggcttgagaaagcgcgcccgccacgcgtaatcgacacctcctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaactactactaccgtgcgcaaggtgacgaagttcgtcaccgtgaaagactaaccgccgtgctctccaagttggaaaaggttgttcgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccaggacgagcccctggatttgtctgcttcctcacagactgaatatgaggcctctcccccagcaccgccgcagagcgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaaacatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggctcttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctaccctggcggtggttattctccgggtgattggggttttgcagcttttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgttgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcagtcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagctctccggtctggctactccaccacaaacctcgtccttggtgtaggggactttgcccagctgaatggattaaaaatcaggcaaatttccaagccttcaggg

SEQ ID NO: 6 - protein, 9 aa, Porcine reproductive and respiratory syndrome virus
MEQKLISEE

SEQ ID NO: 7 - DNA, 1049 bp, Porcine reproductive and respiratory syndrome virus
  CDS (175)..(870)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatagctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 8 - DNA, 611 bp, Porcine reproductive and respiratory syndrome virus
ggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatagctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgtcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaa

SEQ ID NO: 9 - protein, 232 aa, Porcine reproductive and respiratory syndrome virus
MEQKLISEEDLNRSMNSSGSGAFRTRKPSLNTVNVIGSSMGSGGVFTIDGKVKCVTAAHVLTGNSARVSGVGFNQMLDFDVKGDFAIADCPNWQGAAPKTQFCTDGWTGRAYWLTSSGVEPGVIGKGFAFCFTACGDSGSPVITEAGELVGVHTGSNKQGGGIVTRPSGQFCNVAPIKLSELSEFFAGPKVPLGDVKVGSHIIKDISEVPSDLCALLAAKPELELEHHHHHH

SEQ ID NO: 10 - DNA, 2588 bp, Porcine reproductive and respiratory syndrome virus
  CDS (175)..(234)
  CDS (2386)..(2409)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcacagcggagggaatcaccctgaccaagtgcgcttagggaaaattatcagcctttgtcaggtgattgaggactgctgctgttcccagaacaaaaccaaccgggtcaccccggaggaggtcgcagcaaagattgacctgtacctccgtggtgcaacaaatcttgaagaatgcttggccaggcttgagaaagcgcgcccgccacgcgtaatcgacacctcctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaactactactaccgtgcgcaaggtgacgaagttcgtcaccgtgaaagactaaccgccgtgctctccaagttggaaaaggttgttcgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccaggacgagcccctggatttgtctgcttcctcacagactgaatatgaggcctctcccccagcaccgccgcagagcgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaaacatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 11 - DNA, 2151 bp, Porcine reproductive and respiratory syndrome virus
gctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcacagcggagggaatcaccctgaccaagtgcgcttagggaaaattatcagcctttgtcaggtgattgaggactgctgctgttcccagaacaaaaccaaccgggtcaccccggaggaggtcgcagcaaagattgacctgtacctccgtggtgcaacaaatcttgaagaatgcttggccaggcttgagaaagcgcgcccgccacgcgtaatcgacacctcctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaactactactaccgtgcgcaaggtgacgaagttcgtcaccgtgaaagactaaccgccgtgctctccaagttggaaaaggttgttcgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccaggacgagcccctggatttgtctgcttcctcacagactgaatatgaggcctctcccccagcaccgccgcagagcgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaaacatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgata

SEQ ID NO: 12 - protein, 20 aa, Porcine reproductive and respiratory syndrome virus
MEQKLISEEDLNRSMNSSGS

SEQ ID NO: 13 - protein, 8 aa, Porcine reproductive and respiratory syndrome virus
LEHHHHHH

SEQ ID NO: 14 - DNA, 545 bp, Porcine reproductive and respiratory syndrome virus
  CDS (175)..(366)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccatgaacgccaacagcaccagcagctcacattttcagttgatttataacttgacgctatgcgagctgaatggcacagattggctggctggaaagtttgattgggcagtgctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 15 - DNA, 108 bp, Porcine reproductive and respiratory syndrome virus
atgaacgccaacagcaccagcagctcacattttcagttgatttataacttgacgctatgcgagctgaatggcacagattggctggctggaaagtttgattgggcagtg

SEQ ID NO: 16 - protein, 64 aa, Porcine reproductive and respiratory syndrome virus
MEQKLISEEDLNRSMNSSGSMNANSTSSSHFQLIYNLTLCELNGTDWLAGKFDWAVLEHHHHHH

SEQ ID NO: 17 - DNA, 545 bp, Porcine reproductive and respiratory syndrome virus
  CDS (175)..(366)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccatgaacgccagcaacgacagcagctcccatctacagctgatttacaacttgacgctatgtgagctgaatggcacagattggctagctaacaaatttgattgggcagtgctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 18 - DNA, 108 bp, Porcine reproductive and respiratory syndrome virus
atgaacgccagcaacgacagcagctcccatctacagctgatttacaacttgacgctatgtgagctgaatggcacagattggctagctaacaaatttgattgggcagtg

SEQ ID NO: 19 - protein, 64 aa, Porcine reproductive and respiratory syndrome virus
MEQKLISEEDLNRSMNSSGSMNASNDSSSHLQLIYNLTLCELNGTDWLANKFDWAVLEHHHHHH

SEQ ID NO: 20 - DNA, 596 bp, Porcine reproductive and respiratory syndrome virus
  CDS (175)..(417)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccatgaacgccagcaacgacagcagctcccatctacagctgatttacaacttgacgctatgtgagctgaatggcacagattggctagctaacaaatttgattgggcagtgctcgagggaggaggcggcagcgggtttgttcacgggcggtatgtcctaagtctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 21 - DNA, 159 bp, Porcine reproductive and respiratory syndrome virus
atgaacgccagcaacgacagcagctcccatctacagctgatttacaacttgacgctatgtgagctgaatggcacagattggctagctaacaaatttgattgggcagtgctcgagggaggaggcggcagcgggtttgttcacgggcggtatgtcctaagt

SEQ ID NO: 22 - protein, 81 aa, Porcine reproductive and respiratory syndrome virus
MEQKLISEEDLNRSMNSSGSMNASNDSSSHLQLIYNLTLCELNGTDWLANKFDWAVLEGGGGSGFVHGRYVLSLEHHHHHH

SEQ ID NO: 23 - protein, 5 aa, Artificial Sequence
  Description of Artificial Sequence Synthetic Gly-Ser linker
GGGGS

SEQ ID NO: 24 - DNA, 656 bp, Porcine reproductive and respiratory syndrome virus
  CDS (175)..(477)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccatgaagaattgcatgtcctggcgctacgcgtgtaccagatataccaactttcttctggacactaagggcagactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtcctctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 25 - DNA, 219 bp, Porcine reproductive and respiratory syndrome virus
atgaagaattgcatgtcctggcgctacgcgtgtaccagatataccaactttcttctggacactaagggcagactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtcct

SEQ ID NO: 26 - protein, 101 aa, Porcine reproductive and respiratory syndrome virus
MEQKLISEEDLNRSMNSSGSMKNCMSWRYACTRYTNFLLDTKGRLYRWRSPVIIEKRGKVEVEGHLIDLKRVVLDGSVATPITRVSAEQWGRPLEHHHHHH

SEQ ID NO: 27 - DNA, 656 bp, Porcine reproductive and respiratory syndrome virus
  CDS (175)..(477)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccatgaagaactgcatgtcctggcgctattcatgtaccagatacaccaacttcctcctagacactaagggcagactctatcgttggcggtcgcctgtcattatagagaaagggggtaaggttgaggtcgaaggccacctgatcgacctcaaaagagttgtgcttgatggttccgtggcaacacctttaaccagagtttcagcggaacaatggggtcgtcctctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 28 - DNA, 219 bp, Porcine reproductive and respiratory syndrome virus
atgaagaactgcatgtcctggcgctattcatgtaccagatacaccaacttcctcctagacactaagggcagactctatcgttggcggtcgcctgtcattatagagaaagggggtaaggttgaggtcgaaggccacctgatcgacctcaaaagagttgtgcttgatggttccgtggcaacacctttaaccagagtttcagcggaacaatggggtcgtcct

SEQ ID NO: 29 - protein, 101 aa, Porcine reproductive and respiratory syndrome virus
MEQKLISEEDLNRSMNSSGSMKNCMSWRYSCTRYTNFLLDTKGRLYRWRSPVIIEKGGKVEVEGHLIDLKRVVLDGSVATPLTRVSAEQWGRPLEHHHHHH

SEQ ID NO: 30 - DNA, 700 bp, Porcine reproductive and respiratory syndrome virus
  CDS (175)..(528)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccatgaacgccagcaacgacagcagctcccatctacagctgatttacaacttgacgctatgtgagctgaatggcacagattggctagctaacaaatttgattgggcagtgctcgagggaggaggcggcagcgggtttgttcacgggcggtatgtcctaagtggaggaggcggcagcatggggtcgtccttagatgacttctgtcatgatagcacggctccacaaaaggtgcttggaggaggcggcagcgcgcactttcagagtacaaataagctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcatt

SEQ ID NO: 31 - DNA, 270 bp, Porcine reproductive and respiratory syndrome virus
atgaacgccagcaacgacagcagctcccatctacagctgatttacaacttgacgctatgtgagctgaatggcacagattggctagctaacaaatttgattgggcagtgctcgagggaggaggcggcagcgggtttgttcacgggcggtatgtcctaagtggaggaggcggcagcatggggtcgtccttagatgacttctgtcatgatagcacggctccacaaaaggtgcttggaggaggcggcagcgcgcactttcagagtacaaataag

SEQ ID NO: 32 - protein, 118 aa, Porcine reproductive and respiratory syndrome virus
MEQKLISEEDLNRSMNSSGSMNASNDSSSHLQLIYNLTLCELNGTDWLANKFDWAVLEGGGGSGFVHGRYVLSGGGGSMGSSLDDFCHDSTAPQKVLGGGGSAHFQSTNKLEHHHHHH

SEQ ID NO: 33 - protein, 6 aa, Artificial Sequence
  Description of Artificial Sequence Synthetic 6xHis tag
HHHHHH

SEQ ID NO: 34 - DNA, 806 bp, Porcine reproductive and respiratory syndrome virus
  CDS (175)..(627)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 35 - DNA, 369 bp, Porcine reproductive and respiratory syndrome virus
atgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagca

SEQ ID NO: 36 - protein, 151 aa, Porcine reproductive and respiratory syndrome virus
MEQKLISEEDLNRSMNSSGSMPNNNGKQQKRKKGDGQPVNQLCQMLGKIIAQQNQSRGKGPGKKNKKKNPEKPHFPLATEDDVRHHFTPSERQLCLSSIQTAFNQGAGTCTLSDSGRISYTVEFSLPTHHTVRLIRVTASPSALEHHHHHH

SEQ ID NO: 37 - DNA, 907 bp, Porcine reproductive and respiratory syndrome virus
  CDS (175)..(735)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccaggacgagcccctggatttgtctgcttcctcacaggctgaatatgaggcctctcccccagcaccgccgcagagcgggggcgttctgggagtagaggggcatgaagctgaggaaaccctgagtgaaatctcggacatgtcgggtaacattaaacctgcgtccgtgtcatcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcatt

SEQ ID NO: 38 - DNA, 477 bp, Porcine reproductive and respiratory syndrome virus
ggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccaggacgagcccctggatttgtctgcttcctcacaggctgaatatgaggcctctcccccagcaccgccgcagagcgggggcgttctgggagtagaggggcatgaagctgaggaaaccctgagtgaaatctcggacatgtcgggtaacattaaacctgcgtccgtgtcatca

SEQ ID NO: 39 - protein, 187 aa, Porcine reproductive and respiratory syndrome virus
MEQKLISEEDLNRSMNSSGSGSPVSLGGDVPNSWEDLAVSSPFDLPTPPEPATPSSELVIVSSPQCIFRPATPLSEPAPIPAPRGTVSRPVTPLSEPIPVPAPRRKFQQVKRLSSAAAIPPYQDEPLDLSASSQAEYEASPPAPPQSGGVLGVEGHEAEETLSEISDMSGNIKPASVSSLEHHHHHH

SEQ ID NO: 40 - DNA, 532 bp, Porcine reproductive and respiratory syndrome virus
  CDS (175)..(360)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccgcgactgctacagtcgctggccgcgctttgtccgttcgtgaaacccggcaggccaaggagcacgaggttgccggcgccaacaaggctgagcacctcaaacacctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcatt

SEQ ID NO: 41 - DNA, 102 bp, Porcine reproductive and respiratory syndrome virus
gcgactgctacagtcgctggccgcgctttgtccgttcgtgaaacccggcaggccaaggagcacgaggttgccggcgccaacaaggctgagcacctcaaacac

SEQ ID NO: 42 - protein, 62 aa, Porcine reproductive and respiratory syndrome virus
MEQKLISEEDLNRSMNSSGSATATVAGRALSVRETRQAKEHEVAGANKAEHLKHLEHHHHHH

SEQ ID NO: 43 - DNA, 538 bp, Porcine reproductive and respiratory syndrome virus
  CDS (175)..(366)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccgcaaagattgacctgtacctccgtggtgcaacaaatcttgaagaatgcttggccaggcttgagaaagcgcgcccgccacgcgtaatcgacacctcctttgattgggatctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcatt

SEQ ID NO: 44 - DNA, 108 bp, Porcine reproductive and respiratory syndrome virus
gcaaagattgacctgtacctccgtggtgcaacaaatcttgaagaatgcttggccaggcttgagaaagcgcgcccgccacgcgtaatcgacacctcctttgattgggat

SEQ ID NO: 45 - protein, 64 aa, Porcine reproductive and respiratory syndrome virus
MEQKLISEEDLNRSMNSSGSAKIDLYLRGATNLEECLARLEKARPPRVIDTSFDWDLEHHHHHH

SEQ ID NO: 46 - DNA, 526 bp, Porcine reproductive and respiratory syndrome virus
  CDS (175)..(354)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccatggggtcgtccttagatgacttctgtcatgatagcacggctccacaaaaggtgcttggaggaggcggcagcgcgcactttcagagtacaaataagctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcatt

SEQ ID NO: 47 - DNA, 96 bp, Porcine reproductive and respiratory syndrome virus
atggggtcgtccttagatgacttctgtcatgatagcacggctccacaaaaggtgcttggaggaggcggcagcgcgcactttcagagtacaaataag

SEQ ID NO: 48 - protein, 60 aa, Porcine reproductive and respiratory syndrome virus
MEQKLISEEDLNRSMNSSGSMGSSLDDFCHDSTAPQKVLGGGGSAHFQSTNKLEHHHHHH

SEQ ID NO: 49 - DNA, 694 bp, Porcine reproductive and respiratory syndrome virus
  CDS (175)..(522)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatcctcagccatagaaacctggaaattcatcacctccagatgccgtttgtgcttgctaggccgcaagtacattctggcccctgcccaccacgttgaaagtgccgcacggtttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcatt

SEQ ID NO: 50 - DNA, 264 bp, Porcine reproductive and respiratory syndrome virus
tcagccatagaaacctggaaattcatcacctccagatgccgtttgtgcttgctaggccgcaagtacattctggcccctgcccaccacgttgaaagtgccgcacggtttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaa

SEQ ID NO: 51 - protein, 116 aa, Porcine reproductive and respiratory syndrome virus
MEQKLISEEDLNRSMNSSGSSAIETWKFITSRCRLCLLGRKYILAPAHHVESAARFHPIAANDNHAFVVRRPGSTTVNGTLVPGLKSLVLGGRKAVKQGVVNLVKYAKLEHHHHHH

SEQ ID NO: 52 - DNA, 821 bp, Porcine reproductive and respiratory syndrome virus
  CDS (175)..(642)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccatggccggtaaaaaccagagccagaagaaaaagaaaagtacagctccgatggggaatggccagccagtcaatcaactgtgccagttgctgggtgcaatgataaagtcccagcgccagcaacctaggggaggacaggccaaaaagaaaaagcctgagaagccacattttcccctggctgctgaagatgacatccggcaccacctcacccagactgaacgctccctctgcttgcaatcgatccagacggctttcaatcaaggcgcaggaactgcgtcgctttcatccagcgggaaggtcagttttcaggttgagtttatgctgccggttgctcatacagtgcgcctgattcgcgtgacttctacatccgccagtcagggtgcaagtctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 53 - DNA, 384 bp, Porcine reproductive and respiratory syndrome virus
atggccggtaaaaaccagagccagaagaaaaagaaaagtacagctccgatggggaatggccagccagtcaatcaactgtgccagttgctgggtgcaatgataaagtcccagcgccagcaacctaggggaggacaggccaaaaagaaaaagcctgagaagccacattttcccctggctgctgaagatgacatccggcaccacctcacccagactgaacgctccctctgcttgcaatcgatccagacggctttcaatcaaggcgcaggaactgcgtcgctttcatccagcgggaaggtcagttttcaggttgagtttatgctgccggttgctcatacagtgcgcctgattcgcgtgacttctacatccgccagtcagggtgcaagt

SEQ ID NO: 54 - protein, 156 aa, Porcine reproductive and respiratory syndrome virus
MEQKLISEEDLNRSMNSSGSMAGKNQSQKKKKSTAPMGNGQPVNQLCQLLGAMIKSQRQQPRGGQAKKKKPEKPHFPLAAEDDIRHHLTQTERSLCLQSIQTAFNQGAGTASLSSSGKVSFQVEFMLPVAHTVRLIRVTSTSASQGASLEHHHHHH

SEQ ID NO: 55 - DNA, 2336 bp, Porcine reproductive and respiratory syndrome virus
  CDS (175)..(234)
  CDS (2134)..(2157)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccgctgccggcaaacgggctcgtgctaagcgtgccgctaaaagtgagaaggattcggctcccacccccaaggttgccctgccggtccccacctgtggaattaccacctactctccaccgacagacgggtcttgtggttggcatgtccttgccgccataatgaaccggatgataaatggtgacttcacgtcccctctgactcagtacaacagaccagaggatgattgggcttctgattatgatcttgttcaggcgattcaatgtctacgactgcctgctaccgtggttcggaatcgcgcctgtcctaacgccaagtaccttataaaacttaacggagttcactgggaggtagaggtgaggtctggaatggctcctcgctccctttctcgtgaatgtgtggttggcgtttgctctgaaggctgtgtcgcaccgccttatccagcagacgggctacctaaacgtgcactcgaggccttggcgtctgcttacagactaccctccgattgtgttagctctggtattgctgactttcttgctaatccacctcctcaggaattctggaccctcgacaaaatgttgacctccccgtcaccagagcggtccggcttctctagtttgtataaattactattagaggttgttccgcaaaaatgcggtgccacggaaggggctttcatctatgctgttgagaggatgttgaaggattgtccgagctccaaacaggccatggcccttctggcaaaaattaaagttccatcctcaaaggccccgtctgtgtccctggacgagtgtttccctacggatgttttagccgacttcgagccagcatctcaggaaaggccccaaagttccggcgctgctgttgtcctgtgttcaccggatgcaaaagagttcgaggaagcagccccggaagaagttcaagagagtggccacaaggccgtccactctgcactccttgccgagggtcctaacaatgagcaggtacaggtggttgccggtgagcaactgaagctcggcggttgtggtttggcagtcgggaatgctcatgaaggtgctctggtctcagctggtctaattaacctggtaggcgggaatttgtccccctcagaccccatgaaagaaaacatgctcaatagccgggaagacgaaccactggatttgtcccaaccagcaccagcttccacaacgacccttgtgagagagcaaacacccgacaacccaggttctgatgccggtgccctccccgtcaccgttcgagaatttgtcccgacggggcctatactctgtcatgttgagcactgcggcacggagtcgggcgacagcagttcgcctttggatctatctgatgcgcaaaccctggaccagcctttaaatctatccctggccgcttggccagtgagggccaccgcgtctgaccctggctgggtccacggtaggcgcgagcctgtctttgtaaagcctcgaaatgctttctctgatggcgattcagcccttcagttcggggagctttctgaatccagctctgtcatcgagtttgaccggacaaaagatgctccggtggttgacgcccctgtcgacttgacgacttcgaacgaggccctctctgtagtcgatcctttcgaatttgccgaactcaagcgcccgcgtttctccgcacaagccttaattgaccgaggcggtccacttgccgatgtccatgcaaaaataaagaaccgggtatatgaacagtgcctccaagcttgtgagcccggtagtcgtgcaaccccagccaccagggagtggctcgacaaaatgtgggatagggtggacatgaaaacttggcgctgcacctcgcagttccaagctggtcgcattcttgcgtccctcaaattcctccctgacatgattcaagacacaccgcctctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 56 - DNA, 1899 bp, Porcine reproductive and respiratory syndrome virus
gctgccggcaaacgggctcgtgctaagcgtgccgctaaaagtgagaaggattcggctcccacccccaaggttgccctgccggtccccacctgtggaattaccacctactctccaccgacagacgggtcttgtggttggcatgtccttgccgccataatgaaccggatgataaatggtgacttcacgtcccctctgactcagtacaacagaccagaggatgattgggcttctgattatgatcttgttcaggcgattcaatgtctacgactgcctgctaccgtggttcggaatcgcgcctgtcctaacgccaagtaccttataaaacttaacggagttcactgggaggtagaggtgaggtctggaatggctcctcgctccctttctcgtgaatgtgtggttggcgtttgctctgaaggctgtgtcgcaccgccttatccagcagacgggctacctaaacgtgcactcgaggccttggcgtctgcttacagactaccctccgattgtgttagctctggtattgctgactttcttgctaatccacctcctcaggaattctggaccctcgacaaaatgttgacctccccgtcaccagagcggtccggcttctctagtttgtataaattactattagaggttgttccgcaaaaatgcggtgccacggaaggggctttcatctatgctgttgagaggatgttgaaggattgtccgagctccaaacaggccatggcccttctggcaaaaattaaagttccatcctcaaaggccccgtctgtgtccctggacgagtgtttccctacggatgttttagccgacttcgagccagcatctcaggaaaggccccaaagttccggcgctgctgttgtcctgtgttcaccggatgcaaaagagttcgaggaagcagccccggaagaagttcaagagagtggccacaaggccgtccactctgcactccttgccgagggtcctaacaatgagcaggtacaggtggttgccggtgagcaactgaagctcggcggttgtggtttggcagtcgggaatgctcatgaaggtgctctggtctcagctggtctaattaacctggtaggcgggaatttgtccccctcagaccccatgaaagaaaacatgctcaatagccgggaagacgaaccactggatttgtcccaaccagcaccagcttccacaacgacccttgtgagagagcaaacacccgacaacccaggttctgatgccggtgccctccccgtcaccgttcgagaatttgtcccgacggggcctatactctgtcatgttgagcactgcggcacggagtcgggcgacagcagttcgcctttggatctatctgatgcgcaaaccctggaccagcctttaaatctatccctggccgcttggccagtgagggccaccgcgtctgaccctggctgggtccacggtaggcgcgagcctgtctttgtaaagcctcgaaatgctttctctgatggcgattcagcccttcagttcggggagctttctgaatccagctctgtcatcgagtttgaccggacaaaagatgctccggtggttgacgcccctgtcgacttgacgacttcgaacgaggccctctctgtagtcgatcctttcgaatttgccgaactcaagcgcccgcgtttctccgcacaagccttaattgaccgaggcggtccacttgccgatgtccatgcaaaaataaagaaccgggtatatgaacagtgcctccaagcttgtgagcccggtagtcgtgcaaccccagccaccagggagtggctcgacaaaatgtgggatagggtggacatgaaaacttggcgctgcacctcgcagttccaagctggtcgcattcttgcgtccctcaaattcctccctgacatgattcaagacacaccgcct

SEQ ID NO: 57 - DNA, 2588 bp, Porcine reproductive and respiratory syndrome virus
  CDS (175)..(234)
  CDS (2386)..(2409)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccgctggaaagagagcaaggaaagcacgctctggtatgaccaccacagtcgctcaccgcgccttgcccgctcgtgaaatccagcaagccaaaaagcacgaggatgccggcgctgataaggctgtgcatctcaggcactattctccgcctgccgacgggaactgtggttggcactgcatttccgccatcgccaaccgaatggtgaattccaaatttgaaactactcttcccgagagggtgagaccttcagatgactgggctactgacgaggaccttgtgaacaccatccaaattctcaagctccctgcggccttggacaggaacggtgcttgtgttggcgccaaatacgtgcttaagctggaaggcgagcattggactgtctctgtgacccttgggatgtccccttctttgctcccccttgaatgtgttcagggctgttgtgagcataagagcggacttggtcccccagatgcggtcgaagttttcggatttgaccctgcctgccttgaccgactggctgaggtaatgcacttgcctagcagtgtcatcccagctgctctggccgaaatgtccggcgaccccaactgtccggcttccccggtcactactgtgtggactgtttcacaattctttgcccgccacagaggaggagagcaccctgatcaggtgcgcttaggaaaaatcatcagcctttgtcaagttgttgaggaatgctgttgccatcagaataaaaccaaccgggccaccccggaagaggttgcggcaaggattgatcagtacctccatggtgcaacaagtcttgaagaatgcttgattaggcttgagagggtttgcccgccgagcgctgcggacaccttctttgattggaatgttgtgctccctggggttggggcttcaactcagacaaccaaacagctccatgtcaaccagtgccgcgctctggttcctgtcgtgactcaagagcctttggacaaagactcagtccctctgaccgccttctcgctgtccaattgctactatcctgcacaaggtgacgaggttcgtcaccgtgagaggctaaactccgtactctctaagctggagggggttgttcgtgaggaatatgggctcacgccaactgaacctggcccgcgacccgcactaccgaacgggctcgtcgaacttaaagaccagatggaggaggatctgctgaaactagtcaacgcccaggcaacttcagaaatgatggcctgggcagccgagcaggttgatctgaaagcttgggtcaaaaactacccacggtggacaccgccaccccctccaccaagagttcagcctcgaaaaacaaagtctgtcaagagcttgccagggaacaaacctgtccccgctccacgcaggaaggtcagatctgattgtggcagcccgattttgatgggcgacaatgttcctgacggtcgggaagatttgactgttggtggcccccttgatctttcgacaccatccgagccgatgacacctctgagtgagcctgcacttatgcccgcgttgcaatatatttctaggccagtgacatctttgagtgtgctggccccagttcctgcaccgcgtagaactgtgtcccgaccggtgacgcccttgagtgagccaatttttgtgtctgcaccgcgacacaaatttcagcaggtggaagaagcgaatctggcggcaacaacgctgacgcaccaggacgaacctctagatttgtctgcatcctcacagactgaatatgaggcttctcccctaacaccactgcagaacatgggtattctggaggtgggggggcaagaagctgaggaagttctgagtgaaatctcggatacactgaatgacatcaaccctgcacctgtgtcatcaagcagctccctgtcaagtgttaagatcacacgcccaaaacactctgctcaagccatcattgactcgggcgggccctgcagtgggcatctccgaagggaaaaagaagcatgcctcagcatcatgcgtgaggcttgtgatgcggctaagcttagtgaccctgccacgcaggaatggctttctcgcatgtgggatagggttgacatgctgacttggcgcaacacgtctgcttaccaggcgttccgcatcttagatggtaggtttgagtttctcccaaagatgatactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 58 - DNA, 2151 bp, Porcine reproductive and respiratory syndrome virus
gctggaaagagagcaaggaaagcacgctctggtatgaccaccacagtcgctcaccgcgccttgcccgctcgtgaaatccagcaagccaaaaagcacgaggatgccggcgctgataaggctgtgcatctcaggcactattctccgcctgccgacgggaactgtggttggcactgcatttccgccatcgccaaccgaatggtgaattccaaatttgaaactactcttcccgagagggtgagaccttcagatgactgggctactgacgaggaccttgtgaacaccatccaaattctcaagctccctgcggccttggacaggaacggtgcttgtgttggcgccaaatacgtgcttaagctggaaggcgagcattggactgtctctgtgacccttgggatgtccccttctttgctcccccttgaatgtgttcagggctgttgtgagcataagagcggacttggtcccccagatgcggtcgaagttttcggatttgaccctgcctgccttgaccgactggctgaggtaatgcacttgcctagcagtgtcatcccagctgctctggccgaaatgtccggcgaccccaactgtccggcttccccggtcactactgtgtggactgtttcacaattctttgcccgccacagaggaggagagcaccctgatcaggtgcgcttaggaaaaatcatcagcctttgtcaagttgttgaggaatgctgttgccatcagaataaaaccaaccgggccaccccggaagaggttgcggcaaggattgatcagtacctccatggtgcaacaagtcttgaagaatgcttgattaggcttgagagggtttgcccgccgagcgctgcggacaccttctttgattggaatgttgtgctccctggggttggggcttcaactcagacaaccaaacagctccatgtcaaccagtgccgcgctctggttcctgtcgtgactcaagagcctttggacaaagactcagtccctctgaccgccttctcgctgtccaattgctactatcctgcacaaggtgacgaggttcgtcaccgtgagaggctaaactccgtactctctaagctggagggggttgttcgtgaggaatatgggctcacgccaactgaacctggcccgcgacccgcactaccgaacgggctcgtcgaacttaaagaccagatggaggaggatctgctgaaactagtcaacgcccaggcaacttcagaaatgatggcctgggcagccgagcaggttgatctgaaagcttgggtcaaaaactacccacggtggacaccgccaccccctccaccaagagttcagcctcgaaaaacaaagtctgtcaagagcttgccagggaacaaacctgtccccgctccacgcaggaaggtcagatctgattgtggcagcccgattttgatgggcgacaatgttcctgacggtcgggaagatttgactgttggtggcccccttgatctttcgacaccatccgagccgatgacacctctgagtgagcctgcacttatgcccgcgttgcaatatatttctaggccagtgacatctttgagtgtgctggccccagttcctgcaccgcgtagaactgtgtcccgaccggtgacgcccttgagtgagccaatttttgtgtctgcaccgcgacacaaatttcagcaggtggaagaagcgaatctggcggcaacaacgctgacgcaccaggacgaacctctagatttgtctgcatcctcacagactgaatatgaggcttctcccctaacaccactgcagaacatgggtattctggaggtgggggggcaagaagctgaggaagttctgagtgaaatctcggatacactgaatgacatcaaccctgcacctgtgtcatcaagcagctccctgtcaagtgttaagatcacacgcccaaaacactctgctcaagccatcattgactcgggcgggccctgcagtgggcatctccgaagggaaaaagaagcatgcctcagcatcatgcgtgaggcttgtgatgcggctaagcttagtgaccctgccacgcaggaatggctttctcgcatgtgggatagggttgacatgctgacttggcgcaacacgtctgcttaccaggcgttccgcatcttagatggtaggtttgagtttctcccaaagatgata

SEQ ID NO: 59 - DNA, 907 bp, Porcine reproductive and respiratory syndrome virus
  CDS (175)..(735)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccggcagcccgattttgatgggcgacaatgttcctgacggtcgggaagatttgcctgttggtggcccccttgatctttcgacaccatccgagccgatgacacctctgagtgagcctgcacctatgcccgcgttgcaatatatttctaggccagtgacacctttgagtgagctggccccagtacctgcaccgcgtagaactgtgtcccgaccggtgacgcccttgagtgagccaatttttgtgtctgcaccgcgacacaaatttcggcaggtggaagaagcgaatctggcggcaacaatgctgacgcaccaggacgaacctctagatttgtctgcatcctcacagactgaatatgaggcttctcccctaacaccactgcagaacatgggtattcttgaggtgggggggcaagaagctgaggaagttctgagtgaaaactcggatacactgaatgacatcaaccctgcacctgtgtcatcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcatt

SEQ ID NO: 60 - DNA, 477 bp, Porcine reproductive and respiratory syndrome virus
ggcagcccgattttgatgggcgacaatgttcctgacggtcgggaagatttgcctgttggtggcccccttgatctttcgacaccatccgagccgatgacacctctgagtgagcctgcacctatgcccgcgttgcaatatatttctaggccagtgacacctttgagtgagctggccccagtacctgcaccgcgtagaactgtgtcccgaccggtgacgcccttgagtgagccaatttttgtgtctgcaccgcgacacaaatttcggcaggtggaagaagcgaatctggcggcaacaatgctgacgcaccaggacgaacctctagatttgtctgcatcctcacagactgaatatgaggcttctcccctaacaccactgcagaacatgggtattcttgaggtgggggggcaagaagctgaggaagttctgagtgaaaactcggatacactgaatgacatcaaccctgcacctgtgtcatca

SEQ ID NO: 61 - protein, 187 aa, Porcine reproductive and respiratory syndrome virus
MEQKLISEEDLNRSMNSSGSGSPILMGDNVPDGREDLPVGGPLDLSTPSEPMTPLSEPAPMPALQYISRPVTPLSELAPVPAPRRTVSRPVTPLSEPIFVSAPRHKFRQVEEANLAATMLTHQDEPLDLSASSQTEYEASPLTPLQNMGILEVGGQEAEEVLSENSDTLNDINPAPVSSLEHHHHHH

SEQ ID NO: 62 - DNA, 644 bp, Porcine reproductive and respiratory syndrome virus
  CDS (175)..(465)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatcctgcatggcctgccgctatgcccgtacccggtttaccaacttcattgtggacgaccgggggagagttcatcgatggaagtctccaatagtggtagaaaaattgggcaaagccgaagtcgatggcaacctcgtcaccatcaaacatgtcgtcctcgaaggggttaaagctcaacccttgacgaggacttcggctgagcaatgggaggccctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcg

SEQ ID NO: 63 - DNA, 207 bp, Porcine reproductive and respiratory syndrome virus
tgcatggcctgccgctatgcccgtacccggtttaccaacttcattgtggacgaccgggggagagttcatcgatggaagtctccaatagtggtagaaaaattgggcaaagccgaagtcgatggcaacctcgtcaccatcaaacatgtcgtcctcgaaggggttaaagctcaacccttgacgaggacttcggctgagcaatgggaggcc

SEQ ID NO: 64 - protein, 97 aa, Porcine reproductive and respiratory syndrome virus
MEQKLISEEDLNRSMNSSGSCMACRYARTRFTNFIVDDRGRVHRWKSPIVVEKLGKAEVDGNLVTIKHVVLEGVKAQPLTRTSAEQWEALEHHHHHH

SEQ ID NO: 65 - DNA, 694 bp, Porcine reproductive and respiratory syndrome virus
  CDS (175)..(522)
aggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggaacaaaaactcatctcagaagaggatctgaatcgatccatgaattctagtggatccagcttcacagagtcatggaagtttatcacttccagatgcagattgtgttgccttggccggcgatacattctggcccctgcccatcacgtagaaagtgctgcaggtctccattcaatctcagcgtctggtaaccgagcatacgctgtgagaaagcccggactaacatcagtgaacggcactctagtaccaggacttcggagcctcgtgctgggcggcaaacgagctgttaaacgaggagtggttaacctcgtcaagtatggccggctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcatt

SEQ ID NO: 66 - DNA, 264 bp, Porcine reproductive and respiratory syndrome virus
agcttcacagagtcatggaagtttatcacttccagatgcagattgtgttgccttggccggcgatacattctggcccctgcccatcacgtagaaagtgctgcaggtctccattcaatctcagcgtctggtaaccgagcatacgctgtgagaaagcccggactaacatcagtgaacggcactctagtaccaggacttcggagcctcgtgctgggcggcaaacgagctgttaaacgaggagtggttaacctcgtcaagtatggccgg

SEQ ID NO: 67 - protein, 116 aa, Porcine reproductive and respiratory syndrome virus
MEQKLISEEDLNRSMNSSGSSFTESWKFITSRCRLCCLGRRYILAPAHHVESAAGLHSISASGNRAYAVRKPGLTSVNGTLVPGLRSLVLGGKRAVKRGVVNLVKYGRLEHHHHHH

SEQ ID NO: 68 - DNA, 20 bp, Artificial Sequence
  Description of Artificial Sequence Synthetic nucleotide sequence
aggtcgtgtactgtcagtca

SEQ ID NO: 69 - DNA, 20 bp, Artificial Sequence
  Description of Artificial Sequence Synthetic nucleotide sequence
acgtggtgaactgccagtga

SEQ ID NO: 70 - protein, 719 aa, Porcine reproductive and respiratory syndrome virus
AGKRARKARSGATATVAGRALSVRETRQAKEHEVAGANKAEHLKHYSPPAEGNCGWHCISAIANRMVNSKFETTLPERVRPPDDWATDEDLVNAIQILRLPAALDRNGACTSAKYVLKLEGEHWTVTVTPGMSPSLLPLECVQGCCEHKGGLGSPDAVEVSGFDPACLDRLAEVMHLPSSAIPAALAEMSGDSNRSASPVTTVWTVSQFFARHSGGNHPDQVRLGKIISLCQVIEDCCCSQNKTNRVTPEEVAAKIDLYLRGATNLEECLARLEKARPPRVIDTSFDWDVVLPGVEAATQTTKLPQVNQCRALVPVVTQKSLDNNSVPLTAFSLANYYYRAQGDEVRHRERLTAVLSKLEGVVREEYGLMPTEPGPRPTLPRGLDELKDQMEEDLLKLANAQATSDMMAWAAEQVDLKTWVKNYPRWTPPPPPPKVQPRKTKPVKSLPERKPVPAPRRKVGSDCGSPVLLGGNVPNSWEDLAVGGPLDLPTPPEPATPLSEPVLVSAPQCIFRPVTPLSEPAPVPAPRGTVSRPVTPLSEPIPVPAPRRKFQQVERANSAAATPTYQDEPLDLSASSQTEYEASPLAPPQNGGVLEVEGQEAEEVLSEISDMLGDIKPASVSSSSSLSSVRITRPKYSAQAIIDSGGPCSGHLQEVKEACLSIMREACDATKLDDPATQEWLSRMWDRVDMLTWRNTSVYQAIRTLDGRLKFLPKMILE

SEQ ID NO: 71 - protein, 719 aa, Porcine reproductive and respiratory syndrome virus
AGKRARKARSCATATVAGRALSVRETRQAKEHEVAGANKAEHLKHYSPPAEGNCGWHCISAIANRMVNSKFETTLPERVRPPDDWATDEDLVNAIQILRLPAALDRNGACTSAKYVLKLEGEHWTVTVTPGMSPSLLPLECVQGCCGHKGGLGSPDAVEVSGFDPACLDRLAEVMHLPSSAIPAALAEMSGDSDRSASPVTTVWTVSQFFARHSGGNHPDQVRLGKIISLCQVIEDCCCSQNKTNRVTPEEVAAKFDLYLRGATNLEECLARLEKARPPRVIDTPFDWDVVLPGVEAATQTIKLPQVNQCRALVPVVTQKSLDNNSVPLTAFSLANYYYRAQGDEVRHRERLTAVLSKLEKVVREEYGLMPTKPGPRPTLPRGLDELKDQMEEDLLKLANAQTTSDMMAWAAEQVDLKTWVKNYPRWTPPPPSPKVQLRKTKPVKSLPKRKPVPAPRRKVGSDCGSPVSLGGDVPNSWEDLAVSSPFDLPTPPEPAIPSSELVIVSSPQCIFRPATPLSEPAPIPAPRGTVSRPVTPLSEPIPVPAPRRKFQQVKRLSSAAAIPPYQNEPLDLSASSQTEYEASPPAPPQSGGVLGVEGHEAEETLSEISDMSGNIKPASVSSSSSLSSVRITRPKYSAQAIIDSGGPCSGHLQEVKETCLSVMREACDATKLDDPATQEWLSRMWDRVDMLTWRNTSVYQVICTLDGMLKFLPKMILE

SEQ ID NO: 72 - protein, 719 aa, Porcine reproductive and respiratory syndrome virus
AGKRARKARSCATATVAGRALSVRETRQAKEHEVAGANKAEHLKHYSPPAEGNCGWHCISAIANRMVNSKFETTLPERVRPPDDWATDEDLVNAIQILRLPAALDRNGACTSAKYVLKLEGEHWTVTVTPGMSPSLLPLECVQGCCGHKGGLGSPDAVEVSGFDPACLDRLAEVMHLPSSAIPAALAEMSGDSDRSASPVTTVWTVSQFFARHSGGNHPDQVRLGKIISLCQVIEDCCCSQNKTNRVTPEEVAAKIDLYLRGATNLEECLARLEKARPPRVIDTFFDWDVVLPGVEAATQTIKLPQVNQCRALVPVVTQKSLDNNSVPLTAFSLANYYYRAQGDEVRHRERLTAVLSKLEKVVREEYGLMPTEPGPRPTLPRGLDELKDQMEEDLLKLANAQTTSDMMAWAVEQVDLKTWVKNYPRWTPPPPPPKVQPRKTKPVKSLPERKPVPAPRRKVGSDCGSPVSLGGDVPNSWEDLAVSSPFDLPTPPEPATPSSELVIVSSPQCIFRPATPLSEPAPIPAPRGTVSRPVTPLSEPIPVPAPRRKFQQVKRLSSAAAIPPYQNEPLDLSASSQTEYEASPPAPPQSGGVLGVEGHEAEETLSEISDMSGNIKPASVSSSSSLSSVRITRPKYSAQAIIDSGGPCSGHLQEVKETCLSVMREACDATKLDDPATQEWLSRMWDRVDMLTWRNTSVYQAICTLNGRLKFLPKMILE

SEQ ID NO: 73 - protein, 719 aa, Porcine reproductive and respiratory syndrome virus
AGKRARKARSCATATVAGRALSVRETRQAKEHEVAGANKAEHLKHYSPPAEGNCGWHCISAIANRMVNSKFETTLPERVRPPDDWATDEDLVNAIQILRLPAALDRNGACTSAKYVLKLEGEHWTVTVTPGMSPSLLPLECVQGCCGHKGGLGSPDAVEVSGFDPACLDRLAEVMHLPSSAIPAALAEMSGDSDRSASPVTTVWTVSQFFARHSGGNHPDQVRLGKIISLCQVIEDCCCSQNKTNRVTPEEVAAKIDLYLRGATNLEECLARLEKARPPRVIDTFFDWDVVLPGVEAATQTIKLPQVNQCRALVPVVTQKSLDNNSVPLTAFSLANHYYRAQGDEVRHRERLTAVLSNLEKVVREEYGLMPTEPGPRPTLPRGLDELKDQMEEDLLKLANAQTTSDMMAWAVEQVDLKTWVKNYPRWTPPPPPPKVQPRKTKPVKSLPERKPVPAPRRKVGSDCGSPVSLGGDVPNSWEDLAVSSPFDLPTPPEPATPSSELVIVSSPQCIFRPATPLSEPAPIPAPRGTVSRPVTPLSEPIPVPAPRRKFQQVKRLSSAAAIPPYQNEPLDLSASSQTEYEASPPAPPQSGGVLGVEGHEAEETLSEISDMSGNIKPASVSSSSSLSSVRITRPKYSAQAIIDSGGPCSGHLQEVKEACLSVMREACDATKLDDPATQEWLSRMWDRVDMLTWRNTSVYQAICTLDGRLKFLPKMILE

SEQ ID NO: 74 - protein, 719 aa, Porcine reproductive and respiratory syndrome virus
AGKRARKARSCATATVAGRALSVRETRQAKEHEVAGANKAEHLKHYSPPAEGNCGWHCISAIANRMVNSKFETTLPERVRPPDDWATDEDLVNAIQILRLPAALDRNGACTSAKYVLKLEGEHWTVTVTPGMSPSLLPLECVQGCCGHKGGLGSPDAVEVSGFDPACLDRLAEVMHLPSSAIPAALAEMSGDSDRSASPVTTVWTVSQFFARHSGGNHPDQVRLGKIISLCQVIEDCCCSQNKTNRVTPEEVAAKIDLYLRGATNLEECLARLEKARPPRVIDTSFDWDVVLPGVEAATQTIKLPQVNQCRALVPVVTQKSLDNNSVPLTAFSLANYYYRAQGDEVRHRERLTAVLSKLEKVVREEYGLMPTEPGPRPTLPRGLDELKDQMEEDLLKLANAQTTSDMMAWAVEQVDLKTWVKNYPRWTPPPPPPKVQPRKTKPVKSLPERKPVPAPRRKVGSDCGSPVSLGGDVPNSWEDLAVSSPFDLPTPPEPATPSSELVIVSSPQCIFRPATPLSEPAPIPAPRGTVSRPVTPLSEPIPVPAPRRKFQQVKRLSSAAAIPPYQDEPLDLSASSQTEYEASPPAPPQSGGVLGVEGHEAEETLSEISDMSGNIKPASVSSSSSLSSVRITRPKYSAQAIIDSGGPCSGHLQEVKETCLSVMREACDATKLDDPATQEWLSRMWDRVDMLTWRNTSVYQAICTLDGRLKFLPKMILE

SEQ ID NO: 75 - protein, 719 aa, Porcine reproductive and respiratory syndrome virus
AGKRARKARSCATATVAGRALSVRETRQAKEHEVAGADKAEHLKHYSPPAEGNCGWHCISAIANRMVNSIFETTLPERVRPPDDWATDDDLANAIQILRLPAALDRNGACTSAKYVLKLEGEHWTVTVTPGMSPSLLPLECVQGCCEHKGGLGSPDAIEVSGFDPACLDWLAEVMHLPSSAIPAALAEMSGDSDRSASPVTTVWTVSQFFARHSGGNHPDQVRLGKIISLCQVIEDCCCSQNKTNRVTPEEVAAKIDLYLRGATNLEECLARLEKARPPRVIDTSFDWDVVLPGVEAATQTNKLPQVNQCRALVPVVTQKSLDNNSVPLTAFSLANYYYRAQGDEVRHRERLTAVLSKLEEVVREEYGLMPTEPGPRPTLPRGLDELKDQMEEDLLRLANAQATSDMMAWAVEQVDLKTWVKNYPRWTPPPPPPKVQPRKTKPVKSLPERKPVPAPRRKVGPDCGSPVSLGGDVPNSWEDLAVSSPLDLPTPPEPATLSSELVIVSSPQCIFRPATPLSEPAPIPAPRGTVSRPVTPLSEPIPVPAPRRKFQQVKRLSSAAAVPLHQNEPLDLSASSQTEYEASPSAPPQSGGVLGVEGHEAEETLSEISDMSGNIKPASVSSSSSLSSVEITRPKYSAQAIIDSGGPCSGHLQGVKETCLSVMREACDATKLDDPATQEWLSRMWDRVDMLTWRNTSVCQAIRTLDGRLKFLPKMILE

SEQ ID NO: 76 - protein, 719 aa, Porcine reproductive and respiratory syndrome virus
AGKRARKARSSATATVAGRALPVRETRQVEEHEVAGANKAEHLKHYSPPAEGNCGWHCISAIGNRMLNSKFETTLPERVRPPDDWATDEDLVNAIQILRLPAALDRNGACASAKYVLKLEGEHWTVTVTPGMSPSLLPLECVQGCCEHKGGLGSPDAVEVPGFDPACLDWLAEVMHLPSNAIPAALAEMSGDSNRPASPVTTVWTVSQFLARHNGGNHPDQIRLGKIISLCQVIEDCCCSQNKTNRVTPEEVAAKIDLYLRGATNLEECLARLEKARPPRVMDTSFDWDVVLPGVEAATQTTELPQVNQCRALVPVVTQKSLDNNSVPLTAFSLANYYYRAQGDEVRHRERLTAVLSKLEGVVREEYGLMPTGPGPRPTLPRGLDELKDQMEVDLLKLANAQMTSDMMAWAVEQVDLKTWVKNYPRWTPPPPPPIVQPRKTKLVKSLPESKPVPAPRRKVRSDCDCPTLSGNNLPDSWEDLAVGCPSDLPTSPEPVTPLSEPASVSAPRRSFRPVKPLSEPVPVPAPRKTVSRPATPLSEPIPVPAPRRKFQQVEKVNPAAATLGCQDEFPDLSASSHTEYEASPLVLPQNGDVLEVEEREAEEILSGISDILDAIKPASASSSSSLSSVAITRPKYSAQAIIDSGGPYSGHLQEVKETCLSIMSEACDVTKLDDPATQEWLSRMWDRVDMLTWRNTSVHQASRTLDDRFKFLPKMILE

SEQ ID NO: 77 - protein, 719 aa, Porcine reproductive and respiratory syndrome virus
AGKRARKARSGMTTTVAHRALPAREIQQAKKHEDAGADKAVHLRHYSPPADGNCGWHCISAIANRMVNSKFETTLPERVRPSDDWATDEDLVNTIQILKLPAALDRNGACVGAKYVLKLEGEHWTVSVTLGMSPSLLPLECVQGCCEHKSGLGPPDAVEVFGFDPACLDRLAEVMHLPSSVIPAALAEMSGDPNCPASPVTTVWTVSQFFARHRGGEHPDQVRLGKIISLCQVVEECCCHQNKTNRATPEEVAARIDQYLHGATSLEECLIRLERVCPPSAADTFFDWNVVLPGVGASTQTTKQLHVNQCRALVPVVTQEPLDKDSVPLTAFSLSNCYYPAQGDEVRHRERLNSVLSKLEGVVREEYGLTPTEPGPRPALPNGLVELKDQMEEDLLKLVNAQATSEMMAWAAEQVDLKAWVKNYPRWTPPPPPPRVQPRKTKSVKSLPGNKPVPAPRRKVRSDCGSPILMGDNVPDGREDLTVGGPLDLSTPSEPMTPLSEPALMPALQYISRPVTSLSVLAPVPAPRFTVSRPVTPLSEPIFVSAPRHKFQQVEEANLAATTLTHQDEPLDLSASSQTEYEASPLTPLQNMGILEVGGQEAEEVLSEISDTLNDINPAPVSSSSSLSSVKITRPKHSAQAIIDSGGPCSGHLRREKEACLSIMREACDAAKLSDPATQEWLSRMWDRVDMLTWRNTSAYQAFRILDGRFEFLPKMILE

SEQ ID NO: 78 - protein, 719 aa, Porcine reproductive and respiratory syndrome virus
AGKRARKARSGMTTTVAHRALPAREIQQAKKHEDAGADKAVHLRHYSPPADGNCGWHCISAIANRMVNSKFETTLPERVRPSDDWATDEDLVNTIQILKLPAALDRNGACVGAKYVLKLEGEHWTVSVTLGMSPSLLPLECVQGCCEHKSGLGPPDAVEVFGFDPACLDRLAEVMHLPSSVIPAALAEMSGDPNCPASPVTTVWTVSQFFARHRGGEHPDQVRLGKIISLCQVVEECCCHQNKTNRATPEEVAARIDQYLHGATSLEECLIRLERVCPPSAADTFFDWNVVLPGVGASTQTTKQLHVNQCRALVPVVTQEPLDKDSVPLTAFSLSNCYYPAQGDEVRHRERLNSVLSKLEGVVREEYGLTPTEPGPRPALPNGLVELKDQMEEDLLKLVNAQATSEMMAWAAEQVDLKAWVKNYPRWTPPPPPPRVQPRKTKSVKSLPGNKPVPAPRRKVRSDCGSPILMGDNVPDGREDLTVGGPLDLSTPSEPMTPLSEPALMPALQYISRPVTSLSVLAPVPAPRRTVSRPVTPLSEPIFVSAPRHKFQQVEEANLAATTLTHQDEPLDLSASSQTEYEASPLTPLQNMGILEVGGQEAEEVLSEISDTLNDINPAPVSSSSSLSSVKITRPKHSAQAIIDSGGPCSGHLRREKEACLSIMRKACDAAKLSDPATQEWLSRMWDRVDMLTWRNTSAYQAFRILDGRFEFLPKMILE

SEQ ID NO: 79 - protein, 719 aa, Porcine reproductive and respiratory syndrome virus
AGKRAKKARSGATATVAHRASPVRETQQAKKHEVANANRAGHFKRYSPPADGNCGWHCISAIANRMVNSKFETTLPERVRPSDDWATDEDLVNTIQILRLPAALDRNGACASAKYVLKLEGEHWTVSVTPGTSPSLLPLECVQGCCEHKGGLGSPDAVEISGFDPACLDRLAEIMHLPSSVIPAALAEMSGDPNRPASPVTTVWTVSQFFARHRGGEHPDQVRLGKIISLCQVIEECCCRQNDTNRVTPEEVAVKINQYLRGATNLEECLTRLERACPPSAADTSFDWNVVLPGIEAATQTTKQLHVNQCRALVPVVTQEPLDKDSVPLTAFSLSNCYYPAQGDEVRHRERLNSVLSKLEGVVREEYGLTPTEPGPRPALPNGLDELKDQMEEDLLKLVNAQATSEMMAWAAEQVDLKAWVKNYPRWTPPPPPPRVQPRKTKSVKSLPGDKPVPAPRRKVRSDCGSPILMGDNDPNGREDLTVDGPLDLSTPSEPMTPLGEPALLPALQHISRPVTSLSVPAPVPAPRRAVSRPVTPLSEPIFESAPRHKLQQVEEANLVATTLTHQDEPLDLSASSQTEYEASPLAPLQNMGVLEVGGQEAEEVLSEISDILNDINPAPVSSSSSLSSVKITRPKYSAQAIIDSGGPCSGHLRREKEACLSIMRKACDAAKLSDPATQEWLSRMWDRVDMLTWRNTSAYQALRVLDGRFGFLPKMILE

SEQ ID NO: 80 - protein, 725 aa, Porcine reproductive and respiratory syndrome virus
AGKRARRARSGATATVAHCALPAREAQQAKKLEVASANRAEHLKYYSPPADGNCGWHCISAITNRMVNSKFETTLPERVRPSDDWATDEDLVNTIQILRLPAALDRNGACAGAKYVLKLEGEHWTVSVTPGMTPSLLPLECVQGCCEHKSGLGFPDVVEVSGFDPACLDRLAEIMHLPSSVIPAALAEMSDDFNRLASPAATVWTVSQFFARHRGGEHPDQVCLGKIINLCQVIEECCCSRNKANRATPEEVAAKVDQYLRGAASLGECLAKLERARPPSAMDTSFDWNVVLPGVETADQTTKQLHVNQCRALVPVVTQEPLDRDSVPLTAFSLSNCYYPAQGDEVRHRERLNSVLSKLEGVVREEYGLTPTGPGPRPALPNGLDELKDQMEEDLLKLVNAQATSEMMAWAAEQVDLKAWVKNYPRWTPPPPPPRVQPRKTKSVKSLLENKPVPAPRRKVRSDYGSPILMGDNVPNGWEDSTVGGPLDLSAPSEPMTPLSEPVLVSAPQCISRPVTSLSVPAPVPAPRRAVSRPMTPSSEPIFVSALRHKFQQVEKANLAAAAPMYQDEPLDLSASSQTEYGASPLTPPQNVGILEVRGQEAEEVLSEISDILNDTNPAPVSSSSSLSSVRITRPKYSAQAIIDLGGPCSGHLQREKEACLRIMREACDAAKLSDPATQEWLSRMWDRVDMLTWRNTSAYQAFRTLDGRFGFLPKMILETPPPYP

SEQ ID NO: 81 - protein, 719 aa, Porcine reproductive and respiratory syndrome virus
AGKRARKARSGATTMVAHRALSARETRQAKKHEGADANKAEHLEHYSPPAEGNCGWHCISAIANRMVNSNFETTLPERARPLDDWATDEDLVNTIQILRLPAALDRNGACTSAKYVLRLEGEHWTVSVTPGMSPSLLPLECVQGCCEHKGGLGSPDAVEVSGFDPACLDRLAEVMHLPSSAIPAALAEMPVDSNRPASPVTTAWTVSQFYARHRGGNHRDQVCLGKIISLCQVIEDCCCHQNKTNRATPEEVAAKIDQYLRGATSLEECLIKLERVSPPSAADTSFDWNVVLPGVEAANQTTKQLHVNQCRALVPVVTQEPLDKDSVPLTAFSLSNCYYPAQGDEVRHRERLNSVLSKLEGVVLEEYGLMSTGLGPRPVLPSGLDELKDQMEEDLLKLANAQATSEMMAWAAEQVDLKAWVKSYPRWTPPPPPPRVQPRKTKPVKSLPENKPVPAPRRKVGSDCGSPILMGDNVPNGWEDFAVGGPLDFPTPSEPMTPLSEPVLMPASQHIPRPVTPLSGPAPVPAPRRTVSRPMTPLSEPIFVSAPRHKFQQVEEANPAATTLTYQDEPLDLSAFSQTECEASPLAPLQNMGILEAGGQEAEEVLSGISDILNDINPAPVSSSSSLSSVRITRPKYSAQAIIDSGGPCSGHLQREKEACLSIMREACDAAKLSDPATQEWLSRMWDRVDMLTWRNTSAYQALHTLDGRSGFLPKMILE

SEQ ID NO: 82 - protein, 200 aa, Porcine reproductive and respiratory syndrome virus
MLGKCLTAGCCSRLLSLWCIVPFCFAVLANASNSSSSHLQLIYNLTLCELNGTDWLANKFDWAVESFVIFPVLTHIVSYGALTTSHFLDTVGLVTVSTAGFVHGRYVLSSIYAVCALAALICFVIRLAKNCMSWRYSCTRYTNFLLDTKGRLYRWRSPVIIEKGGKVEVEGHLIDLKRVVLDGSVATPLTRVSAEQWGRP

SEQ ID NO: 83 - protein, 200 aa, Porcine reproductive and respiratory syndrome virus
MLGKCLTAGWCSQLLSLGCIVPFCFAVLANASNDSSSHVQLIYNLTLCELNGTDWLANKFDWAVESFVIFPVLTHIVSYGALTTSHFLDTVALVTVSTAGFVHGRYVLSSIYAVCALAALTCFVIRFAKNCMSWRYACTRYTNFLLDTKGRLYRWRSPVIIEKRGKVEVEGHLIDLKRVVLDGSVATPITRVSAEQWGRP

SEQ ID NO: 84 - protein, 200 aa, Porcine reproductive and respiratory syndrome virus
MLEKCLTAGCCSQLLSLWCIVPFCFAVLANASNDSSSHLQLIYNLTLCELNGTDWLANKFDWAVESFVIFPVLTHIVSYGALTTSHFLDTVALVTVSTAGFVHGRYVLSSIYAVCALAALTCFVIRFAKNCMSWRYACTRYTNFLLDTKGGLYRWRSPVIIEKRGKVEVEGHLIDLKRVVLDGSVATPITRVSAEQWGRP

SEQ ID NO: 85 - protein, 200 aa, Porcine reproductive and respiratory syndrome virus
MLEKCLTAGCCSQLLSLWCIVPFCFAVLANASNDSSSHLQLIYNLTLCELNGTDWLANKFDWAVESFVIFPVLTHIVSYGALTTSHFLDTVALVTVSTAGFVHGRYVLSSIYAVCALAALTCFVIRFAKNCMSWRYACTRYTNFLLDTKGRLYRWRSPVIIEKRGKVEVEGHLIDLKRVVLDGSVATPITRVSAEQWGRP

SEQ ID NO: 86 - protein, 200 aa, Porcine reproductive and respiratory syndrome virus
MLEKCLTAGCCSRLLSLWCIVPFCFAVLANASNDSSSHLQLIYNLTLCELNGTDWLANKFDWAVESFVIFPVLTHIVSYGALTTSHFLDTVALVTVSTAGFVHGRYVLSSIYAVCALAALTCFVIRFAKNCMSWRYACTRYTNFLLDTKGRLYRWRSPVIIEKRGKVEVEGHLIDLKRVVLDGSVATPITRVSAEQWGRP

SEQ ID NO: 87 - protein, 200 aa, Porcine reproductive and respiratory syndrome virus
MLEKCLTAGCCSRLLSLWCIVPFCFAVLANASNSSSSHLQLIYNLTLCELNGTDWLANRFDWAVESFVIFPVLTHIVSYGALTTSHFLDTVALVTVSTAGFVHGRYVLSSIYAVCALAALTCFVIRFAKNCMSWRYACTRYTNFLLDTKGRLYRWRSPVIIEKRGKVEVEGHLIDLKRVVLDGSVATPITRVSAEQWGRP

SEQ ID NO: 88 - protein, 200 aa, Porcine reproductive and respiratory syndrome virus
MLGKCLTAGCCSRLLSFWCIVPFCFAVLVNASYSSSSHLQLIYNLTLCELNGTDWLANKFDWAVESFVIFPVLTHIVSYGALTTSHFLDTVGLVTVSTAGFYHGRYVLSSIYAVCALAALICFVIRLAKNCMSWRYSCTRYTNFLLDTKGRLYRWRSPVIIEKGGKVEVESHLIDLKRVVLDGSAATPLTRVSAEQWGRP

SEQ ID NO: 89 - protein, 200 aa, Porcine reproductive and respiratory syndrome virus
MLGRCLTAGCCSRLLSLWCIVPFCFAALVNANSNSSSHLQLIYNLTLCELNGTDWLKDKFDWAVETFVIFPVLTHIVSYGALTTSHFLDTVGLVTVSTAGFYHGRYVLSSIYAVCALAALICFVIRLAKNCMSWRYSCTRYTNFLLDTKGRLYRWRSPVIIEKGGKVEVEGHLIDLKRVVLDGSVATPLTRVSAEQWGRL

SEQ ID NO: 90 - protein, 200 aa, Porcine reproductive and respiratory syndrome virus; identical to SEQ ID NO: 89

SEQ ID NO: 91 - protein, 200 aa, Porcine reproductive and respiratory syndrome virus
MLEKCLTAGCCLRLPSLWCIVPFCFAVLVNANNSSSSHFQSIYNLTLCELNGTEWLSEKFDWAVETFVIFPVLTHIVSYGALTTSHFLDTVGLVTVSTAGFLHRRYVLSSVYAVCALAALICFIIRLAKNCMSWRYSCTRYTNFLLDTKGKLYRWRSPVIIEKGGRVEVEGHLIDLKRVVLDGSAATPLTRVSAEQWGRL

SEQ ID NO: 92 - protein, 200 aa, Porcine reproductive and respiratory syndrome virus
MLGKCLTAGCCSRLLSLWCIVPFCFAVLGSANSSSSSHFQLIYNLTLCELNGTDWLAEKFDWAVETFVIFPVLTHIVSYGALTTSHFLDTVGLVTVSTAGFYHGRYVLSSIYAVCALAALICFVIRLAKNCMSWRYSCTRYTNFLLDTKGRLYRWRSPVIIEKGGKVEVEGHLIDLKRVVLDGSVATPLTRVSAEQWGRL

SEQ ID NO: 93 - protein, 200 aa, Porcine reproductive and respiratory syndrome virus
MLGKCLTTGCCSRLLSLWCIVPFCFAVLVNANSNSSSHFQLIYNLTLCELNGTDWLANKFDWAVETFVIFPVLTHIVSYGALTTSHFLDTVGLVTVSTAGFYHGRYVLSSIYAVCALAALICFVIRLAKNCMSWRYSCTRYTNFLLDTKGRLYRWRSPVIVEKGGKVEVEGHLIDLKRVVLDGSVATPLTRVSAEQWGRL

SEQ ID NO: 94 - protein, 123 aa, Porcine reproductive and respiratory syndrome virus
MPNNNGKQQKKKKGDGQPVNQLCQMLGKIIAQQNQSRGKGPGKKNKKKNPEKPHFPLATEDDVRHHFTPSERQLCLSSIQTAFNQGAGTCTLSDSGRISYTVEFSLPTHHTVRLIRVTASPSA

SEQ ID NO: 95 - protein, 123 aa, Porcine reproductive and respiratory syndrome virus
MPNNNGKQQKRKKGDGQPVNQLCQMLGKIIAQQNQSRGKGPGKKNKKKNPEKPHFPLATEDDVRHHFTPSERQLCLSSIQTAFNQGAGTCTLSDSGRISYTVEFSLPTHHTVRLIRVTASPSA

SEQ ID NO: 96 - protein, 123 aa, Porcine reproductive and respiratory syndrome virus; identical to SEQ ID NO: 95

SEQ ID NO: 97 - protein, 123 aa, Porcine reproductive and respiratory syndrome virus; identical to SEQ ID NO: 95

SEQ ID NO: 98 - protein, 123 aa, Porcine reproductive and respiratory syndrome virus; identical to SEQ ID NO: 95

SEQ ID NO: 99 - protein, 123 aa, Porcine reproductive and respiratory syndrome virus; identical to SEQ ID NO: 95

SEQ ID NO: 100 - protein, 123 aa, Porcine reproductive and respiratory syndrome virus
MPNNNGKQQKKKKGDGQPVNQLCQMLGKIIAQQNQSRGKGPGKKNKKKNPEKPHFPLATEYDVRHHFTPSERQLCLSSIQTAFNQGAGTCTLSDSGRISYTVEFSLPTHHTVRLIRVTASPSA

SEQ ID NO: 101 - protein, 123 aa, Porcine reproductive and respiratory syndrome virus
MPNNNGKQQKKKRGNGQPVNQLCQMLGKIIAQQNQSRGKGPGKKIKNKNPEKPHFPLATEDDVRHHFTPSERQLCLSSIQTAFNQGAGTCTLSDSGRISYTVEFSLPTHHTVRLIRVTAPSSA

SEQ ID NO: 102 - protein, 123 aa, Porcine reproductive and respiratory syndrome virus; identical to SEQ ID NO: 101

SEQ ID NO: 103 - protein, 123 aa, Porcine reproductive and respiratory syndrome virus
MPNNNGKQQKKKKGNGQPVNQLCQMLGKIIAQQNQSRGKGPGKKIKKKNPEKPHFPLATEDDVRHHFTPSERQLCLSSIQTAFNQGAGTCTLSDSGRISYTVEFSLPTHHTVRLIRVTAPPSA

SEQ ID NO: 104 - protein, 123 aa, Porcine reproductive and respiratory syndrome virus
MPNNNGKQQKKKKGNGQPVNQLCQMLGKIIAQQNQSRGKGPGKKSKKKNPEKPHFPLATEDDVRHHFTPGERQLCLSSIQTAFNQGAGTCTLSDSGRISYTVEFSLPTHHTVRLIRVTASPSA

SEQ ID NO: 105 - protein, 123 aa, Porcine reproductive and respiratory syndrome virus
MPNNNGKQRKKKKGNGQPVNQLCQMLGKIIAQQNQSRGKGPGKKNKKKSPEKPHFPLATEDDVRHHFTPSERQLCLSSIQTAFNQGAGTCTLSDSGRISYTVEFSLPTHHTVRLIRVTASPSA

(19) United States
(12) Patent Application Publication
(10) Pub. No.: US 2008/0268426 A1
(43) Pub. Date: Oct. 30, 2008

(54) IDENTIFYING VIRALLY INFECTED AND VACCINATED ORGANISMS

(75) Inventors: Michael P. Murtaugh, Shoreview, MN (US); Craig R. Johnson, Eagan, MN (US)

Correspondence Address: FISH & RICHARDSON P.C., PO BOX 1022, MINNEAPOLIS, MN, 55440-1022, US

(73) Assignee: REGENTS OF THE UNIVERSITY OF MINNESOTA, Saint Paul, MN (US)

(21) Appl. No.: 12/138,166

(22) Filed: Jun. 12, 2008

Related U.S. Application Data
(62) Division of application No. 11/155,380, filed on Jun. 17, 2005, now Pat. No. 7,284,432.
(60) Provisional application No. 60/656,192, filed on Feb. 25, 2005.
(60) Provisional application No. 60/581,325, filed on Jun. 18, 2004.

(51) Int. Cl. C12Q 1/70

(52) U.S. Cl. 435/5

(57) ABSTRACT

This document provides methods and materials related to assessing organisms for the presence or absence of anti-virus antibodies. For example, this document provides methods and materials that can be used to determine whether or not an organism (e.g., a member of a swine species such as a pig) contains anti-PRRS virus antibodies. In other embodiments, this document provides methods and materials that can be used to determine if a particular organism received a vaccine version of a virus, was infected with a naturally-occurring version of the virus, or is naïve with respect to the virus.

### CROSS-REFERENCE TO RELATED APPLICATIONS

This application is a divisional of U.S. patent application Ser. No. 11/155,380, filed Jun. 17, 2005, which claims the benefit of U.S. Provisional Application Ser. No. 60/656,192, filed on Feb. 25, 2005, and U.S. Provisional Application Ser. No. 60/581,325, filed on Jun. 18, 2004. The disclosure of the prior applications are incorporated by reference in their entirety.

### BACKGROUND

1. Technical Field

This document relates to methods and materials involved in identifying virally infected or vaccinated organisms (e.g., vertebrates and mammals). For example, this document relates to methods and material for identifying a mammal (e.g., a pig) having antibodies against a virus such as a porcine reproductive and respiratory syndrome (PRRS) virus.

2. Background Information

Organisms infected with a virus can mount an immune response against that infecting virus. Such an immune response can include the production of antibodies that bind to the virus. The presence of antibodies against a virus can indicate that the organism was exposed to that virus. For example, pigs infected with a PRRS virus can contain pig antibodies that bind PRRS virus.

PRRS is a viral disease of pigs, characterized by reproductive failure in sows (e.g., late-term abortions and stillbirths in sows) and respiratory difficulties in piglets (e.g., interstitial pneumonia in nursery pigs) (Collins et al., J. Vet. Diagn. Invest., 4:117-126 (1992) and Wensvoort et al., Vet Q., 13:121-130 (1991)). It was detected in North America in 1987 (Keffaber, Am. Assoc. Swine Pract. Newsl., 1:1-9 (1989) and Hill, Overview and History of Mystery Swine Disease (Swine Infertility and Respiratory syndrome). In: Proceedings of the Mystery Swine Disease Committee Meeting, October 6, Denver Colo., pp. 29-30. Livestock Conservation Institute, Madison, Wis. (1990)) and in Europe in 1990 (Paton et al., Vet Rec., 128:617 (1991)). The causative agent is a small, enveloped positive-stranded RNA virus that is recovered primarily from alveolar macrophages and blood of infected swine. It is a member of the Arteriviridae, which includes equine arteritis virus (EAV; den Boon et al., J. Virol., 65:2910-2920 (1991)), lactate dehydrogenase elevating virus of mice (LDV; Plagemann and Moennig, Adv. Vir. Res., 41:99-192 (1992)), and simian hemorrhagic fever virus (SHFV; Godeny et al., In Proceedings of the 9th International Congress of Virology, p 22, August 8-13, Glasgow, Scotland (1993) and Plagemann, In Fields Virology, 3^{rd} ed., pp. 1105-1120. Edited by B. N. Fields, D. M. Knipe and P. M. Howley. Philadelphia: Lippincott-Raven (1996)), in the Order Nidovirales (Cavanagh, Arch. Virol., 142:629-633 (1997)). Like other arteriviruses, PRRS virus infects predominantly macrophages and establishes a persistent infection in resident macrophages of numerous tissues (Lawson et al., Virus Res., 51:105-113 (1997) and Christopher-Hennings et al., J. Vet. Diag. Invest., 7:456-464 (1995)).

### SUMMARY

This document involves methods and materials related to assessing organisms to determine whether or not the organisms were exposed to a viral vaccine or viral infection. For example, this document provides methods and materials that can be used to determine whether or not an organism (e.g., a member of a swine species such as a pig) contains anti-PRRS virus antibodies. Determining whether or not, for example, pigs contain anti-PRRS virus antibodies can allow pig farmers to identify pigs that can be infected with PRRS virus. This can allow the farmer to separate pigs suspected to be infected with a PRRS virus from those pigs believed to be uninfected. Also, identifying pigs that do not contain anti-PRRS virus antibodies can allow pig farmers to vaccinate the previously uninfected population of pigs as opposed to an entire herd, which could include many previously infected pigs.

In one embodiment, this document provides methods and materials that can be used to determine if a particular organism received a vaccine version of a virus, was infected with a naturally-occurring version of the virus, or is naïve with respect to the virus. Differentiating between vaccinated organisms and organisms infected with a naturally-occurring version of the virus can allow clinicians, in the case of humans, and farmers, in the case of farm animals, to determine the immunological origin of each organism's immunity to the virus. For example, a farmer receiving a herd of pigs can determine if the pigs of the herd received a PRRS virus vaccine, were infected with a naturally-occurring version of the virus (e.g., a field isolate of PRRS virus), or are naïve with respect to the virus. With this information, the farmer can determine whether the herd need not be vaccinated or whether any uninfected pigs are at risk of being infected from, for example, pigs that were infected with a naturally-occurring version of the virus.

In general, this document features a kit for detecting a swine anti-PRRS virus antibody. The kit includes (a) a polypeptide having an amino acid sequence present in a PRRS virus polypeptide selected from the group consisting of NSP 2 polypeptides and ORF 5 polypeptides, wherein the polypeptide contains an epitope for the swine anti-PRRS virus antibody; and (b) an anti-swine Ig antibody. The polypeptide can be at least eight amino acid residues in length. The polypeptide can contain an amino acid sequence at least 100 amino acids in length that is at least about 80 percent identical to an amino acid sequence encoded by the nucleic acid sequence set forth in SEQ ID NO:5 over the length. The polypeptide can contain an amino acid sequence at least 100 amino acids in length that is at least about 90 percent identical to an amino acid sequence encoded by the nucleic acid sequence set forth in SEQ ID NO:5 over the length. The polypeptide can contain an amino acid sequence at least 20 amino acids in length that is at least about 80 percent identical to a sequence set forth in SEQ ID NO:22 over the length. The polypeptide can contain an amino acid sequence at least 20 amino acids in length that is at least about 90 percent identical to a sequence set forth in SEQ ID NO:22 over the length. The polypeptide can contain the amino acid sequence encoded by the nucleic acid sequence set forth in SEQ ID NO:11. The polypeptide can contain an amino acid sequence of SEQ ID NO:32. The polypeptide can contain an amino acid sequence of SEQ ID NO: 16, 19, 22, 26, 29, 32, 39, 45, 61, or 64. The polypeptide can be a recombinant polypeptide produced in cells not infected with a PRRS virus. The anti-swine Ig antibody can be an anti-swine IgG or IgM antibody. The anti-swine Ig antibody can be a goat anti-swine Ig antibody. The kit can contain a polypeptide having an amino acid sequence present in a PRRS virus NSP 2 polypeptide and a polypeptide having an amino acid sequence present in a PRRS virus ORF 5 polypeptide. The kit can contain a polypeptide having an amino acid sequence present in a PRRS virus ORF 7 polypeptide (e.g., a polypeptide containing an amino acid sequence of SEQ ID NO:36 or 54). The kit can contain a polypeptide having an amino acid sequence present in a PRRS virus ORF 6 polypeptide (e.g., a polypeptide containing an amino acid sequence of SEQ ID NO:32, 48, 51, or 67). The anti-swine Ig antibody can contain an enzyme. The kit can contain a polypeptide having an amino acid sequence present in a PRRS virus NSP 1 polypeptide. The kit can contain a control sample containing swine anti-PRRS virus antibody. The kit can contain a control sample containing swine serum lacking swine anti-PRRS virus antibodies.

In another embodiment, this document features a method for determining whether or not a sample contains a swine anti-PRRS virus antibody. The method includes (a) contacting a polypeptide with the sample under conditions wherein the polypeptide forms a polypeptide:swine anti-PRRS virus antibody complex with an antibody, if present, within the sample, wherein the polypeptide contains an amino acid sequence present in a PRRS virus polypeptide selected from the group consisting of NSP 2 polypeptides and ORF 5 polypeptides, wherein the polypeptide contains an epitope for the swine anti-PRRS virus antibody; and (b) detecting the presence or absence of the complex, wherein the presence of the complex indicates that the sample contains the swine anti-PRRS virus antibody. The sample can be a pig serum sample. The polypeptide can be at least eight amino acid residues in length. The polypeptide can contain an amino acid sequence at least 100 amino acids in length that is at least about 80 percent identical to an amino acid sequence encoded by the nucleic acid sequence set forth in SEQ ID NO:5 over the length. The polypeptide can contain an amino acid sequence at least 20 amino acids in length that is at least about 80 percent identical to a sequence set forth in SEQ ID NO:22 over the length. The polypeptide can contain the amino acid sequence encoded by the nucleic acid sequence set forth in SEQ ID NO:11. The polypeptide can contain an amino acid sequence of SEQ ID NO:32. The polypeptide can contain an amino acid sequence of SEQ ID NO: 16, 19, 22, 26, 29, 32, 39, 45, 61, or 64. The polypeptide can be a recombinant polypeptide produced by cells not infected with a PRRS virus. The step (b) can include contacting the complex with an anti-swine Ig antibody. The anti-swine Ig antibody can contain an enzyme. The step (a) can include contacting the sample with polypeptides within a kit, wherein the kit contains a polypeptide having an amino acid sequence present in a PRRS virus NSP 2 polypeptide and a polypeptide having an amino acid sequence present in a PRRS virus ORF 5 polypeptide. The kit can contain a polypeptide containing an amino acid sequence present in a PRRS virus ORF 7 polypeptide (e.g., a polypeptide containing an amino acid sequence of SEQ ID NO:36 or 54), a polypeptide containing an amino acid sequence present in a PRRS virus ORF 6 polypeptide (e.g., a polypeptide containing an amino acid sequence of SEQ ID NO:32, 48, 51, or 67), and a polypeptide containing an amino acid sequence present in a PRRS virus NSP 1 polypeptide. The method can include contacting the sample with an additional polypeptide to form a polypeptide:swine anti-PRRS virus antibody complex, wherein the additional polypeptide contains an amino acid sequence present in a PRRS virus ORF 7 polypeptide, a PRRS virus ORF 6 polypeptide, or a PRRS virus NSP 1 polypeptide.

In another aspect, this document features a kit for determining whether an animal received a vaccine version of a virus or was infected with a naturally-occurring version of the virus. The kit includes (a) a first polypeptide having an amino acid sequence such that antibodies made against the vaccine version of the virus bind the first polypeptide and antibodies made against the naturally-occurring version of the virus bind the first polypeptide, and (b) a second polypeptide having an amino acid sequence such that antibodies made against the vaccine version of the virus bind the second polypeptide and antibodies made against the naturally-occurring version of the virus do not bind the second polypeptide. The animal can be a vertebrate (e.g., an avian or mammalian species). The animal can be a pig or a human. The virus can be a PRRS virus. The vaccine version can be an attenuated PRRS virus. The vaccine version can be the RespPRRS vaccine. The first polypeptide can contain an amino acid sequence present in a C-terminal portion of an ORF 5 polypeptide of a VR2332 or RespPRRS PRRS virus. The second polypeptide can contain an amino acid sequence present in the N-terminal half of an ORF 5 polypeptide of a VR2332 or RespPRRS PRRS virus.

In another embodiment, this document features a method for determining the immunological state of an animal with respect to a virus, wherein the immunological state is that (1) the animal received a vaccine version of the virus, (2) the animal was infected with a naturally-occurring version of the virus, or (3) the animal is immunologically naive with respect to the virus. The method includes (a) contacting a first sample from the animal with a first polypeptide under conditions wherein the first polypeptide forms a first polypeptide:antibody complex with an antibody, if present, within the first sample, wherein the first polypeptide contains an amino acid sequence such that antibodies made against the vaccine version of the virus bind the first polypeptide and antibodies made against the naturally-occurring version of the virus bind the first polypeptide; (b) contacting a second sample from the animal with a second polypeptide under conditions wherein the second polypeptide forms a second polypeptide:antibody complex with an antibody, if present, within the second sample, wherein the second polypeptide contains an amino acid sequence such that antibodies made against the vaccine version of the virus bind the second polypeptide and antibodies made against the naturally-occurring version of the virus do not bind the second polypeptide; and (c) detecting the presence or absence of the first polypeptide:antibody complex and the presence or absence of the second polypeptide:antibody complex, wherein the presence of the first polypeptide:antibody complex and the presence of the second polypeptide:antibody complex indicates that the animal received the vaccine version of the virus, wherein the presence of the first polypeptide:antibody complex and the absence of the second polypeptide:antibody complex indicates that the animal was infected with the naturally-occurring version of the virus, and wherein the absence of the first polypeptide:antibody complex and the absence of the second polypeptide:antibody complex indicates that the animal is immunologically naive with respect to the virus. The animal can be a vertebrate (e.g., an avian or mammalian species). The animal can be a pig or a human. The virus can be a PRRS virus. The vaccine version can be an attenuated PRRS virus. The vaccine version can be the RespPRRS vaccine. The first polypeptide can contain an amino acid sequence present in a C-terminal portion of an ORF 5 polypeptide of a VR2332 or RespPRRS PRRS virus. The second polypeptide can contain an amino acid sequence present in the N-terminal half of an ORF 5 polypeptide of a VR2332 or RespPRRS PRRS virus.

Another aspect of this document features a substantially pure polypeptide having the amino acid sequence of a PRRS virus NSP 2 polypeptide or a fragment of the PRRS virus NSP 2 polypeptide, wherein the fragment is greater than 20 amino acid residues in length.

Another aspect of this document features a substantially pure polypeptide having the amino acid sequence of a PRRS virus NSP 4 polypeptide or a fragment of the PRRS virus NSP 4 polypeptide, wherein the fragment is greater than 20 amino acid residues in length.

Another aspect of this document features a host cell that expresses a PRRS virus NSP 1, NSP 2, or NSP 4 polypeptide. The cell can be a prokaryotic cell (e.g., a bacterial cell).

Another aspect of this document features a method of reducing background signals in an assay capable of detecting PRRS virus antibodies in a swine sample. The assay includes contacting a solid support containing PRRS virus polypeptides with the swine sample. The method includes treating the solid support with a blocking solution at a pH value greater than 8.0 (e.g., greater than 8.5, 9.0, 9.5, 10.0, or 10.5). The blocking solution can be milk (e.g., nonfat dry milk in PBS), protein solutions, or animal serum.

Another aspect of this document features a solid support containing PRRS virus polypeptides. The solid support was treated with a blocking solution at a pH value greater than 8.0 (e.g., greater than 8.5, 9.0, 9.5, 10.0, or 10.5). The blocking solution can be milk (e.g., nonfat dry milk in PBS), protein solutions, or animal serum. The solid support can be a plastic plate (e.g., a 96 well plate), a glass slide, glass or plastic beads, or the like.

Another aspect of this document features a method of increasing the ability of a polypeptide attached to a solid support to react with an antibody that binds the polypeptide. The method includes contacting the solid support with the polypeptide and a lysozyme. The polypeptide can be a PRRS virus polypeptide. The polypeptide can be a PRRS virus ORF 7 polypeptide. The polypeptide can be a recombinant polypeptide produced by cells not infected with a PRRS virus. The antibody can be an anti-PRRS virus polypeptide antibody. The lysozyme can be a chicken egg lysozyme. The polypeptide and the lysozyme can be contacted with the solid support at a ratio of at least 4 ng of the polypeptide per 1 ng of the lysozyme. The lysozyme and the polypeptide can be contacted with the solid support at a ratio of at least 1 ng of the lysozyme per 1 ng of the polypeptide.

Another aspect of this document features a solid support that was treated with a PRRS virus polypeptide and a lysozyme. The polypeptide can be a PRRS virus ORF 7 polypeptide. The polypeptide can be a recombinant polypeptide produced by cells not infected with a PRRS virus. The lysozyme can be a chicken egg lysozyme. The polypeptide and the lysozyme can be contacted with the solid support at a ratio of at least 4 ng of the polypeptide per 1 ng of the lysozyme. The lysozyme and the polypeptide can be contacted with the solid support at a ratio of at least 1 ng of the lysozyme per 1 ng of the polypeptide.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a diagram of a PRRS virus genome. Genomic regions shaded in gray were PCR amplified from VR2332 viral RNA, cloned, and expressed in E. coli BL21 (DE3RP) cells.

FIG. 2 is a listing of a nucleic acid sequence (SEQ ID NO:1) of a pET 24b myc-polypeptide-His construct with the polypeptide being an NSP 1 polypeptide from the VR-2332 strain of PRRS virus. The underlined nucleic acid sequence (SEQ ID NO:2) encodes an NSP 1 polypeptide from the VR-2332 strain of PRRS virus. The shown amino acid sequence (SEQ ID NO:3) is the amino acid sequence from the start site to the start of the NSP 1 polypeptide-encoding region.

FIG. 3 is a listing of a nucleic acid sequence (SEQ ID NO:4) of a pET 24b myc-polypeptide-His construct with the polypeptide being an NSP 2 polypeptide from the VR-2332 strain of PRRS virus. The underlined nucleic acid sequence (SEQ ID NO:5) encodes an NSP 2 polypeptide. The shown amino acid sequence (SEQ ID NO:6) is the amino acid sequence from the start site into the myc tag-encoding region. The LEHHHHHH sequence (SEQ ID NO:13) includes a his tag.

FIG. 4 is a listing of a nucleic acid sequence (SEQ ID NO:7) of a pET 24b myc-polypeptide-His construct with the polypeptide being an NSP 4 polypeptide from the VR-2332 strain of PRRS virus. The underlined nucleic acid sequence (SEQ ID NO:8) encodes an NSP 4 polypeptide. The shown amino acid sequence (SEQ ID NO:9) is an amino acid sequence for a myc-NSP 4-His polypeptide.

FIG. 5 is a graph plotting the fluorescence level of refolded NSP 1 polypeptides detected using an Agilent bioanalyzer. Purified and refolded NSP 1 polypeptide was applied to an Agilent 2100 Bioanalyzer (Agilent Technologies, Palo Alto, Calif.) and analyzed according to the standard protocol on the Protein 50 Assay LabChip kit. The NSP 1 polypeptide resulted in peaks corresponded to 46 kD (intact polypeptide) and 24 and 22 kD PCP1α and PCP 1β, respectively.

FIG. 6 is a graph plotting the fluorescence level of refolded NSP 4 polypeptides detected using an Agilent bioanalyzer. Purified and refolded NSP 4 polypeptide was applied to an Agilent 2100 Bioanalyzer (Agilent Technologies, Palo Alto, Calif.) and analyzed according to the standard protocol on the Protein 50 Assay LabChip kit. The NSP 4 polypeptide resulted in a single peak at 26 kD.

FIG. 7 is a graph plotting the average titer values for antibodies reactive against NSP1 polypeptides (not refolded), NSP 1 polypeptides (refolded), and nucleocapsid (ORF 7) polypeptides (refolded). The time course of anti-NSP 1 or anti-N antibody response was performed using a cohort of 14 pigs that were infected with PRRS virus strain MN30100 and bled at the indicated times.

FIG. 8 is a graph plotting the average titer values for antibodies reactive against NSP 4 polypeptides (not refolded), NSP 4 polypeptides (folded), and nucleocapsid (ORF 7) polypeptides (refolded). The time course of anti-NSP 4 (not refolded) and anti-ORF 7 antibody responses were performed using a cohort of 14 pigs that were infected with PRRS virus strain MN30100, while the anti-NSP 4 (folded) responses were performed in pigs immunized with Ingelac MLV vaccine.

FIG. 9 is a graph plotting the average titer values for antibodies reactive against refolded NSP 1 and NSP 4 polypeptides in pigs immunized with Ingelvac MLV.

FIG. 10 is a graph plotting the sample/positive ratio (S/P ratio) for samples analyzed using a commercially available ELISA kit (IDEXX 2XR kit). The horizontal line intersecting the Y-axis at 0.4 shows the cutoff value for a positive result.

FIG. 11 is a graph plotting the S/P ratios for samples analyzed using a 3′ polypeptide fragment of PRRS virus ORF 5 in an ELISA. The horizontal line intersecting the Y-axis at 0.21 shows the cutoff value for a positive result.

FIG. 12 is a graph plotting the S/P ratios for samples analyzed using a GP5-M chimeric polypeptide in an ELISA. The horizontal line intersecting the Y-axis at 0.5 shows the cutoff value for a positive result.

FIG. 13 contains two bar graphs plotting the absorbance for samples obtained from animals exposed to MLV or MN30100 PRRS viruses. The absorbance values were detected using an ELISA with the indicated polypeptide.

FIG. 14 contains a sequence alignment of PRRS virus NSP 2 polypeptides (SEQ ID NOS: 70-81, respectively, in order of appearance). The nucleic acid encoding the NSP 2 polypeptide of VR-2332 PRRS virus was truncated using a naturally-occurring XhoI restriction site at nucleotides 3490-3495 to generate nucleic acid encoding a truncated NSP 2 polypeptide referred to as an NSP 2P polypeptide.

FIG. 15 contains a sequence alignment of PRRS virus ORF 5 polypeptides (SEQ ID NOS: 82-93, respectively, in order of appearance).

FIG. 16 contains a sequence alignment of PRRS virus ORF 7 polypeptides (SEQ ID NOS: 94-105, respectively, in order of appearance).

FIG. 17 contains photographs of gels of the indicated purified PRRS virus polypeptides.

FIG. 18 contains graphs plotting the absorbance for ELISAs containing the indicated polypeptide. The groups are as set forth in Table 6.

FIG. 19 is a listing of a nucleic acid sequence (SEQ ID NO:10) of a pET 24b myc-polypeptide-His construct with the polypeptide being an NSP 2P polypeptide from the VR-2332 strain of PRRS virus. The underlined nucleic acid sequence (SEQ ID NO:11) encodes an NSP 2P polypeptide. The first amino acid sequence (SEQ ID NO:12) is an amino acid sequence of the myc tag region of a myc-NSP 2P-His polypeptide, while the second amino acid sequence (SEQ ID NO:13) is an amino acid sequence of the His tag region of a myc-NSP 2P-His polypeptide.

FIG. 20 is a listing of a nucleic acid sequence (SEQ ID NO:14) of a pET 24b myc-polypeptide-His construct with the polypeptide being an ORF 5 5′ polypeptide from the MN30100 strain of PRRS virus. The underlined nucleic acid sequence (SEQ ID NO:15) encodes an ORF 5 5′ polypeptide. The amino acid sequence (SEQ ID NO:16) is an amino acid sequence for a myc-ORF 5 5′-His polypeptide.

FIG. 21 is a listing of a nucleic acid sequence (SEQ ID NO:17) of a pET 24b myc-polypeptide-His construct with the polypeptide being an ORF 5 5′ polypeptide from the VR-2332 strain of PRRS virus. The underlined nucleic acid sequence (SEQ ID NO:18) encodes an ORF 5 5′ polypeptide. The amino acid sequence (SEQ ID NO:19) is an amino acid sequence for a myc-ORF 5 5′-His polypeptide.

FIG. 22 is a listing of a nucleic acid sequence (SEQ ID NO:20) of a pET 24b myc-polypeptide-His construct with the polypeptide being an ORF 5 5′ total polypeptide from the VR-2332 strain of PRRS virus. The underlined nucleic acid sequence (SEQ ID NO:21) encodes an ORF 5 total polypeptide. The amino acid sequence (SEQ ID NO:22) is an amino acid sequence for a myc-ORF 5 total-His polypeptide. A linker amino acid sequence (GGGGS; SEQ ID NO:23) is located between the first and second ectodomains of the 5′ region of the ORF 5 polypeptide.

FIG. 23 is a listing of a nucleic acid sequence (SEQ ID NO:24) of a pET 24b myc-polypeptide-His construct with the polypeptide being an ORF 5 3′ polypeptide from the VR-2332 strain of PRRS virus. The underlined nucleic acid sequence (SEQ ID NO:25) encodes an ORF 5 3′ polypeptide. The amino acid sequence (SEQ ID NO:26) is an amino acid sequence for a myc-ORF 5 3′-His polypeptide.

FIG. 24 is a listing of a nucleic acid sequence (SEQ ID NO:27) of a pET 24b myc-polypeptide-His construct with the polypeptide being an ORF 5 3′ polypeptide from the MN30100 strain of PRRS virus. The underlined nucleic acid sequence (SEQ ID NO:28) encodes an ORF 5 3′ polypeptide. The amino acid sequence (SEQ ID NO:29) is an amino acid sequence for a myc-ORF 5 3′-His polypeptide.

FIG. 25 is a listing of a nucleic acid sequence (SEQ ID NO:30) of a pET 24b myc-polypeptide-His construct with the polypeptide being an ORF 5+6 polypeptide from the VR-2332 strain of PRRS virus. The underlined nucleic acid sequence (SEQ ID NO:31) encodes an ORF 5+6 polypeptide. The amino acid sequence (SEQ ID NO:32) is an amino acid sequence for a myc-ORF 5+6-His polypeptide. A first linker amino acid sequence (GGGGS; SEQ ID NO:23) is located between the first and second ectodomains of the 5′ region of the ORF 5 polypeptide. A second linker amino acid sequence is located between the second ectodomain of the 5′ region of the ORF 5 polypeptide and the first ectodomain of the 5′ region of the ORF 6 polypeptide. A third linker amino acid sequence is located between the first and second ectodomains of the 5′ region of the ORF 6 polypeptide.

FIG. 26 is a listing of a nucleic acid sequence (SEQ ID NO:34) of a pET 24b myc-polypeptide-His construct with the polypeptide being an ORF 7 polypeptide from the VR-2332 strain of PRRS virus. The underlined nucleic acid sequence (SEQ ID NO:35) encodes an ORF 7 polypeptide. The amino acid sequence (SEQ ID NO:36) is an amino acid sequence for a myc-ORF 7-His polypeptide.

FIG. 27 is a listing of a nucleic acid sequence (SEQ ID NO:37) of a pET 24b myc-polypeptide-His construct with the polypeptide being an NSP 2HP polypeptide from the VR-2332 strain of PRRS virus. The underlined nucleic acid sequence (SEQ ID NO:38) encodes an NSP 2HP polypeptide. The amino acid sequence (SEQ ID NO:39) is an amino acid sequence for a myc-NSP 2HP-His polypeptide.

FIG. 28 is a listing of a nucleic acid sequence (SEQ ID NO:40) of a pET 24b myc-polypeptide-His construct with the polypeptide being an NSP 2 S1 HP polypeptide from the VR-2332 strain of PRRS virus. The underlined nucleic acid sequence (SEQ ID NO:41) encodes an NSP 2 S1 HP polypeptide. The amino acid sequence (SEQ ID NO:42) is an amino acid sequence for a myc-NSP 2 S1 HP-His polypeptide.

FIG. 29 is a listing of a nucleic acid sequence (SEQ ID NO:43) of a pET 24b myc-polypeptide-His construct with the polypeptide being an NSP 2 S2 HP polypeptide from the VR-2332 strain of PRRS virus. The underlined nucleic acid sequence (SEQ ID NO:44) encodes an NSP 2 S2 HP polypeptide. The amino acid sequence (SEQ ID NO:45) is an amino acid sequence for a myc-NSP 2 S2 HP-His polypeptide.

FIG. 30 is a listing of a nucleic acid sequence (SEQ ID NO:46) of a pET 24b myc-polypeptide-His construct with the polypeptide being an ORF 6 5′ total polypeptide from the VR-2332 strain of PRRS virus. The underlined nucleic acid sequence (SEQ ID NO:47) encodes an ORF 6 5′ total polypeptide. The amino acid sequence (SEQ ID NO:48) is an amino acid sequence for a myc-ORF 6 5′ total-His polypeptide. A linker amino acid sequence (GGGGS; SEQ ID NO:23) is located between the first and second ectodomains of the 5′ region of the ORF 5 polypeptide.

FIG. 31 is a listing of a nucleic acid sequence (SEQ ID NO:49) of a pET 24b myc-polypeptide-His construct with the polypeptide being an ORF 6 3′ polypeptide from the VR-2332 strain of PRRS virus. The underlined nucleic acid sequence (SEQ ID NO:50) encodes an ORF 6 3′ polypeptide. The amino acid sequence (SEQ ID NO:51) is an amino acid sequence for a myc-ORF 6 3′-His polypeptide.

FIG. 32 contains three graphs plotting the absorbance for samples obtained from animals exposed to MN 184, SDSU 73, or EuroPRRS as well as two controls. The absorbance values were detected using an ELISA of VR-2332 polypeptides (A), LV polypeptides (B), or a mixture of both (C).

FIG. 33A contains a Kyte-Doolittle hydrophilicity profile of an NSP 2 polypeptide.

FIG. 33B is a diagram of NSP 2 and fragments of NSP 2. The amino acid numbering is according to VR-2332 orf 1 (GenBank® accession number U87392). The cysteine protease catalytic site and the hydrophobic domain are labeled.

FIG. 34 contains two graphs plotting the absorbance for samples obtained from animals treated as indicated. The absorbance values were detected using an ELISA of NSP 2 HP (ATP) polypeptides (A) or NSP 2P (VR-2332) polypeptides (B).

FIG. 35 contains two 3D graphs plotting the absorbance for positive and negative samples diluted as indicated and assessed with wells having the indicated amount of polypeptide. The pH during the blocking step was either 7.4 (A) or 9.6 (B).

FIG. 36 is a listing of a nucleic acid sequence (SEQ ID NO:52) of a pET 24b myc-polypeptide-His construct with the polypeptide being an ORF 7 polypeptide from the Lelystad virus strain of PRRS virus. The underlined nucleic acid sequence (SEQ ID NO:53) encodes an ORF 7 polypeptide. The amino acid sequence (SEQ ID NO:54) is an amino acid sequence for a myc-ORF 7-His polypeptide.

FIG. 37 is a listing of a nucleic acid sequence (SEQ ID NO:55) of a pET 24b myc-polypeptide-His construct with the polypeptide being an NSP 2P polypeptide from the Lelystad virus strain of PRRS virus. The underlined nucleic acid sequence (SEQ ID NO:56) encodes an NSP 2P polypeptide. The shown amino acid sequence is the amino acid sequence from the start site to the start of the NSP 2P polypeptide-encoding region (SEQ ID NO:3) and from the end of the NSP 2P polypeptide-encoding region through the his tag (SEQ ID NO:13).

FIG. 38 is a listing of a nucleic acid sequence (SEQ ID NO:57) of a pET 24b myc-polypeptide-His construct with the polypeptide being an NSP 2P polypeptide from the JA 142 virus strain of PRRS virus. The underlined nucleic acid sequence (SEQ ID NO:58) encodes an NSP 2P polypeptide. The shown amino acid sequence is the amino acid sequence from the start site to the start of the NSP 2P polypeptide-encoding region (SEQ ID NO:3) and from the end of the NSP 2P polypeptide-encoding region through the his tag (SEQ ID NO:13).

FIG. 39 is a listing of a nucleic acid sequence (SEQ ID NO:59) of a pET 24b myc-polypeptide-His construct with the polypeptide being an NSP 2HP polypeptide from the Boehringer Ingelheim Ingelvac ATP virus strain of PRRS virus. The underlined nucleic acid sequence (SEQ ID NO:60) encodes an NSP 2HP polypeptide. The amino acid sequence (SEQ ID NO:61) is an amino acid sequence for a myc-NSP 2HP-His polypeptide.

FIG. 40 is a listing of a nucleic acid sequence (SEQ ID NO:62) of a pET 24b myc-polypeptide-His construct with the polypeptide being an ORF 5 3′ polypeptide from the Lelystad virus strain of PRRS virus. The underlined nucleic acid sequence (SEQ ID NO:63) encodes an ORF 5 3′ polypeptide. The amino acid sequence (SEQ ID NO:64) is an amino acid sequence for a myc-ORF 5 3′-His polypeptide.

FIG. 41 is a listing of a nucleic acid sequence (SEQ ID NO:65) of a pET 24b myc-polypeptide-His construct with the polypeptide being an ORF 6 3′ polypeptide from the Lelystad virus strain of PRRS virus. The underlined nucleic acid sequence (SEQ ID NO:66) encodes an ORF 6 3′ polypeptide. The amino acid sequence (SEQ ID NO:67) is an amino acid sequence for a myc-ORF 6 3′-His polypeptide.

FIG. 42 is a graph plotting the absorbance versus the amount of chicken egg lysozyme added to 100 ng of refolded myc-ORF 7-His polypeptide. Values are the specific anti-ORF 7 polypeptide means after subtraction of negative serum backgrounds. Data points are from sera diluted 1/300 (diamond), 1/600 (square), 1/1200 (triangle), and 1/2400 (cross-x).

FIG. 43 is a graph plotting the change in absorbance detected in animals using the indicated ELISAs. VR indicates the polypeptide is from strain VR2332. LV indicates the polypeptide is from Lelystad virus. Standard deviation of the residuals is a measure of goodness-of-fit of the data to the equation determined by linear regression.

FIG. 44 contains graphs plotting the absorbance observed with samples inoculated with the indicated PRRS virus using ELISAs containing the indicated polypeptide.

### DETAILED DESCRIPTION

This document provides methods and materials related to assessing organisms to determine whether or not the organisms were exposed to viral antigens via, for example, a viral vaccination (e.g., vaccination with a vaccine of recombinant viral polypeptides or a vaccine of attenuated virus) or a viral infection. For example, this document provides polypeptides, nucleic acid encoding such polypeptides, methods for making such polypeptides, host cells that express such polypeptides, methods for making such host cells, kits for detecting anti-PRRS virus antibodies, methods for detecting anti-PRRS virus antibodies, kits for assessing an organism's immunological state with respect to a virus, and methods for assessing an organism's immunological state.

### Polypeptides

In one embodiment, this document provides polypeptides that can be used to detect anti-PRRS virus antibodies present in a sample from an organism (e.g., pigs). The anti-PRRS virus antibodies can be any type of anti-PRRS virus antibody. For example, the anti-PRRS virus antibodies can be IgA, IgD, IgE, IgG, or IgM antibodies. Such antibodies can be formed in an organism when that organism is exposed to a PRRS virus antigen such as a PRRS virus polypeptide, an attenuated PRRS virus vaccine, or a pathogenic PRRS virus. In addition, the anti-PRRS virus antibodies can be antibodies that bind to any type of PRRS virus including, without limitation, a VR-2332 PRRS virus (GenBank® Accession No. PRU87392; U.S. Pat. Nos. 5,846,805 and 5,683,865), an MN30100 PRRS virus (Bierk et al., Vet. Rec., 148:687-690 (2001)), an attenuated PRRS virus such as a RespPRRS virus (GenBank® Accession No. AF066183), a 16244B PRRS virus (GenBank® Accession No. AF046869), a PA8 PRRS virus (GenBank® Accession No. AF176348), an SP PRRS virus (GenBank® Accession No. AF184212), an NVSL 97-7985 IA 1-4-2 PRRS virus (GenBank® Accession No. AF325691), a P129 PRRS virus (GenBank® Accession No. AF494042), a CH-1a PRRS virus (GenBank® Accession No. AY032626), a JA142 PRRS virus (GenBank® Accession No. AY424271), an NVSL 97-7895 PRRS virus (GenBank® Accession No. AY545985), or a PL97-1 PRRS virus (GenBank® Accession No. AY585241). Likewise, the anti-PRRS virus antibodies can be antibodies that bind to field isolates or naturally-occurring versions of a PRRS virus including, without limitation, isolates and naturally-occurring versions of PRRS viruses from North America, Europe, or elsewhere (e.g., China).

The polypeptides provided herein can be used to detect anti-PRRS virus antibodies present in a sample from an organism that is susceptible to a PRRS virus infection. Such organisms include, without limitation, swine species such as domestic and feral pigs and wild boars. In some cases, the polypeptides provided herein can be used to detect anti-PRRS virus antibodies present in a sample from an organism that is not susceptible to a PRRS virus infection. For example, the polypeptides provided herein can be used to detect anti-PRRS virus antibodies present in a sample from a rabbit or mouse that was exposed to a PRRS virus antigen via, for example, injection of a PRRS virus polypeptide, an attenuated PRRS virus vaccine, or a pathogenic PRRS virus. When making anti-PRRS virus antibodies in a rabbit or mouse, detecting anti-PRRS virus antibodies in a rabbit or mouse serum sample can help scientists identify rabbits or mice that produce anti-PRRS virus antibodies.

Any sample can be obtained from an organism and assessed for the presence or absence of an anti-PRRS virus antibody. Such samples include, without limitation, blood samples, serum samples, tissue samples (e.g., lymph tissue, muscle tissue, and skin tissue). For example, blood samples can be obtained from pigs and assessed for the presence or absence of pig anti-PRRS virus antibodies.

The polypeptides provided herein can be any length (e.g., between 8 and 2500 amino acid residues). In some embodiments, the polypeptide can contain at least eight amino acid residues. For example, the length of a polypeptide can be greater than 8, 9, 10, 11, 12, 13, 14, 15, 20, 25, 30, 50, 75, 100, 150, 200, 250, 300, 400, 500, 600, 700, 800, 900, 1000, or more amino acid residues. In other embodiments, the length of the polypeptide can be between 25 and 800 amino acid residues, between 50 and 800 amino acid residues, between 50 and 450 amino acid residues, between 50 and 400 amino acid residues, between 50 and 300 amino acid residues, between 100 and 400 amino acid residues, or between 100 and 300 amino acid residues.

The polypeptides can have any amino acid sequence. For example, a polypeptide can contain an amino acid sequence present in a PRRS virus polypeptide (e.g., an NSP 1, NSP 2, NSP 3, NSP 4, NSP 5, NSP 6, pol, C/H, HEL, CORONA, ORF 2, ORF 2b, ORF 3, ORF 4, ORF 5, ORF 6, or ORF 7 polypeptide). In some embodiments, the polypeptide can be a PRRS virus NSP 2 polypeptide that lacks a hydrophobic region such as the region encoded by nucleotides 1339 to 3495 of the sequence set forth in GenBank accession number PRU87392. In other embodiments, the polypeptide can be an ectodomain of a PRRS virus ORF 5 or ORF 6 polypeptide. For example, a polypeptide can contain the first ectodomain from the 5′ end of a PRRS virus ORF 5 polypeptide (ORF 5 5′ ectodomain 1), the second ectodomain from the 5′ end of a PRRS virus ORF 5 polypeptide (ORF 5 5′ ectodomain 2), the first ectodomain from the 5′ end of a PRRS virus ORF 6 polypeptide (ORF 6 5′ ectodomain 1), the second ectodomain from the 5′ end of a PRRS virus ORF 6 polypeptide (ORF 6 5′ ectodomain 2), or combinations thereof. When a polypeptide contains more than one (e.g., two, three, four, five, six, or more) ectodomain, the ectodomains can be next to each other or separated by a linker sequence (e.g., a GGGGS (SEQ ID NO: 23) amino acid linker sequence).

The polypeptides provided herein can contain additional amino acid sequences including those commonly used as tags (e.g., poly-histidine tags, myc tags, GFP tags, and GST tags). For example, a 50 amino acid fragment of a PRRS virus NSP 2 polypeptide can contain the amino acid sequence of a polyhistidine tag (e.g., HHHHHH, SEQ ID NO:33).

A polypeptide provided herein can contain an amino acid sequence having (1) a length, and (2) a percent identity to an identified amino acid sequence over that length. Likewise, an isolated nucleic acid provided herein can encode such a polypeptide or can contain a nucleic acid sequence having (1) a length, and (2) a percent identity to an identified nucleic acid sequence over that length. Typically, the identified nucleic acid or amino acid sequence is a sequence referenced by a particular sequence identification number or a particular GenBank accession number or is a particular PRRS virus nucleic acid or polypeptide (e.g., a PRRS virus NSP 2 polypeptide). The nucleic acid or amino acid sequence being compared to the identified sequence typically is referred to as the target sequence. For example, an identified sequence can be a PRRS virus ORF 5 polypeptide sequence set forth in SEQ ID NO:16, 19, or 22.

A length and percent identity over that length for any nucleic acid or amino acid sequence is determined as follows. First, a nucleic acid or amino acid sequence is compared to the identified nucleic acid or amino acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained from the State University of New York—Old Westbury campus library (catalog number: QH 447.M6714) as well as from Fish & Richardson's web site (“fr” dot “com/blast/”) or from the U.S. government's National Center for Biotechnology Information web site (“ncbi” dot “nlm” dot “nih” dot “gov”). Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -l; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q -1-r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides or amino acid residues from the target sequence presented in alignment with sequence from the identified sequence starting with any matched position and ending with any other matched position. A matched position is any position where an identical nucleotide or amino acid residue is presented in both the target and identified sequence. Gaps presented in the target sequence are not counted since gaps are not nucleotides or amino acid residues. Likewise, gaps presented in the identified sequence are not counted since target sequence nucleotides or amino acid residues are counted, not nucleotides or amino acid residues from the identified sequence.

The percent identity over a determined length is determined by counting the number of matched positions over that length and dividing that number by the length followed by multiplying the resulting value by 100. For example, if (1) a 1000 nucleotide target sequence is compared to a PRRS virus NSP 2 polypeptide sequence, (2) the Bl2seq program presents 200 nucleotides from the target sequence aligned with a region of the PRRS virus NSP 2 polypeptide sequence where the first and last nucleotides of that 200 nucleotide region are matches, and (3) the number of matches over those 200 aligned nucleotides is 180, then the 1000 nucleotide target sequence contains a length of 200 and a percent identity over that length of 90 (i.e., 180÷200*100=90).

It will be appreciated that a single nucleic acid or amino acid target sequence that aligns with an identified sequence can have many different lengths with each length having its own percent identity. For example, a target sequence containing a 20 nucleotide region that aligns with an identified sequence as follows has many different lengths including those listed in Table A.

TABLE A

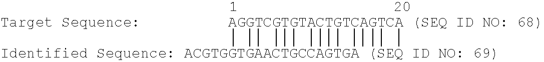

| Starting | Ending | | Matched | Percent |
|---|---|---|---|---|
| Position | Position | Length | Positions | Identity |
| 1 | 20 | 20 | 15 | 75.0 |
| 1 | 18 | 18 | 14 | 77.8 |
| 1 | 15 | 15 | 11 | 73.3 |
| 6 | 20 | 15 | 12 | 80.0 |
| 6 | 17 | 12 | 10 | 83.3 |
| 6 | 15 | 10 | 8 | 80.0 |
| 8 | 20 | 13 | 10 | 76.9 |
| 8 | 16 | 9 | 7 | 77.8 |

It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It is also noted that the length value will always be an integer.

In some embodiments, the polypeptide can have an amino acid sequence at least about 70 percent (e.g., at least about 75, 80, 85, 90, 95, or 99 percent) identical to the sequence set forth in SEQ ID NO:9, 16, 19, 22, 26, 29, 32, 36, 39, 42, 45, 48, 51, 54, 61, 64, or 67 over a length such as 20, 30, 40, 50, 60, 70, 80, 90, 100, 110, 120, 130, 140, 150, 160, 170, 180, 190, 200, or more amino acid residues.

The polypeptides provided herein can be substantially pure. The term “substantially pure” as used herein with reference to a polypeptide means the polypeptide is substantially free of other polypeptides, lipids, carbohydrates, and nucleic acid with which it is naturally associated. For example, a substantially pure polypeptide is any polypeptide that is removed from its natural environment and is at least 60 percent pure. The term “substantially pure” as used herein with reference to a polypeptide also includes chemically synthesized polypeptides. A substantially pure polypeptide can be at least about 65, 70, 75, 80, 85, 90, 95, or 99 percent pure. Typically, a substantially pure polypeptide will yield a single major band on a non-reducing polyacrylamide gel.

Any method can be used to obtain a polypeptide or a substantially pure polypeptide. For example, common polypeptide purification techniques such as affinity chromatography and HPLC as well as polypeptide synthesis techniques can be used. In addition, any material can be used as a source to obtain a substantially pure polypeptide. For example, cultured cells engineered to over-express a particular polypeptide of interest can be used to obtain substantially pure polypeptide. Such cells can be prokaryotic cells (e.g. bacterial cells such as E. coli cells) or eukaryotic cells (e.g., yeast cells, insect cells, mammalian cells). A polypeptide can be designed to contain an amino acid sequence that allows the polypeptide to be captured onto an affinity matrix. For example, a tag such as c-myc, hemagglutinin, poly histidine, or Flag™ tag (Kodak) can be used to aid polypeptide purification. Such tags can be inserted anywhere within the polypeptide including at either the carboxyl or amino termini. Other fusions that could be useful include enzymes that aid in the detection of the polypeptide, such as alkaline phosphatase.

The polypeptides provided herein can be formulated into a polypeptide composition that contains additional ingredients. For example, a polypeptide provided herein can be combined with other polypeptides to form a composition that contains more than one different polypeptide (e.g., two, three, four, five, six, seven, eight, nine, ten, or more different polypeptides). For example, a composition can contain a PRRS virus NSP 2 polypeptide and a PRRS virus NSP 1 polypeptide. A composition containing one or more of the polypeptides provided herein can contain one or more carriers such as a solvent, suspending agent, or any other vehicle. Carriers can be liquid or solid, and can be selected with the desired use in mind so as to provide for the desired bulk, consistency, and other pertinent transport and chemical properties. Typical carriers include, without limitation, water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

### Nucleic Acids

The term “nucleic acid” as used herein encompasses both RNA and DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism or virus from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

The term “exogenous” as used herein with reference to nucleic acid and a particular cell refers to any nucleic acid that does not originate from that particular cell as found in nature. Thus, all non-naturally-occurring nucleic acid is considered to be exogenous to a cell once introduced into the cell. It is important to note that non-naturally-occurring nucleic acid can contain nucleic acid sequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a cell once introduced into the cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid.

Nucleic acid that is naturally occurring can be exogenous to a particular cell. For example, an entire chromosome isolated from a cell of person X is an exogenous nucleic acid with respect to a cell of person Y once that chromosome is introduced into Y's cell.

An isolated nucleic acid can encode any of the polypeptides provided herein. For example, an isolated nucleic acid can encode a PRRS virus NSP 2 polypeptide that lacks a hydrophobic region (e.g., amino acid residues 1 to 722 of a VR-2332 PRRS virus NSP 2 polypeptide) normally present in a PRRS virus NSP 2 polypeptide. In some embodiments, the nucleic acid can encode a polypeptide having an amino acid sequence at least about 70 percent (e.g., at least about 75, 80, 85, 90, 95, or 99 percent) identical to the sequence set forth in SEQ ID NO: 9, 16, 19, 22, 26, 29, 32, 36, 39, 42, 45, 48, 51, 54, 61, 64, or 67 over a length such as 20, 30, 40, 50, 60, 70, 80, 90, 100, 110, 120, 130, 140, 150, 160, 170, 180, 190, 200, or more amino acid residues. In other embodiments, the nucleic acid can have a nucleic acid sequence at least about 70 percent (e.g., at least about 75, 80, 85, 90, 95, or 99 percent) identical to the sequence set forth in SEQ ID NO:2, 5, 8, 11, 15, 18, 21, 25, 28, 31, 35, 38, 41, 44, 47, 50, 53, 56, 58, 60, 63, or 66 over a length such as 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, or more nucleotides.

The isolated nucleic acids provided herein can be at least about 5 bases in length (e.g., at least about 5, 6, 7, 8, 9, 10, 15, 20, 25, 30, 40, 50, 60, 100, 250, 500, 750, 1000, 1500, 2000, 3000, 4000, or 5000 bases in length) and hybridize, under hybridization conditions, to the sense or antisense strand of a nucleic acid that encodes a polypeptide provided herein (e.g., a PRRS virus NSP 2P polypeptide or a PRRS virus ORF 5/ORF 6 chimeric polypeptide). The hybridization conditions can be moderately or highly stringent hybridization conditions.

For the purpose of this invention, moderately stringent hybridization conditions mean the hybridization is performed at about 42° C. in a hybridization solution containing 25 mM KPO₄ (pH 7.4), 5×SSC, 5×Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 50° C. with a wash solution containing 2×SSC and 0.1% sodium dodecyl sulfate.

Highly stringent hybridization conditions mean the hybridization is performed at about 42° C. in a hybridization solution containing 25 mM KPO₄ (pH 7.4), 5×SSC, 5×Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 65° C. with a wash solution containing 0.2×SSC and 0.1% sodium dodecyl sulfate.

Isolated nucleic acids can be obtained using any method including, without limitation, common molecular cloning and chemical nucleic acid synthesis techniques. For example, PCR can be used to obtain an isolated nucleic acid containing a nucleic acid sequence sharing similarity to a PRRS virus nucleic acid sequence provided, for example, in GenBank® (e.g., GenBank® Accession No. PRU87392). PCR refers to a procedure or technique in which target nucleic acid is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. Using PCR, a nucleic acid sequence can be amplified from RNA or DNA. For example, a nucleic acid sequence can be isolated by PCR amplification from total cellular RNA, total genomic DNA, and cDNA as well as from bacteriophage sequences, plasmid sequences, viral sequences, and the like. When using RNA as a source of template, reverse transcriptase can be used to synthesize complimentary DNA strands.

In addition, nucleic acid and amino acid databases (e.g., GenBank®) can be used to obtain an isolated nucleic acid. For example, any nucleic acid sequence having some homology to a nucleic acid sequence that encodes a polypeptide provided herein can be used as a query to search GenBank®.

### Host Cells

A host cell can be designed to contain an isolated nucleic acid described herein. Such cells can be prokaryotic cells (e.g., bacterial cells such as E. coli, B. subtilis, or Agrobacterium tumifaciens, Streptomyces species cells) or eukaryotic cells (e.g., fungal cells such as yeast cells including, without limitation, Saccharomyces species cells and Pichia pastoris cells; insect cells; or mammalian cells). Cells It is noted that cells containing an isolated nucleic acid provided herein are not required to express a polypeptide. In addition, the isolated nucleic acid can be integrated into the genome of the cell or maintained in an episomal state. Thus, host cells can be stably or transiently transfected with a construct containing an isolated nucleic acid provided herein. Typically, a host cell contains an exogenous nucleic acid molecule that encodes a polypeptide provided herein and expresses that encoded polypeptide.

Any methods can be used to introduce an isolated nucleic acid molecule into a cell. For example, calcium phosphate precipitation, electroporation, heat shock, lipofection, microinjection, and viral-mediated nucleic acid transfer are common methods that can be used to introduce an isolated nucleic acid molecule into a cell.

### Detecting Anti-PRRS Virus Antibodies

The methods and materials provided herein can be used to detect anti-PRRS virus antibodies within an organism (e.g., a pig). In general, anti-PRRS virus antibodies are detected by contacting a PRRS virus polypeptide provide herein with a sample from an organism under conditions wherein the PRRS virus polypeptide can bind to an anti-PRRS virus antibody, if present within the sample, to form an antibody-polypeptide complex. Such complexes can be detected using, for example, labeled-antibodies that bind to that organism's antibodies.

Any of the PRRS virus polypeptides provided herein can be used to detect anti-PRRS virus antibodies. Furthermore, multiple different PRRS virus polypeptides provided herein can be used in combination to detect anti-PRRS virus antibodies. For example, a kit containing PRRS virus NSP 1, NSP 2, NSP 4, and ORF 7 polypeptides can be used to detect anti-PRRS virus antibodies.

Typically, the PRRS virus polypeptides are immobilized on solid substrates such as dipsticks, microtiter plates, particles (e.g., beads), affinity columns, and immunoblot membranes. See, U.S. Pat. Nos. 5,143,825; 5,374,530; 4,908,305; and 5,498,551 for exemplary descriptions of solid substrates and methods for their use. For example, PRRS virus polypeptides can be immobilized on a solid substrate, such as a 96-well plate, using known methodologies, then contacted with a sample from a pig under conditions such that anti-PRRS virus antibodies present within the sample can bind to the immobilized PRRS virus polypeptides to form antibody-polypeptide complexes. Suitable conditions include incubation in an appropriate buffer (e.g., sodium phosphate buffer, pH 7.2 to 7.4) at room temperature from about at least 10 minutes to about 10 hours (e.g., from about 1 to about 2.5 hours). Thereafter, unbound material is washed away, and antibody-polypeptide complexes can be detected.

Detecting the presence of such antibody-polypeptide complexes can be indicative of a PRRS virus infection. Any method can be used to detect the antibody-polypeptide complexes. For example, an indicator molecule having binding affinity for the antibody-polypeptide complex can be used to detect an antibody-polypeptide complex. As used herein, an “indicator molecule” is any molecule that allows the presence of a given polypeptide, antibody, or antibody-polypeptide complex to be visualized, either with the naked eye or an appropriate instrument. Typically, the indicator molecule is an antibody having binding affinity for antibodies from the organism (e.g., a pig) from which the sample was obtained, e.g., anti-pig IgG antibodies. Indicator molecules can be detected either directly or indirectly by standard methodologies. See, e.g., Current Protocols in Immunology, Chapters 2 and 8, Coligan et al., (eds.), John Wiley & Sons (1996). For direct detection, the indicator molecule can be labeled with a radioisotope, fluorochrome, other non-radioactive label, or any other suitable chromophore. For indirect detection methods, enzymes such as horseradish peroxidase (HRP) and alkaline phosphatase (AP) can be attached to the indicator molecule, and the presence of the antibody-polypeptide complex can be detected using standard assays for HRP or AP. Alternatively, the indicator molecule can be attached to avidin or streptavidin, and the presence of the antibody-polypeptide complex can be detected with biotin conjugated to, for example, a fluorochrome, or vice versa. Thus, assay formats for detecting antibody-polypeptide complexes can include enzyme-linked immunoassays (ELISA) such as competitive ELISAs, radioimmunoassays (RIA), fluorescence assays, chemiluminescent assays, immunoblot assays (Western blots), particulate-based assays, and other known techniques. In some embodiments, antibody-polypeptide complexes are formed in solution. Such complexes can be detected by immunoprecipitation. See, e.g., Short Protocols in Molecular Biology, Chapter 10, Section VI, Ausubel et al., (eds.), Green Publishing Associates and John Wiley & Sons (1992).

### Kits for Detecting Anti-PRRS Virus Antibodies

The PRRS virus polypeptides provided herein can be used to make kits for detecting anti-PRRS virus antibodies. Such kits can contain one, two, three, four, five, six, seven, eight, nine, ten, or more different PRRS virus polypeptides. For example, a kit can contain a PRRS virus NSP 1 polypeptide, a PRRS virus NSP 2 polypeptide, a PRRS virus NSP 4 polypeptide, a PRRS virus ORF 5 polypeptide, a PRRS virus ORF 6 polypeptide, a PRRS virus ORF 7 polypeptide, or any combination thereof. In some embodiments, the kit can contain a PRRS virus NSP 2P polypeptide and an ORF 7 polypeptide.

The kit containing PRRS virus polypeptides can contain other components including, without limitation, packaging materials (e.g., written instructions), indicator molecules (e.g., anti-swine Ig antibodies), buffers, positive control samples (e.g., a sample containing swine anti-PRRS virus antibodies), and negative control samples (e.g., a sample containing swine serum lacking swine anti-PRRS virus antibodies).

### Assessing an Organism's Immunological State

The methods and materials provided herein can be used to determine an organism's immunological state with respect to a virus. Such methods and materials can be used to determine an immunological state in any organism. For example, the immunological state of a pig, dog, cat, bird (e.g., chicken, turkey, or duck), sheep, cow, horse, goat, monkey, or human can be determined using the methods and materials provided herein. In addition, an organism's immunological state with respect to any virus can be determined. For example, an organism's immunological state with respect to a PRRS virus, a circovirus, an influenza virus, a herpes virus, an adenovirus, a parvovirus, a coronavirus, a picornavirus, a parainfluenza virus, or a filovirus can be determined.

In one embodiment, the methods and materials provided herein can be used to determine whether an organism's immunological state is such that (1) the organism received a vaccine version of a virus, (2) the organism was infected with a naturally-occurring version of the virus, or (3) the organism is immunologically naive with respect to the virus. In some cases, the methods and materials provided herein can be used to differentiate between organisms having either an immunological state such that (1) the organism received a vaccine version of a virus or (2) the organism was infected with a naturally-occurring version of the virus.

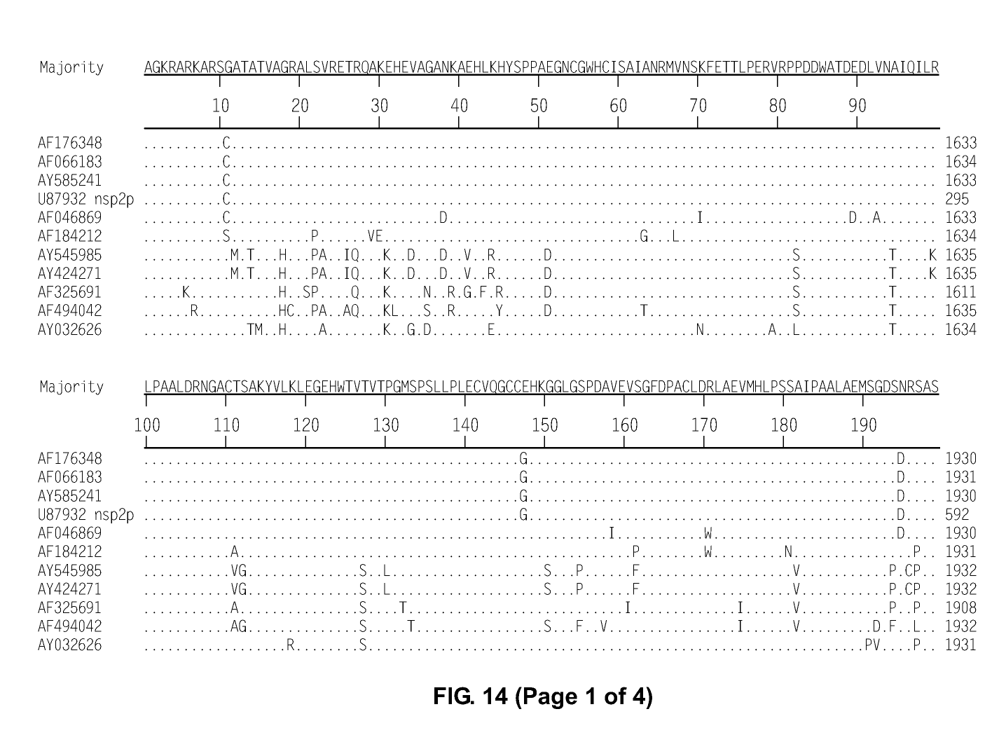

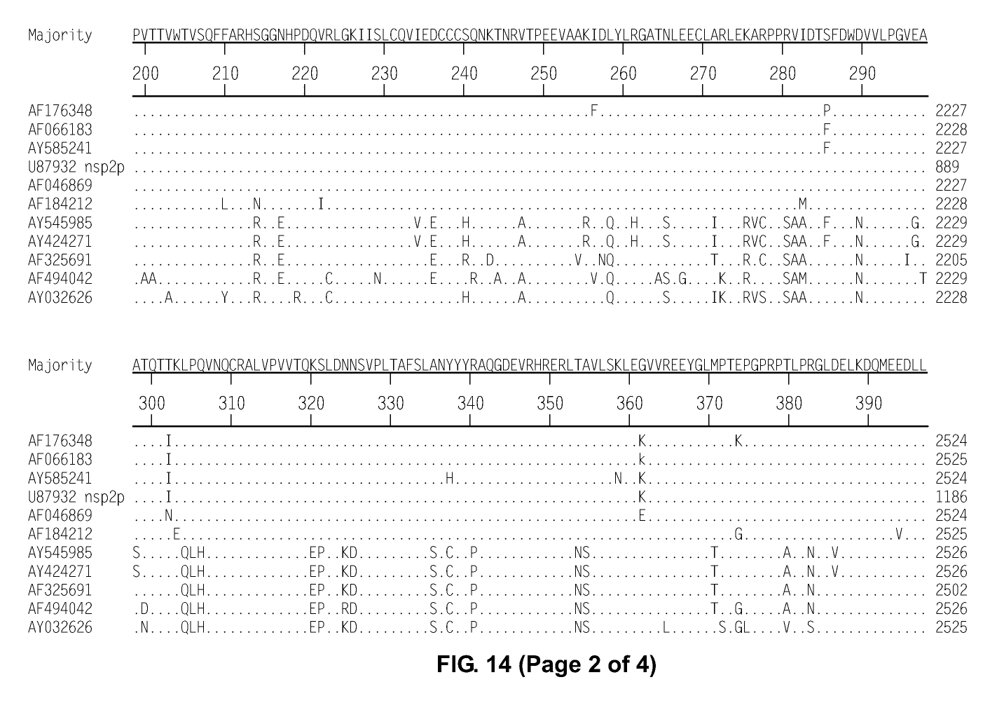

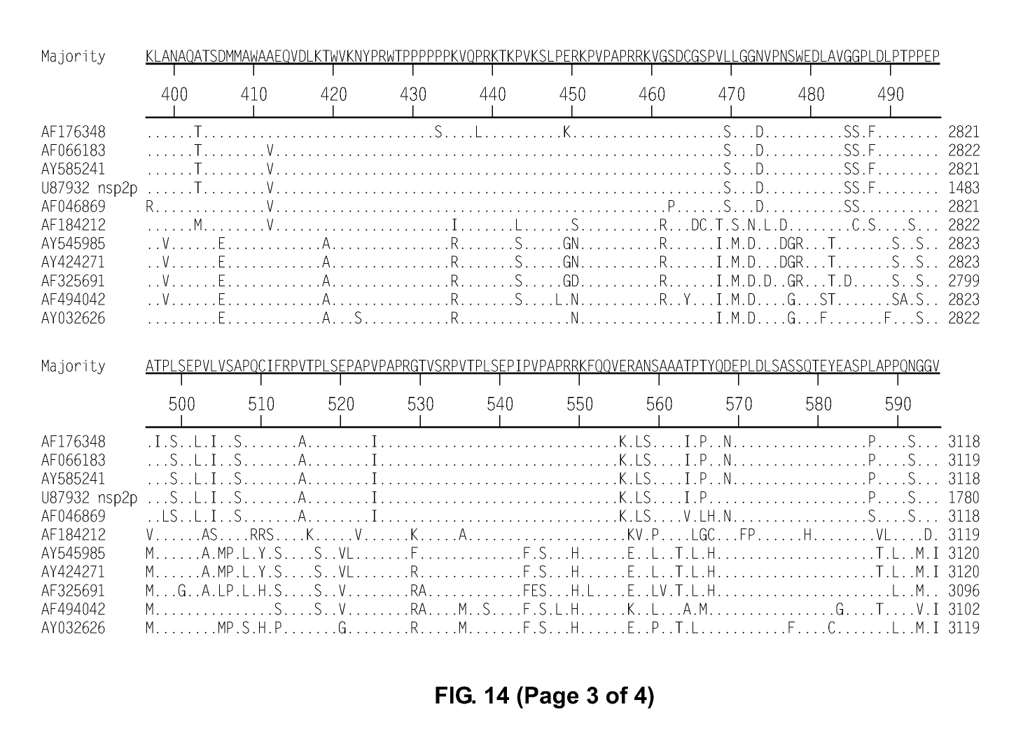

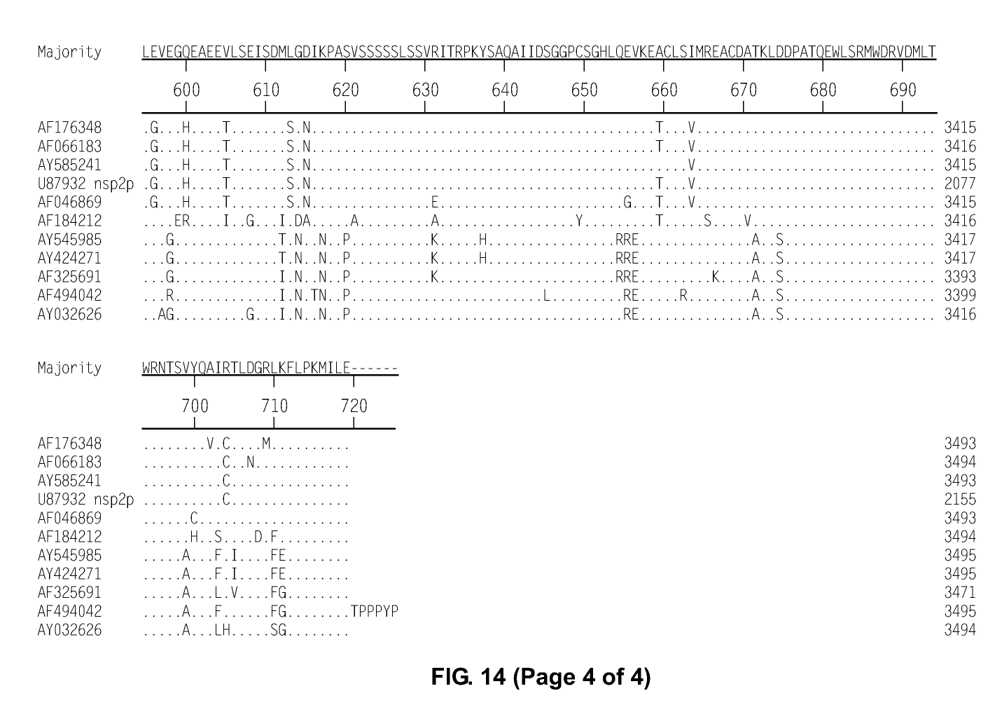

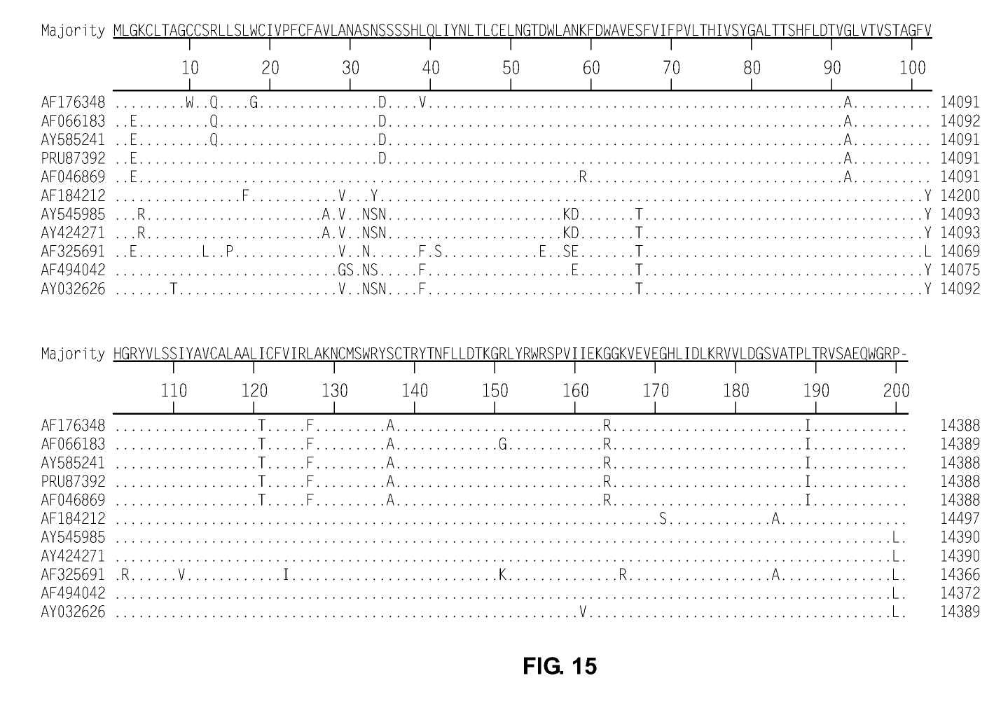

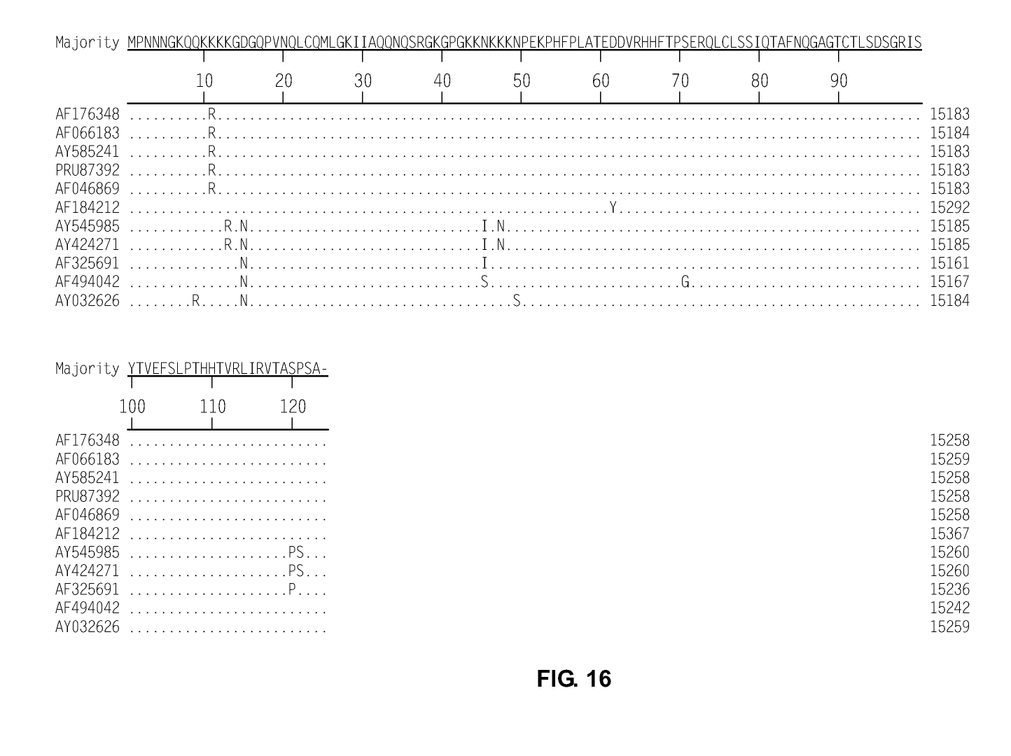

In general, at least two polypeptides are used to assess an organism's immunological state. The first polypeptide can be a polypeptide having an amino acid sequence that is conserved (e.g., highly conserved or, in some cases, completely conserved) between a vaccine version of a virus and naturally-occurring versions of the virus. For example, the first polypeptide can have an amino acid sequence such that antibodies made against a vaccine version of the virus bind the first polypeptide and antibodies made against naturally-occurring versions of the virus bind the first polypeptide. When assessing the immunological state of a pig with respect to a PRRS virus, the first polypeptide can be a polypeptide having an amino acid sequence that is conserved among vaccine and naturally-occurring versions of PRRS viruses such as a C-terminal region of a PRRS ORF 5 polypeptide. Other amino acid sequences conserved among vaccine and naturally-occurring versions of PRRS viruses can be obtained from standard sequence alignments (FIGS. 14-16).

The second polypeptide can be a polypeptide having an amino acid sequence that is not well conserved (e.g., a variable sequence) between a vaccine version of a virus and naturally-occurring versions of the virus. The second polypeptide can have a sequence that is similar or identical to a sequence present in a vaccine version of the virus. For example, the second polypeptide can have an amino acid sequence such that antibodies made against a vaccine version of the virus bind the second polypeptide and antibodies made against naturally-occurring versions of the virus do not bind the second polypeptide. When assessing the immunological state of a pig with respect to a PRRS virus, the second polypeptide can be a polypeptide having an amino acid sequence that is variable among vaccine and naturally-occurring versions of PRRS viruses such as an N-terminal region of a PRRS ORF 5 polypeptide. The amino acid sequence of such a second polypeptide can be from a VR-2332 or RespPRRS PRRS virus. Other amino acid sequences not conserved among vaccine and naturally-occurring versions of PRRS viruses can be obtained from standard sequence alignments (FIGS. 14-16).

To assess an organism's immunological state with respect to a virus, the first and second polypeptides can be contacted with a sample from the organism under conditions such that the first and second polypeptides can bind to anti-virus antibodies, if present within the sample, to form either (1) first polypeptide-antibody complexes or (2) first polypeptide-antibody complexes and second polypeptide-antibody complexes. The formation of first polypeptide-antibody complexes and not second polypeptide-antibody complexes can indicate that the sample is from an organism that was exposed to a naturally-occurring version of the virus. The formation of both first polypeptide-antibody complexes and second polypeptide-antibody complexes can indicate that the sample is from an organism that was exposed to a vaccine version of the virus. The failure to detect either first polypeptide-antibody complexes or second polypeptide-antibody complexes can indicate that the sample is from an organism that is naïve with respect to the virus.

In the case of assessing a pig's immunological state with respect to PRRS virus, the first and second polypeptides can be contacted with a blood sample from the pig under conditions such that the first and second polypeptides can bind to anti-PRRS virus antibodies, if present within the blood sample, to form either (1) first polypeptide-antibody complexes or (2) first polypeptide-antibody complexes and second polypeptide-antibody complexes. The formation of first polypeptide-antibody complexes and not second polypeptide-antibody complexes can indicate that the sample is from a pig that was exposed to a naturally-occurring version of PRRS virus. The formation of both first polypeptide-antibody complexes and second polypeptide-antibody complexes can indicate that the sample is from a pig that was exposed to a vaccine version of PRRS virus. The failure to detect either first polypeptide-antibody complexes or second polypeptide-antibody complexes can indicate that the sample is from a pig that is naïve with respect to the virus.

Typically, the virus polypeptides are immobilized on solid substrates such as dipsticks, microtiter plates, particles (e.g., beads), affinity columns, and immunoblot membranes. See, U.S. Pat. Nos. 5,143,825; 5,374,530; 4,908,305; and 5,498,551 for exemplary descriptions of solid substrates and methods for their use. For example, PRRS virus polypeptides (e.g., one polypeptide with a PRRS virus sequence limited to a conserved PRRS virus amino acid sequence and another polypeptide with a PRRS virus sequence limited to a divergent PRRS virus amino acid sequence) can be immobilized on a solid substrate, such as a 96-well plate, using known methodologies, then contacted with a sample for a pig under conditions such that anti-PRRS virus antibodies present within the sample can bind to the immobilized PRRS virus polypeptides to form polypeptide-antibody complexes. Suitable conditions include incubation in an appropriate buffer (e.g., sodium phosphate buffer, pH 7.2 to 7.4) at room temperature from about at least 10 minutes to about 10 hours (e.g., from about 1 to about 2.5 hours). Thereafter, unbound material is washed away, and polypeptide-antibody complexes can be detected as described herein.

### Kits for Assessing an Organism's Immunological State

A first polypeptide having an amino acid sequence such that antibodies made against a vaccine version of the virus can bind that first polypeptide and antibodies made against naturally-occurring versions of the virus can bind that first polypeptide can be combined with a second polypeptide to make a kit for assessing an organism's immunological state. The second polypeptide can have a sequence that is similar or identical to a sequence present in a vaccine version of the virus. In addition, the second polypeptide can have an amino acid sequence such that antibodies made against a vaccine version of the virus bind that second polypeptide and antibodies made against naturally-occurring versions of the virus do not bind that second polypeptide. Such kits can contain additional polypeptides. For example, a kit can contain two, three, four, five, six, seven, eight, nine, ten, or more different polypeptides with each having a different sequence that is conserved among vaccine and naturally-occurring versions of the virus. Likewise, a kit can contain two, three, four, five, six, seven, eight, nine, ten, or more different polypeptides with each having a different viral sequence that is not conserved among vaccine and naturally-occurring versions of the virus.

The kit can contain other components including, without limitation, packaging materials (e.g., written instructions), indicator molecules (e.g., anti-organism Ig antibodies), buffers, positive control samples, and negative control samples.

In some cases, a solid support can be contacted with a polypeptide and a lysozyme to increase the ability of the polypeptide attached to the solid support to react with an antibody that binds the polypeptide. Any polypeptide can be attached to a solid support including, without limitation, the PRRS virus polypeptides provided herein (e.g., a PRRS virus ORF 7 polypeptide). Any lysozyme can be used. Typically, a lysozyme can be a hydrolytic enzyme that degrades β-1,4 glucosidic linkages between N-acetylmuramic acid and N-acetylglucosamine in cell walls of certain bacteria, particularly Gram-positive bacteria. A lysozyme can be found in animal secretions and tissues, including, without limitation, saliva, tears, milk, urine, cervical mucus, leucocytes, and kidneys. For example, a lysozyme can be found in uterine secretions of the pig (Roberts and Bazer, J. Reprod. Fertil., 82:875-892 (1988)). Lysozyme from chicken egg white has been extensively studied, and was the first enzyme for which a crystal structure was solved (Diamond, J. Mol. Biol., 82:371-391 (1974)). Lysozyme is widely distributed in egg white of birds (Prager et al., J. Biol. Chem., 249:7295-7297 (1974)). Structure-function relationships of lysozymes are described elsewhere (Imoto et al., J. Vertebrate Lysozymes, The Enzymes 7, P. Boyer, Academic Press, NY, 1972)).

Any ratio of polypeptide to lysozyme can be used. For example, a polypeptide and a lysozyme can be contacted with a solid support at a ratio of at least 4 ng of the polypeptide per 1 ng of the lysozyme (e.g., 4:1, 5:1, 6:1, 7:1, or more). In some cases, a lysozyme and a polypeptide can be contacted with a solid support at a ratio of at least 1 ng of the lysozyme per 1 ng of the polypeptide (e.g., 1:1, 2:1, 3:1, 4:1, 5:1, or more).

The solid support can be any type of solid support including, without limitation, glass slides, plastic plates, 96-well plates, beads, and the like.

The invention will be further described in the following examples, which do not limit the scope of the invention described in the claims.

### EXAMPLES

### Example 1

### Production of Recombinant PRRS Virus Polypeptides

### Methods and Materials

Plasmid cloning vectors pET24b, pET25b, and pETBlue2 were obtained from Novagen (Madison, Wis.) and pGEM-T was obtained from Promega (Madison, Wis.). E. coli BL21(DE3) cells were obtained from Novagen. E. coli BL21(DE3) strains Tuner, RP, and ABLE-K were obtained from Stratagene (La Jolla, Calif.). DH5α cells were obtained from Invitrogen (Carlsbad, Calif.). Plasmid and DNA purification kits were obtained from Qiagen (Valencia, Calif.). PCR reagents were obtained from Applied Biosystems (Roche Molecular Systems, Branchburg, N.J.). Standard lab supplies, bacterial growth media, and electrophoresis chemicals were obtained from Sigma Chemical Co. (St. Louis, Mo.). PRRS virus cDNA fragments for cloning were obtained by reverse transcriptase-PCR amplification of regions of VR-2332 genomic RNA encoding NSP 1α and 1β, NSP 2, and NSP 4.

### PCR Amplification, Cloning of DNA Fragments, and Restriction Analysis

Primers for PCR were designed using Primer3 (Whitehead Institute for Biomedical Research, Cambridge, Mass.) and PRRS virus strain VR2332 sequence (GenBank accession number U87392 or PRU87392) (Table 1). Primers were synthesized, purified, and quantified by Integrated DNA Technologies, Inc. (Coralville, Iowa). PCR reactions used the Applied Biosystems heat activated AmpliTaq Gold® kit (Roche Molecular Systems, Branchburg, N.J.). The reaction mixtures (50 μL total volume) contained 10× Buffer II (1× concentration), 1.5 mM MgCl₂, 200 μM each of dATP, dCTP, dGTP, dTTP; 0.2 μM each primer pair (Table 1); 1.0 U AmpliTaq Gold®, and the appropriate cDNA. Upon mixing, the solutions were immediately placed in the thermocycler (GeneAmp PCR system 2400, Perkin Elmer, Shelton, Conn.). Temperature cycle: 1 cycle (95° C. for 10 minutes); 35 cycles (94° C. for 30 seconds, 55° C. for 30 seconds, 72° C. for 45 seconds); 1 cycle (72° C. for 7 minutes, 4° C. hold). The resulting amplified DNA was then separated using an agarose gel. Bands corresponding to the predicted product sizes were gel extracted (Gel Extraction Kit, Qiagen) and then further purified using a PCR Purification Kit. The isolated products were then cloned into pGEM-T vector and transformed into DH5α cells, which were spread on LB 100 μg/mL ampicillin (Amp) agar plates with IPTG and X-Gal. White colonies were selected and grown. The nucleic acid from the selected colonies was sequenced using the standard T7 and SP6 primers (Advanced Genetic Analysis Center, University of Minnesota, St, Paul Minn.). After an initial BLAST search screening (GeneBank NCBI, Bethesda, Md.), trace files were edited to remove vector sequence (Seqman, DNASTAR, Inc., Madison, Wis.) and aligned (Megalign, DNASTAR).

A specialized vector based on pET 24b (Novagen, Madison, Wis.) containing a myc tag 5′ leader sequence and a terminal 3′ His tag was engineered for high efficiency polypeptide expression and isolation. This plasmid (pET 24b myc H is) contains a Bam HI site immediately 3′ to the myc tag and a Xho I site preceding the terminal 6×His tag. The vector was prepared for insertion by digestion with BamHI and Xho I, followed by dephosphorylation with calf intestinal alkaline phosphatase (CIAP) (Promega). PCR conditions, insert isolation, and purification were as described above followed by restriction digestion (BamHI, Xho I) to prepare the insert for ligation. Ligation reactions typically contained 100 ng of dephosphorylated vector, 20 ng insert, 1× ligation buffer, and 400 Units T4 ligase (New England Biolabs, Beverly, Mass.), total volume 10 μL. The ligation reaction was placed at 16° C. for 16 hours before transformation into DH5α cells. Colonies were selected as previously described and grown. The nucleic acid from the selected colonies was sequenced and analyzed (yielding plasmid pET 24b myc-polypeptide-His).

### Test Protein Expression

To test polypeptide expression, recombinant plasmids were transformed into BL21 (DE3)-RP cells, which contain eukaryotic tRNA's for arginine and proline and are chloramphenicol (cam) resistant. Transformed cells were spread on kanamycin 30 μg/mL (kan 30), chloramphenicol 35 μg/mL (cam 35) LB plates and screened by colony PCR using the T7 and SP6 primers for the pET 24b plasmid. Ten positive colonies were grown overnight at 30° C. in 2 mL of 2xYT media (kan 30, cam 35). 200 μL of each of the overnight cultures were used to inoculate ten temperature equilibrated (30° C.) 10 mL aliquots of 2xYT (kan 30). These cultures were grown at 30° C. to an OD₆₀₀ of 0.4, 200 μL was remove for SDS-PAGE analysis, and IPTG was added to a final concentration of 1.0 mM. The induced samples were allowed to grow at 30° C. for 4 hours, and then 200 μL were removed for SDS-PAGE analysis.

### Large Scale Polypeptide Expression and Purification

Polypeptides were purified using a modification of the Qiagen Ni-NTA agarose affinity isolation procedure for native His tagged proteins. Briefly, the induced bacterial cells from a 1-liter culture were pelleted at 4000 g for 20 minutes at 4° C., and supernatant was decanted. The pellet was resuspended in 30 mL of lysis buffer (50 mM NaH₂PO₄, 300 mM NaCl, 10 mM imidazole, 1 μM pepstatin A, 1 μM leupeptin, and 1 mM PMSF, at pH 8.0), and then lysozyme was added to a final concentration of 1.0 mg/mL. The solution was incubated on ice for 60 minutes, followed by sonication on ice using six 10-second bursts of 250 W at 10-second intervals. RNAse A (10 μg/mL final) and DNAse I (5 μg/mL final) were then added, and the solution was incubated on ice for an additional 15 minutes to further degrade nucleic acids. The lysate was then centrifuged (4° C.) for 30 minutes at 10,000×g to pellet the insoluble aggregates and cellular debris. The pellet contained the majority of expressed recombinant polypeptide in the form of inclusion bodies and was isolated in the denatured form to be refolded later. Immediately following centrifugation, this pellet was resuspended in 30 mL of a solution containing 100 mM NaH₂PO₄, 10 mM Tris-HCl, and 8 M urea, at pH 8.0. The resuspended pellet was rotated (200 rpm) at room temperature for 30 minutes and then placed at 4° C. for later processing.

The supernatant containing various levels of soluble polypeptide was decanted into 6 mL of 50% Ni-NTA slurry and gently rotated (200 rpm) for 1 hour at 4° C. The supernatant-Ni-NTA mixture was then poured into a 1.5×30 cm column and drained by gravity. The column was washed twice with 20 mL of a solution containing 50 mM NaH₂PO₄, 300 mM NaCl, 20 mM imidazole, 1 μM pepstatin A, 1 μM leupeptin, and 1 mM PMSF at pH 8.0. The polypeptide was eluted with four 3-mL aliquots of elution buffer containing 50 mM NaH₂PO₄, 300 mM NaCl, 250 mM imidazole, 1 μM pepstatin A, 1 μM leupeptin, and 1 mM PMSF at pH 8.0. Purified polypeptides were concentrated by either tangential flow filtration cassette (Pellicon XL Ultracel PLC 5 kD, Millipore, Bedford Mass.) or a YM-3 Amicon Centriprep® centrifugal filter device (Millipore Corp. Bedford, Mass.), followed by dialysis (Spectra/Por MWCO® 6-8,000, Spectrum Laboratories, Rancho Dominguez, Calif.) against 50% glycerol and 20 mM Tris HCl, pH 7.5. Polypeptide concentrations were determined using the Bio-Rad RC DC protein assay kit (Bio-Rad, Hercules, Calif.). Purified polypeptide solutions were stored at −20° C.

The denatured insoluble recombinant polypeptide mixture stored at 4° C. was centrifuged at 4° C. for 30 minutes at 10,000×g to pellet cellular debris. The supernatant contained high levels of previously insoluble denatured recombinant polypeptide and was decanted into 6 mL of 50% Ni-NTA slurry and gently rotated (200 rpm) for 1 hour at 4° C. The supernatant-Ni-NTA mixture was then poured into a 1.5×30 cm column and allowed to drain. The column was washed twice with 20 mL of a solution containing 100 mM NaH₂PO₄, 10 mM Tris-HCl, and 8 M urea, at pH 6.3. The polypeptide was then eluted 4 times with 3 mL aliquots of elution buffer containing 100 mM NaH₂PO₄, 10 mM Tris-HCl, and 8 M urea, pH 5.9. SDS gel analysis, concentration of the polypeptide, dialysis into PBS, and the concentration determinations were done as described above.

### Polypeptide Refolding

Refolding of the denatured recombinant polypeptide was performed using a variation of the methods described elsewhere (Buchner et al., Anal. Biochem., 205:263-270 (1992) and Clark, Curr. Opin. Biotechnol., 9:157-163 (1998)). Briefly, denatured polypeptide solutions containing purified polypeptide were pooled and dialyzed (Spectra/Por MWCO® 6-8,000, Spectrum Laboratories, Rancho Dominguez, Calif.) for 4 hours at 4° C. against 500 mL of 0.1 M Tris, pH 8.0, 6 M guanidine-HCl, and 2 mM EDTA. The dialysis was then repeated with fresh buffer for an additional 4 hours. After adjusting the polypeptide concentration to 3 mg/mL (concentration determined with the Bio-Rad RC DC protein assay kit, Bio-Rad, Hercules, Calif.), dithiothreitol (DTT) was added to a final concentration of 300 mM DTT. The resulting 5-mL solution was stirred at room temperature for 2 hours followed by filtration using a 0.45 μm filter (Syringe Filter, Fisher Scientific, Pittsburgh, Pa.). The reduced polypeptide solution was then added rapidly at 4° C. with moderate stirring into 500 mL of refolding buffer (100 mM Tris HCl, pH 8.0, 0.5 M L-arginine, 8 mM oxidized glutathione, 2 mM EDTA, 10 μM pepstatin A, 10 μM leupeptin, and 1 mM PMSF) corresponding to a final dilution of about 1:100. The resulting solution was then filtered through a 0.22 μm membrane (Steritop, Millipore, Bedford Mass.) to remove particulates and stirred overnight. Purified polypeptide was concentrated by tangential flow filtration cassette (Pellicon XL Ultracel PLC 5 kD, Millipore, Bedford Mass.) to a volume of 10 mL followed by dialysis (Spectra/Por MWCO® 6-8,000, Spectrum Laboratories) against 50% glycerol and 20 mM Tris HCl, pH 7.5. Polypeptide concentrations were determined using the Bio-Rad RC DC protein assay kit (Bio-Rad, Hercules, Calif.). Purified polypeptide solutions were stored at −20° C.

### Gel Electrophoresis and Immunoblotting

Bacterial lysates, purification fractions, and purified polypeptides were analyzed on SDS-polyacrylamide gels with the Laemmli buffer system (Laemmli, Nature, 227:680-684 (1974). Protein bands were visualized by staining with 0.025% Coomassie blue. For immunoblotting, gels were electroblotted onto supported nitrocellulose membranes (MSI Separations, Westbrook Mass.). Membranes were incubated with anti-myc monoclonal antibody 9E10 for 1 hour at room temperature. Antibody binding was detected using alkaline phosphatase-conjugated goat-anti-mouse IgG and visualized with the ECL Western Blotting system (Amersham Pharmaciea Biotech, Piscataway, N.J.).

### ELISA

ELISA plates were coated with individual PRRS virus polypeptides in 100 μL carbonate buffer (15 mM Na₂CO₃ and 35 mM NaHCO₃), pH 9.6, or buffer alone overnight and washed 6 times with PBS-Tween (0.1% Tween-20). Two hundred μL of PBS-Tween containing 2.5% nonfat dried milk was added for 1 hour at room temperature to block previously unbound sites, and the plates were washed 5 times. One hundred μL of pig serum at various dilutions was added in duplicate for 2 hours at room temperature, and plates were washed 4 times with PBS-Tween. Levels of specific antibody were determined by incubation of wells in horseradish peroxidase-conjugated goat-anti swine IgG (heavy+light chains) (KPL, Gaithersburg Md.) diluted 1:5000 for 1 hour. Wells were washed 5 times, and color was developed with 100 μL of TMB substrate (KPL). Reactions were stopped after 15 minutes with 100 μL 1 M phosphoric acid, and the plates read at 450 nm.

pETBlue2 (Novagen)

Clones in pGEM-T were transformed into DH5α cells. Colonies were grown overnight, and plasmids were isolated with Qiagen Miniprep Purification Kits. The purified plasmids and 2 μg of pETBlue2 were digested individually with Nco I and Not I for 4 hours. pETBlue2 was dephosphorylated with CIAP for the last 20 minutes of digestion. Insert fragments and linearized pETBlue2 were gel purified with the Qiagen Gel Purification Kit and then ligated in an about 1:2 ratio of vector to insert.

The ligations were transformed into DH5α cells. Two colonies per plate were cultured overnight, and a plasmid preparation was performed on the cultures to isolate the plasmids. The purified plasmids were then transformed into BL-21 (DE3) RP cells. Two colonies per clone were cultured and induced with 400 μM of IPTG for 4 hours at 30° C. A subsequent SDS-PAGE gel of the whole cell lysates showed no evidence of specific polypeptide induction. Similarly, ELISA tests of induced cell lysates coated on microtiter plates and reacted with PRRS⁺ and PRRS swine sera did not reveal evidence of PRRS virus polypeptide. Evaluation of pETBlue2 for PRRS virus polypeptide expression was stopped at this point.

pET24d/pET25b (Novagen)

2 μg of pET24d was digested with Nco I and Not I and dephosphorylated with CIAP. The fragment was then purified with a Qiagen PCR Purification Kit. An agarose gel was used to further purify the fragment, and a QIAquick Gel Extraction Kit was used to extract the vector fragment from the gel. The pET24d fragment was then ligated to the clone fragments in an about 2:1 insert to vector ratio. Transformation of these plasmids into DH5α cells did not result in colony growth. Similar results were obtained after cloning into pET24d vector that was not dephosphorylated or into pET25d that was or was not dephosphorylated.

### Cloning

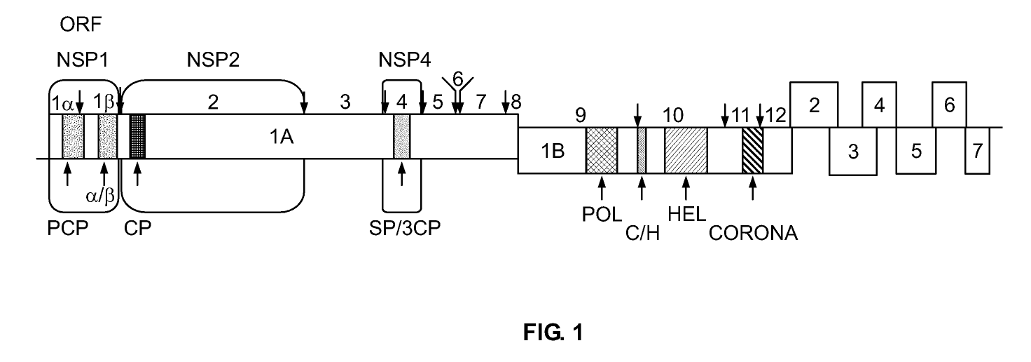

PCR was used to amplify the three PRRS virus proteases that were identified by their active sites, at the following amino acid positions in ORF 1a of PRRS virus strain VR2332: papain-like cysteine protease α and β (PCP α/β) (amino acids 74-146 and 268-339), unusual cysteine protease (amino acids 435-506), and the poliovirus 3C-like serine protease (amino acids 1840-1946). The location of these functional protease domains in the PRRS virus genome is shown graphically in FIG. 1. Table 1 lists the nucleotide sequence regions of PRRS virus strain VR2332 that were PCR amplified and summarizes the overall results.

TABLE 1
PRRS virus NSP fragments cloned.

| Nonstructural | Region | Restriction | |
|---|---|---|---|
| protein (NSP) | Amplified | Sites | Results |
| 1 (PCPα/β) | 174-1322 | AccIII, BamHI | PCR band, digestion |
| | | NdeI, XhoI | product, transformed |
| | | XhoI, NcoI | bacterial colonies positive |
| | | | by PCR screening, no |
| | | | plasmid |
| 2 (unusual | 1339-4922 | BamHI, NcoI | PCR band, digestion |
| cysteine | | NdeI, XhoI | product, PCR-positive |
| protease) | | | colonies, no plasmid |
| 4 (poliovirus | 5598-6209 | NdeI, XhoI | PCR band, digestion |
| 3C-like serine | | | product, transformed |
| protease) | | | bacterial colony, plasmid |
| | | | with insert, point mutation |
| | | | in protein |

### Nonstructural Protein 1 (NSP 1)

Ligation products of this fragment and pET24b yielded colonies following transformation of E. coli DH5α cells. Colonies grew slowly and typically required 48 hours at 37° C. to be visible. Screening of colonies by PCR gave positive results consistent with the presence of a cloned fragment. Efforts to recover recombinant plasmid from bacteria grown in broth were unsuccessful. It appeared that recombinant plasmids were unstable. To overcome this problem, a variety of E. coli strains were used as plasmid recipients: DH5α, JM109, HB101, SURE (Stratagene), and ABLE (Stratagene). Transformation plates and broth cultures were incubated at 37°, 30°, and 22° C. Culture volumes of 1, 2, 5, 10, and 25 mL were performed. Various methods of plasmid purification were attempted, including Qiagen miniprep, standard alkaline lysis with phenol/chloroform extraction, and boiling lysis with lithium chloride/isopropanol precipitation. None of these conditions and treatments resulted in the recovery of recombinant plasmid. In all, 353 transformants were screened by colony PCR, with about 70 reactions yielding bands. Plasmid purifications yielded no visible bands or a high molecular weight band, which upon diagnostic restriction digestion disappeared from the gel. This result is consistent with the behavior of genomic DNA.

### Nonstructural Protein 2 (NSP 2)

The same results were obtained as with NSP 1. Transformed colonies were obtained on LB agar plates that were positive by PCR, but attempts to isolate plasmid DNA were unsuccessful.

### Nonstructural Protein 4 (NSP 4)

The results with NSP 4 were identical to the experiences with NSP 1 and NSP 2 with one exception. Plasmid DNA was successfully recovered from a clone and was shown by DNA sequencing to contain the predicted NSP 4. A point mutation was noted that changed amino acid 16 from isoleucine to threonine.

Cloning of NSP Fragments in pET24bmycHis

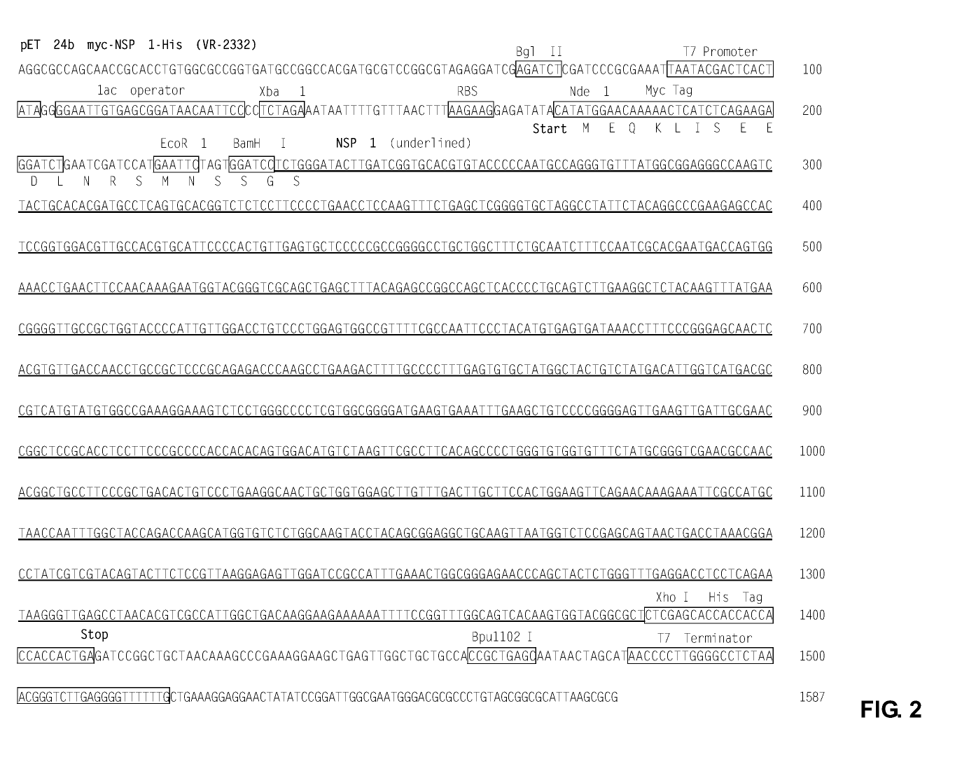

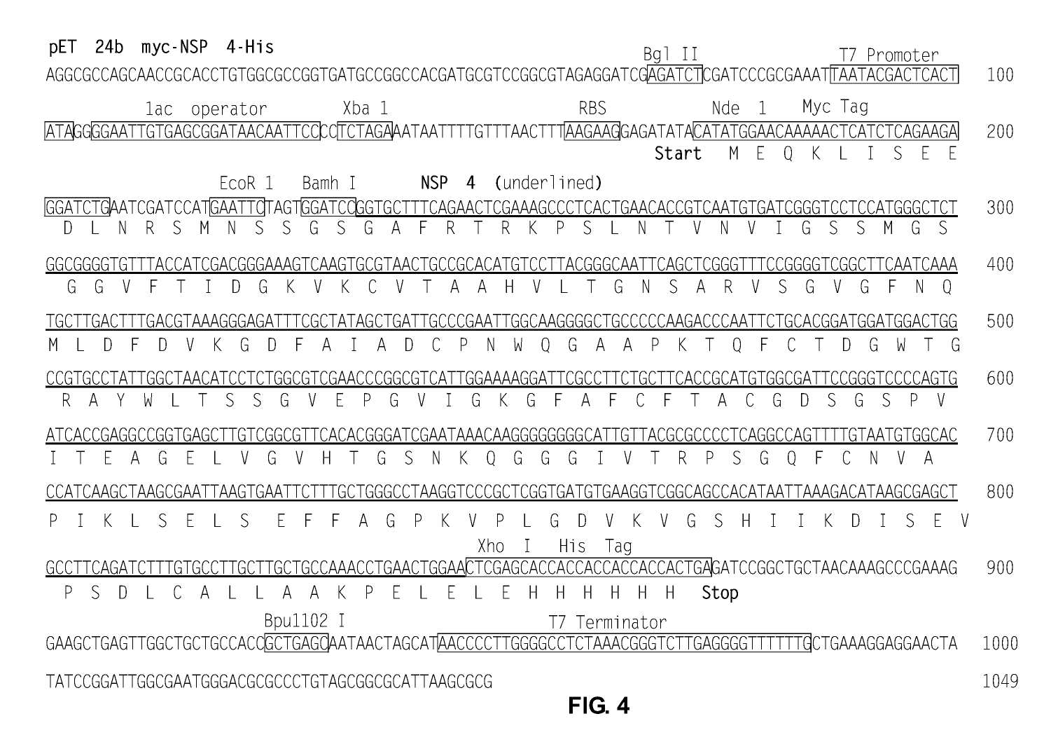

DNA fragments corresponding to NSP 1, NSP 2, and NSP 4 were amplified by PCR and cloned into pET24b-mycHis (FIGS. 2, 3, and 4). Functionally positive clones were identified by small-scale test induction of individual colonies, and a single, high expressing clone was picked, grown, purified, and sequenced. Each clone contained EcoR1 and BamHI sites at the 5′-end and an XhoI site at the 3′-end. The encoded polypeptides contained an amino terminal myc tag and a carboxyl terminal 6×His tag.

### Recombinant NSP Expression and Purification

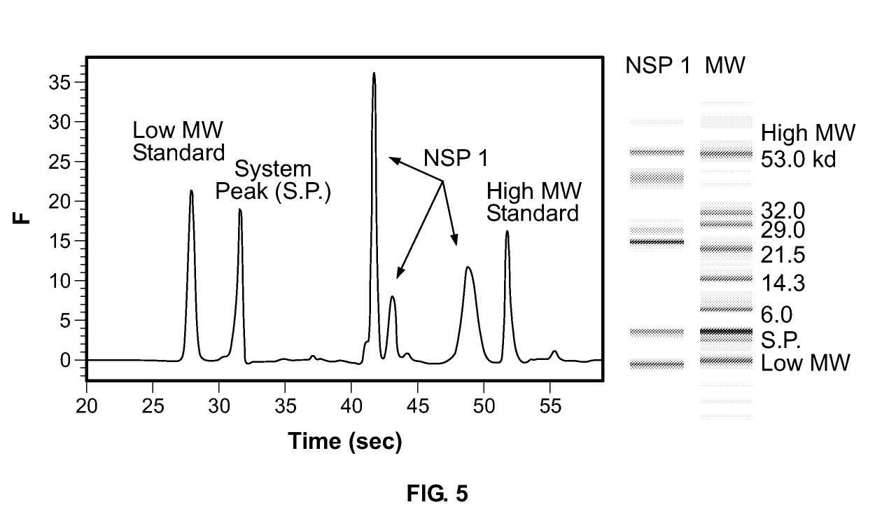

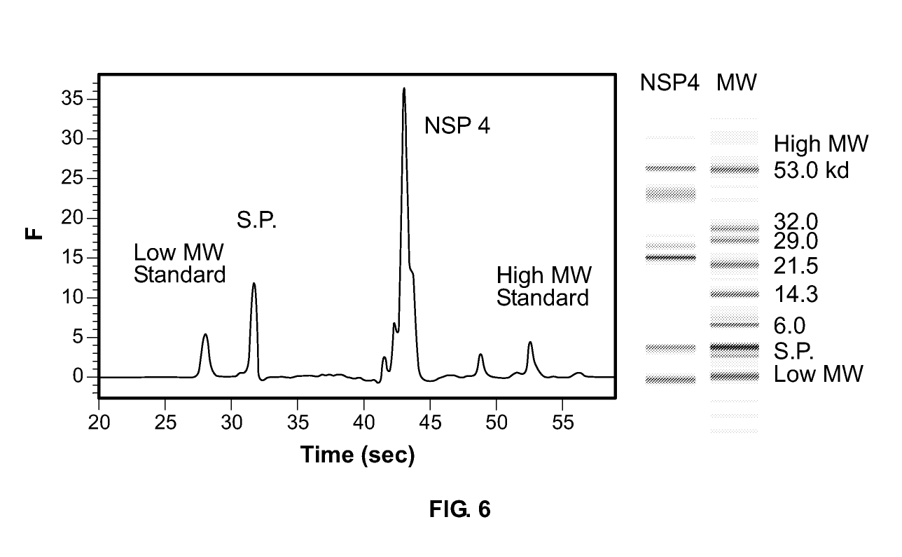

Individual colonies were grown and induced for polypeptide expression as described herein. Polypeptides were purified by Ni-NTA immobilized metal affinity chromatography. Recombinant NSP 1 and NSP 4 were readily expressed at mg/L levels in shake flasks under the described conditions, and about 50% of the polypeptide was recovered following affinity chromatography and refolding (Table 2). The purified and refolded polypeptides were homogeneous and contained fragment sizes consistent with predicted protease activities. The NSP 1 and NSP 4 polypeptides consisted of homogeneous polypeptides in which the NSP 1 preparation contain intact polypeptide and two fragments autoproteolytically cleaved into PCP 1α and PCP 1β, whereas the NSP 4 preparation was a single band (FIGS. 5 and 6).

TABLE 2
Polypeptide expression yields.

| Nonstructural | Total expressed | Ni-NTA purified | After Refolding |
|---|---|---|---|
| protein (NSP) | (mg/L culture) | (mg/L culture) | (mg/L culture) |
| NSP 1 (pcpα/pcpβ) | 20 | 10 | 9 |
| NSP 4 | 25 | 14 | 13 |

The NSP 2 polypeptide was expressed at low levels that could not be visualized in whole cell lysates on SDS polyacrylamide gels stained with Coomassie blue, but it was observed by western blot detection with anti-myc antibody. The presence of multiple bands at sizes lower than the encoded polypeptide sequence of 132 kD indicated that proteolytic degradation had occurred either during bacterial growth and polypeptide expression or during cell lysis and sample handling. Further evidence that the western blot band contained PRRS virus NSP 2 was obtained from test ELISA results in which microtiter plate wells were coated with induced bacterial lysates from clones expressing NSP 1, NSP 2, or NSP 4. Wells containing NSP 2 polypeptide reacted strongly and in a specific and dilution-dependent fashion. The low level of expression of NSP 2 polypeptide may be due to the presence of a hydrophobic region toward the carboxyl end of the polypeptide.

### Effect of Refolding on ELISA Reactivity

Apparent differences in antibody reactivity among the three NSP polypeptides were observed in the preliminary test ELISA, raising the possibility that the conformation of the purified, recombinant polypeptides might be variable and might affect immunoreactivity. Recombinant nucleocapsid (N) varied in immunoreactivity depending on the conditions of expression, purification, and refolding. Therefore, the immunoreactivity of NSP 1 and NSP 4 was evaluated before and after refolding.

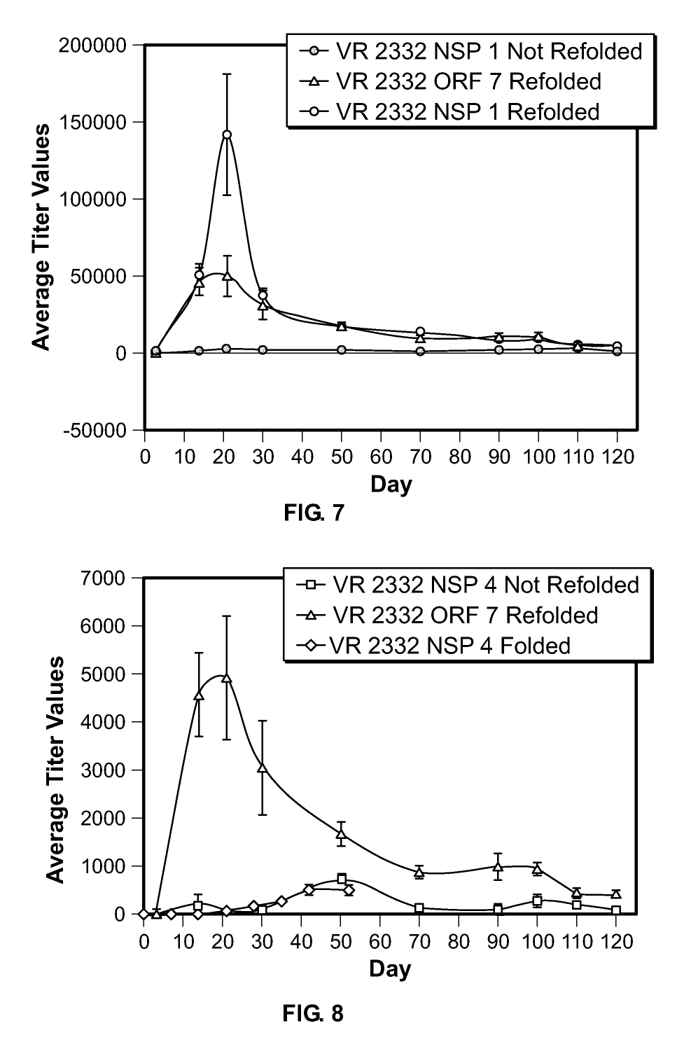

Refolding had an effect on the immunoreactivity of NSP 1 (FIG. 7). Affinity purified NSP 1 polypeptide that was not refolded was essentially non-reactive to serum obtained from pigs during a 120 day period after PRRS virus infection.

By contrast, there was no substantial difference in anti-NSP 4 antibody titers against non-refolded or refolded NSP 4 polypeptides (FIG. 8). The analysis of refolded polypeptide reactivity was terminated at 52 days since it was apparent that there was no difference in the two forms for NSP 4. The lack of effect of refolding was further emphasized by the choice of serum samples for analysis. The maximum antibody response was predicted to occur in animals immunized with homologous virus (VR2332 is the parental strain to Ingelvac MLV vaccine) and tested with refolded, presumably native, polypeptide. The minimum response was predicted to occur in animals infected with a heterologous strain (MN30100) and tested with non-refolded polypeptide. Under these conditions, no differences were observed.

These results demonstrate that polypeptide refolding affects immunoreactivity in the case of NSP 1 and is insignificant for NSP 4. Each recombinant NSP polypeptide, however, is routinely refolded and stored in soluble form in glycerol to maintain a uniform product.

### Induction and Duration of Antibody Responses to NSP Recombinant Polypeptide

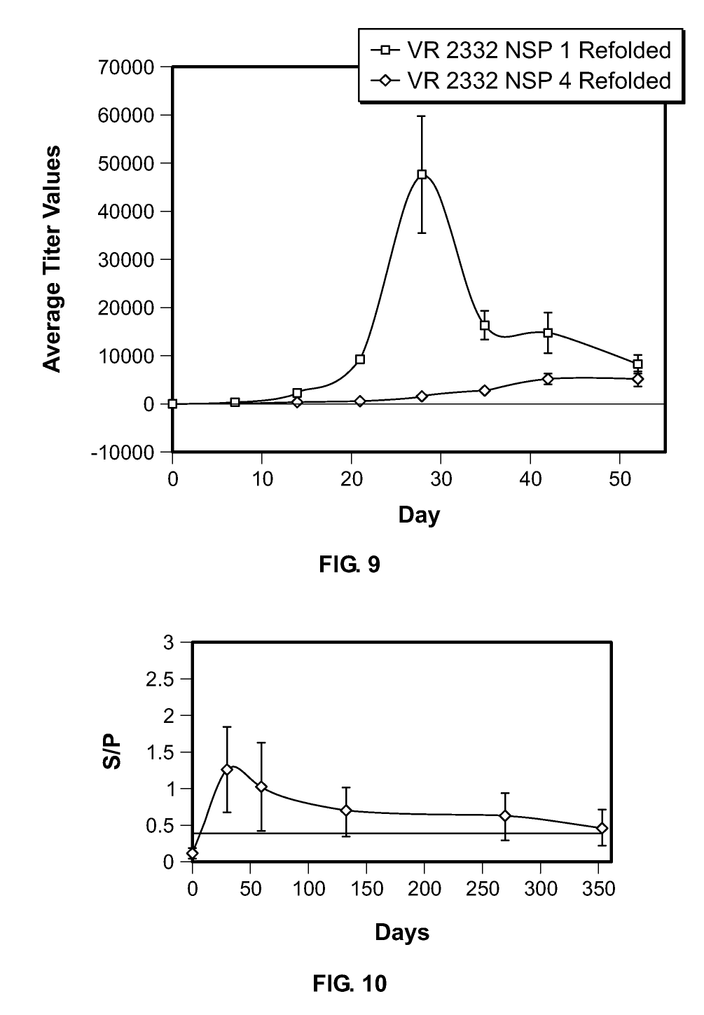

The kinetics of anti-NSP 1 antibody response were similar to the response to N in 4-month old gilts. The anti-NSP 1 titer was about 1/50,000 at 14 days after infection and peaked at 21 days after infection at about 1/140,000. Antibody levels declined rapidly and were equivalent to N(ORF 7) from 28-120 days after infection. In a small group of young pigs (4-6 weeks of age) immunized with Ingelvac MLV, antibody titers to NSP 1 showed a similar sharp peak and rapid decline, but the peak occurred at 28 days instead of 21 days after infection (FIG. 9).

In gilts, the antibody response to NSP 4 was weak in comparison to N and to NSP 1. There was evidence of an increase in titer at 40-55 days after infection, and again, possibly at 100-110 days after infection. In young pigs, there was a similar late and modest increase in anti-NSP 4 titers starting at about 28-35 days after immunization (FIG. 9). This time frame corresponds to the period in which acute infection is resolved.

### Cross-Reactivity of Swine Anti-NSP Antibodies to VR2332NSP Recombinant Polypeptide

Purified and refolded NSP 1 and 4 polypeptides, derived from the VR2332 strain of PRRS virus and expressed in bacteria, reacted equivalently with antiserum from pigs exposed to a homologous strain (Ingelvac MLV) and a heterologous strain (MN30100) of PRRS virus.

### Example 2

### Production of Additional Recombinant PRRS Virus Polypeptides

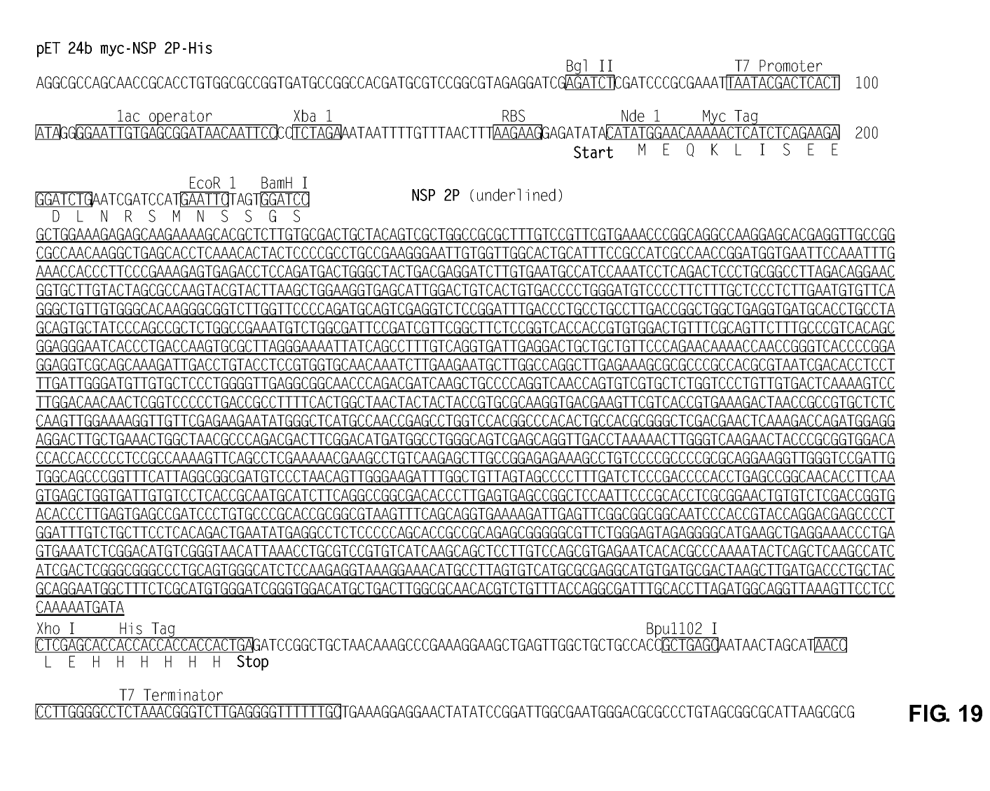

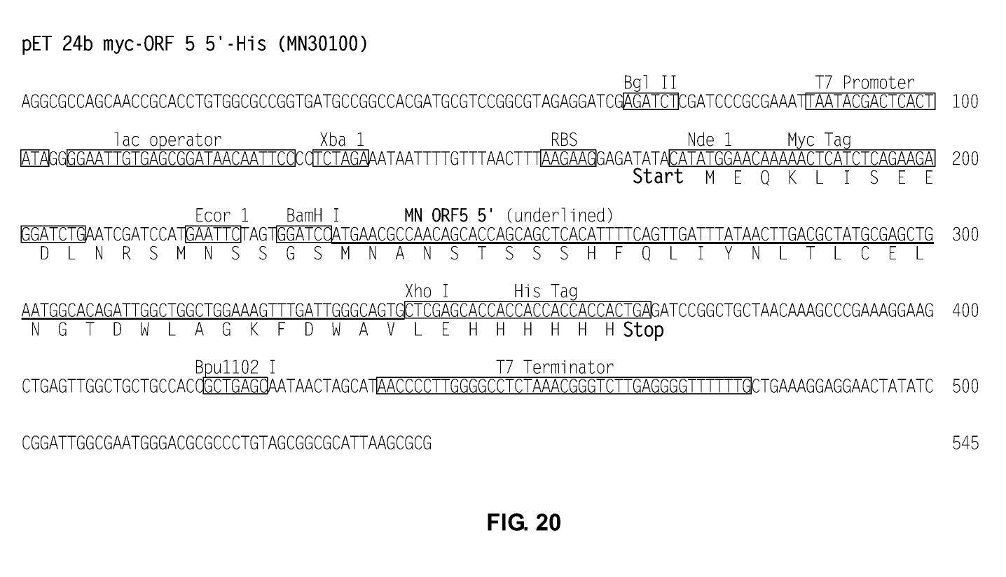

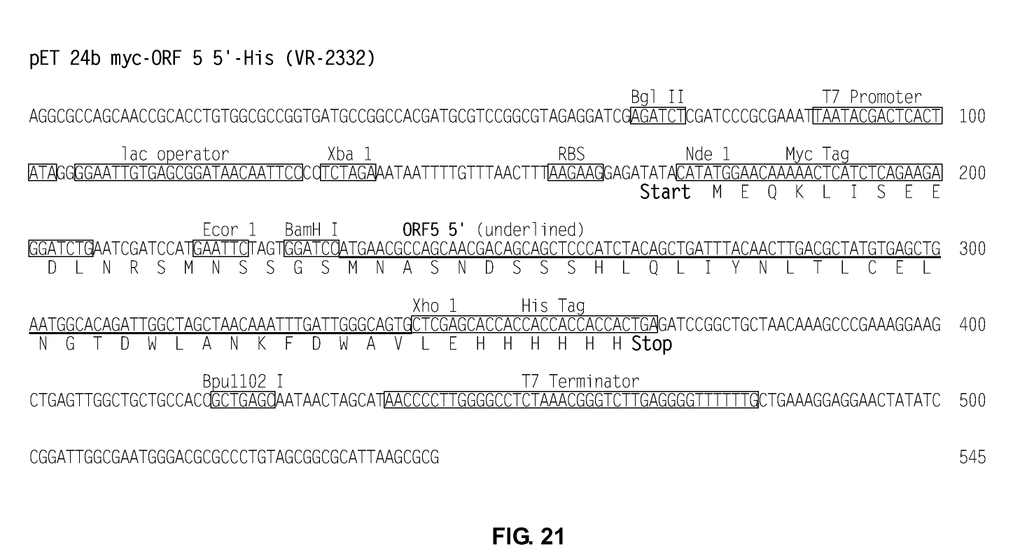

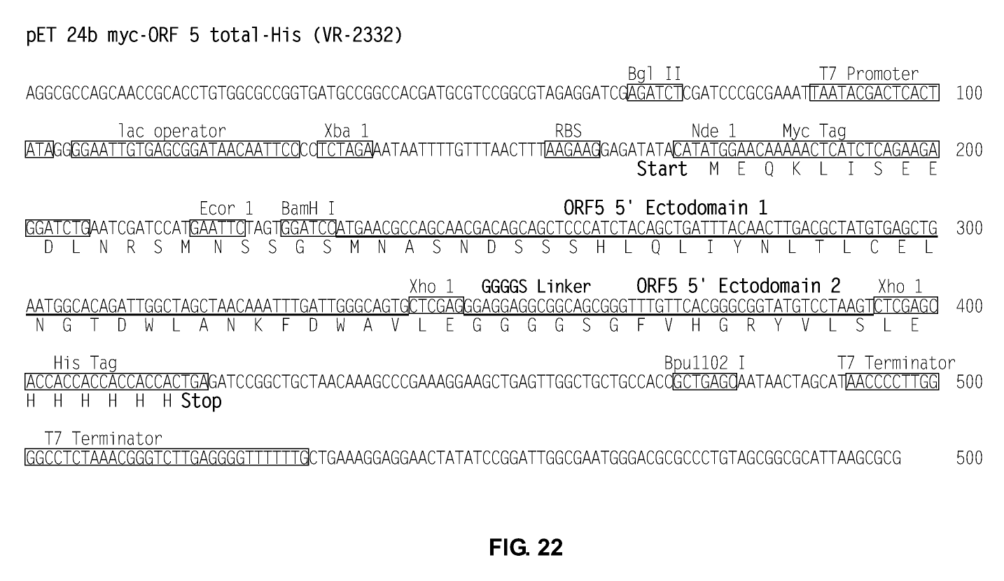

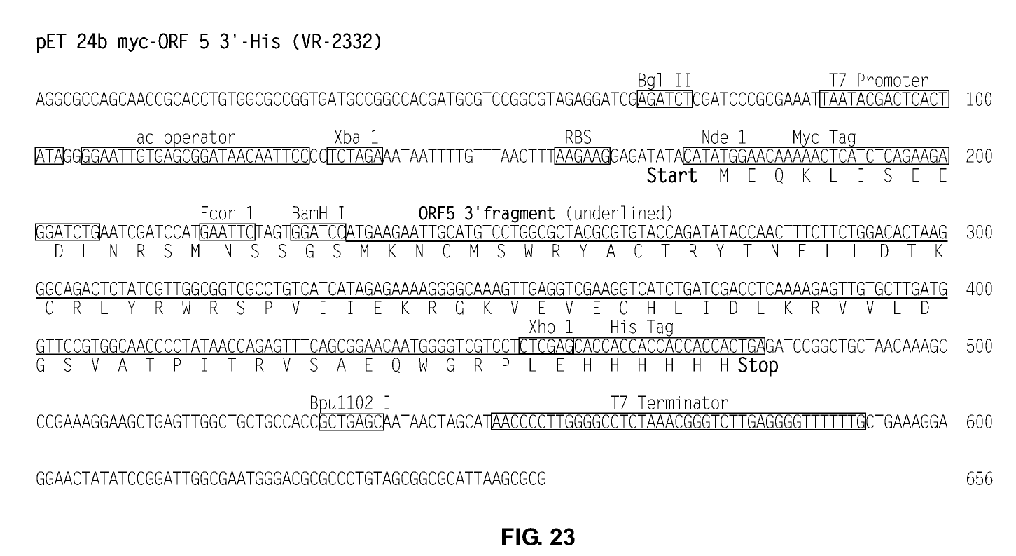

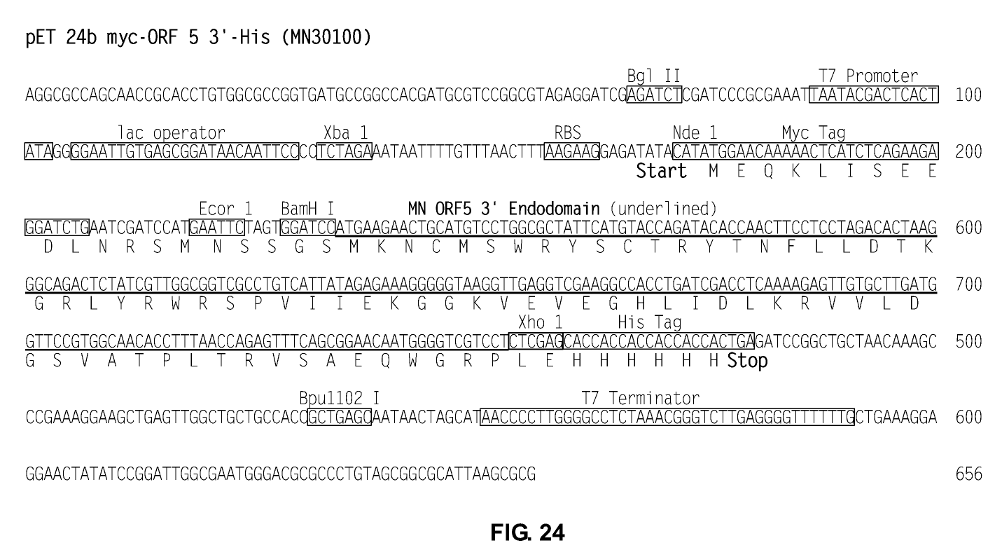

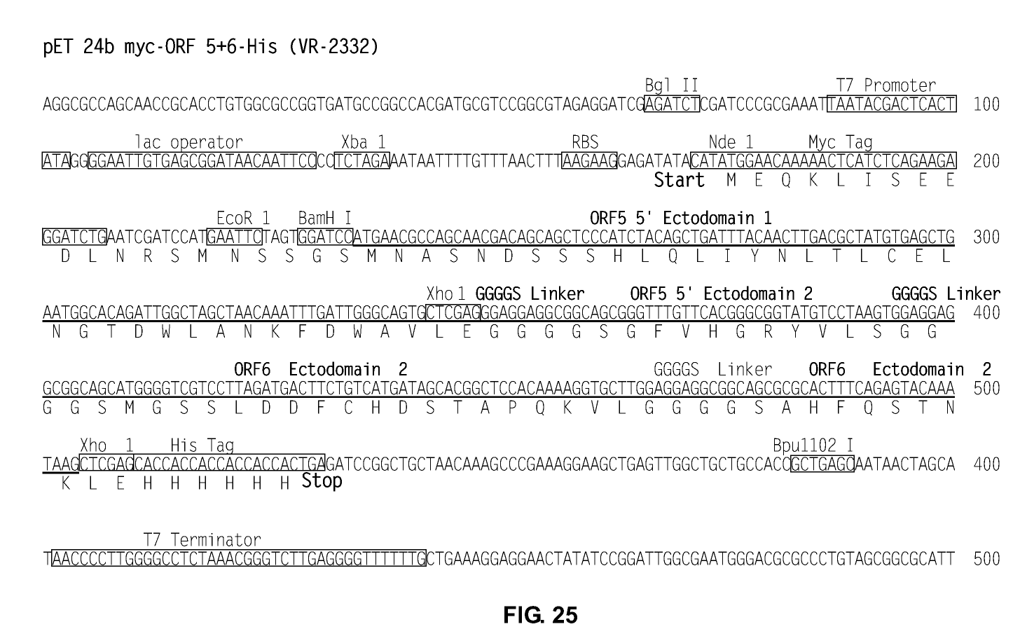

The following polypeptides were produced: a PRRS virus NSP 2P polypeptide (FIG. 19), a PRRS virus first N-terminal ectodomain ORF 5 polypeptide (ORF 5 5′; FIGS. 20 and 21), a PRRS virus first and second N-terminal ectodomains ORF 5 polypeptide (ORF 5′ total; FIG. 22), a PRRS virus endodomain ORF 5 polypeptide (ORF 5 3′; FIGS. 23 and 24), a chimeric polypeptide combining a PRRS virus first and second N-terminal ectodomains ORF 5 polypeptide with a PRRS virus first and second N-terminal ectodomains ORF 6 polypeptide (ORF 5+6; FIG. 25), and a PRRS virus ORF 7 polypeptide. The nucleic acid encoding the polypeptides were from the nucleotide sequences in the VR-2332 strain of PRRS virus (GenBank® Accession No. PRU87392) or the MN30100 strain of PRRS virus.

### PCR Amplification and Cloning

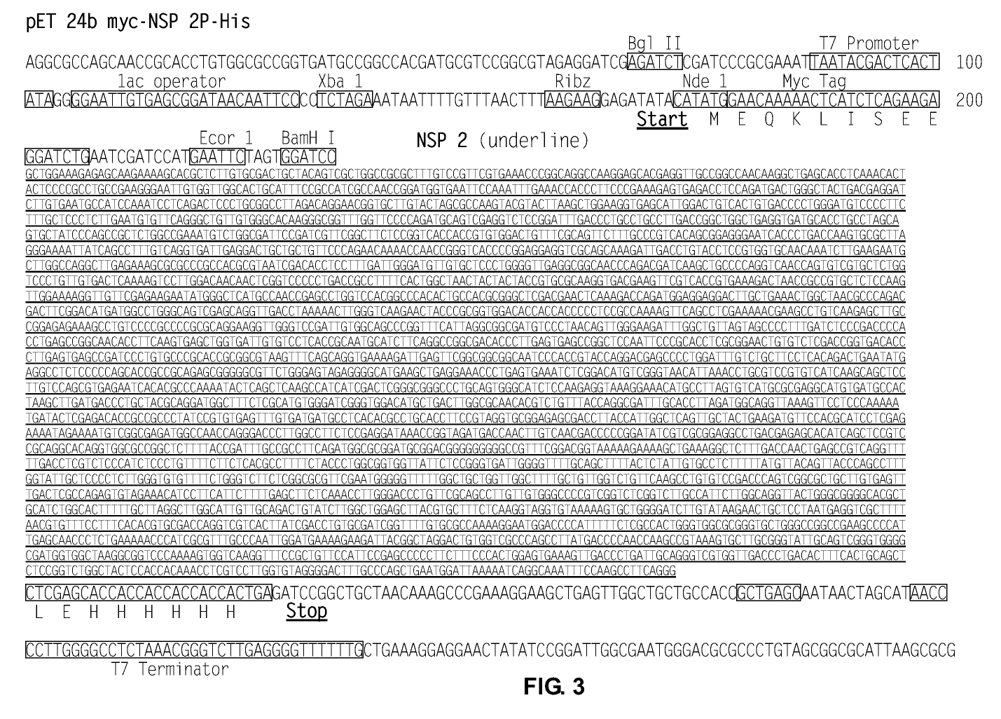

Fragments for cloning were obtained from plasmids prepared as described elsewhere (Nelsen et al., J. Virol., 73:270-280 (1999)). The desired fragments were isolated with appropriate cloning sites by PCR. Primers were designed using Primer3 (Whitehead Institute for Biomedical Research, Cambridge, Mass.). The oligonucleotide primers were obtained from IDT (Coralville, Iowa). The nucleic acid encoding the ORF 7, ORF 5 5′, and ORF 5 3′ polypeptides were PCR amplified in separate reactions using the AmpliTaq Gold® kit (Roche Molecular Systems, Branchburg, N.J.). The nucleic acid encoding the ORF 5 5′ total and ORF 5+6 polypeptide were constructed both by PCR of cDNA and subsequent oligo annealing, PCR amplification, and ligation. The reaction mixtures (50 μL total volume) contained 10× Buffer II (1× concentration), 1.5 mM MgCl₂, 200 μM each of dATP, dCTP, dGTP, and dTTP; 0.2 μM each primer pair; 1.0 U AmpliTaq Gold®, and the appropriate serially diluted (1:10 . . . 1:10,000) mRNA derived cDNA. Upon mixing, the solutions were immediately placed in the thermocycler (GeneAmp PCR system 2400, Perkin Elmer, Shelton, Conn.). Temperature cycle; 1 cycle (95° C. for 10 min); 35 cycles (94° C. for 30 sec, 55° C. for 30 sec, 72° C. for 45 sec); 1 cycle (72° C. for 7 min, 4° C. hold). The resulting amplified DNAs were then separated on an agarose gel. Bands corresponding to the predicted product sizes were gel extracted (Gel Extraction Kit®, Qiagen, Valencia, Calif.) then further purified using the Qiagen PCR Purification Kit® (Qiagen, Valencia, Calif.). The isolated products were then cloned into pGEM T vector (Promega, Madison, Wis.) transformed into DH5α cells (Invitrogen Corp., Carlsbad, Calif.), which were spread on LB 100 mg/mL ampicillin (Amp) agar plates with IPTG and X-Gal. Colonies were color-selected and grown, and the nucleic acid sequenced (Advanced Genetic Analysis Center, University of Minnesota, St, Paul Minn.). After an initial BLAST search screening (GeneBank NCBI, Bethesda, Md.), trace files were edited to remove vector sequence (Seqman® DNASTAR, Inc., Madison, Wis.), and overlapping sequences were aligned (Megalign® DNASTAR, Inc., Madison, Wis.). The nucleic acid encoding the NSP 2P polypeptide was obtained by digesting the clone pET 24b myc-NSP 2-His (FIG. 3) with XhoI and religating the vector.

Sub-Cloning into the Expression Plasmid

Clones were amplified using the appropriate PGEM®T constructs as templates for primers having terminal BamH1 and Xho I sites. The PCR conditions and the insert isolation and purification were standard, followed by restriction digestion (BamHI, XhoI) to prepare the insert for ligation. Ligation conditions were: 100 ng of dephosphorylated vector, 20 ng insert, 1× ligation buffer, and 400 U T4 ligase (New England Biolabs), total volume 10 μL. The ligation reaction was placed at 16° C. for 16 hours before transformation into DH5α cells (Invitrogen Corp., Carlsbad, Calif.). Colonies were selected as previously described and grown, and the nucleic acid sequenced and analyzed (yielding plasmid pET 24b myc-polypeptide-His).

Nucleic acid constructs encoding polypeptides similar to ORF 5 5′ and ORF 5 3′ were also made from MN30100 PRRS virus sequences (Bierk et al., Vet. Rec., 148:687-690 (2001)) starting from cell culture supernatants containing the virus. Viral RNA was obtained from the media by standard procedures. Viral RNA was isolated using the QIAamp viral RNA kit (Qiagen) and stored at −80° C. Purified RNA was converted to cDNA with random hexamers using the GeneAmp RNA PCR kit (Applied Biosystems, Roche Molecular Systems, Branchburg, N.J.). Briefly, 1 μL of a 50 μM solution of random hexamers was combined with 3.0 μg of total RNA to a total volume of 4 μL. The solution was heated at 68° C. for 10 minutes, then quick chilled on ice. 16 μL of a master mix solution was added for a final concentration that contained 10× Buffer II (1× concentration final); 5 mM MgCl₂, 1.0 mM each of dATP, dCTP, dGTP, and dTTP; 20 U/μL Rnase inhibitor, and 25 U/μL reverse transcriptase. This solution was immediately incubated at 25° C. for 10 minutes, 42° C. for 30 minutes, 99° C. for 5 minutes, then 4° C. for 5 minutes. The resulting cDNA was stored at −20° C. and used for PCR amplification.

### Protein Expression

Plasmids pET 24b myc-polypeptide-His were transformed into BL21™ (DE3)-RP cells (Stratagene, River Creek, Tex.), which contain eukaryotic tRNA's for arginine and proline as well as chloramphenicol (cam) resistance. Transformed cells were spread on Kanamycin 30 μg/mL (kan 30), chloramphenicol 35 μg/mL (cam 35) LB plates and screened by colony PCR using the T7, T7 termination primers for the pET 24b plasmid. Ten positive colonies each were grown overnight at 30° C. in 2 mL of 2xYT media (kan 30, cam 35). 200 μL of each of the overnight cultures were used to inoculate ten temperature equilibrated (30° C.) 10 mL aliquots of 2xYT (kan 30). These cultures were grown at 30° C. to an OD_{λ=600} of 0.3; 200 μL was remove for SDS-PAGE analysis; and IPTG was added to a final concentration of 1.0 mM. The induced samples were allowed to grow at 30° C. for 4 hours, then 200 μL was removed for SDS-PAGE analysis. SDS-PAGE gels indicated that all of the colonies expressed polypeptides of the predicted size at high levels (>2 mg/liter media). Expression on a larger scale was done in the same manner scaling up by 100× for a total of 1 liter of media.

### Polypeptide Purification

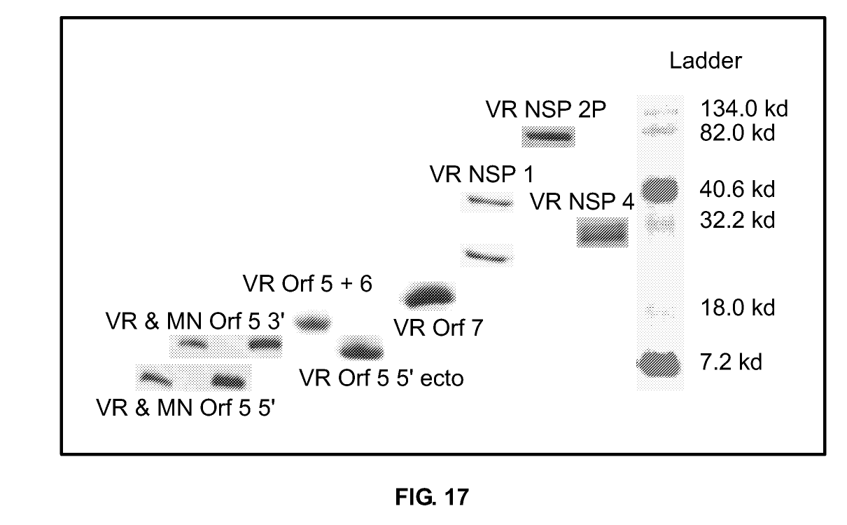

The polypeptides were purified using a modification of the Qiagen Ni-NTA agarose affinity isolation procedure for denatured His tagged polypeptides (Qiagen, Valencia, Calif.). Briefly, 1 liter of induced plasmid containing bacterial cells was pelleted at 4000 g for 20 minutes at 4° C., and the supernatant was decanted off. Immediately following centrifugation, the pellet was resuspended in 30 mL of a solution containing 100 mM NaH₂PO₄, 10 mM Tris-HCl, 8 M Urea, at pH 8.0, and rotated gently at room temperature for 30 minutes. The resulting suspensions were then centrifuged (4° C.) for 30 minutes at 10,000 g to pellet the cellular debris. The supernatant contained high levels of denatured polypeptide and was decanted into 6 mL of 50% Ni-NTA slurry and gently rotated for 1 hour at 4° C. The supernatant-Ni-NTA mixture was then poured into a 1.5 cm ID×30 cm length column and allowed to drain. The column was then washed twice with 20 mL of a solution containing 100 mM NaH₂PO₄, 10 mM Tris-HCl, 8 M Urea, at pH 6.3. The polypeptide was then eluted 4 times with 3 mL aliquots of elution buffer containing 100 mM NaH₂PO₄, 10 mM Tris-HCl, 8 M Urea, at pH 5.9. SDS gel analysis and protein concentration were determined as described above (FIG. 17).

### Polypeptide Refolding

Refolding of the denatured recombinant polypeptides was performed using a variation of the method described elsewhere (Buchner et al., Anal. Biochem., 205(2):263-70 (1992) and Clark, Curr. Opin. Biotechnol., 9(2):157-63 (1998)). First, denatured polypeptide solutions containing pure polypeptide were pooled and dialyzed (Spectra/Por MWCO® 6-8,000, Spectrum Laboratories, Rancho Dominguez, Calif.) for 4 hours at 4° C. against 500 mL of 0.1 M Tris, pH 8.0, 6 M guanidine-HCl, and 2 mM EDTA. The dialysis was then repeated with fresh buffer for an additional 4 hours. After adjusting the polypeptide concentration to 3 mg/mL (concentration determination using the Bio-Rad RC DC protein assay kit, Bio-Rad, Hercules, Calif.), dithiothreitol (DTT) was added yielding a final concentration of 300 mM DTT. This solution (5 mL) was allowed to stir at room temperature for 2 hours followed by filtration using a 0.45 μm filter (Syringe Filter, Fisher Scientific, Pittsburgh, Pa.). The reduced polypeptide solution was then added rapidly at 4° C. with moderate stirring into 500 mL of refolding buffer (100 mM Tris at pH 8.0, 0.5 M L-Arginine, 8 mM oxidized glutathione, 2 mM EDTA, 10 μM pepstatin A, 10 μM leupeptin, 1 mM PMSF) corresponding to a final dilution of about 1:100. The resulting solution was then filtered through a 0.22 μm membrane (Steritop, Millipore, Bedford Mass.) to remove particulates and left to stir overnight. The purified polypeptide was concentrated by tangential flow filtration cassette (Pellicon XL Ultracel PLC 5 kd, Millipore, Bedford Mass.) to a volume of 10 mL followed by dialysis (Spectra/Por MWCO® 6-8,000, Spectrum Laboratories, Rancho Dominguez, Calif.) against 20 mM Tris HCl, at pH 8.0. Polypeptide concentrations were determined using the Bio-Rad RC DC protein assay kit (Bio-Rad, Hercules, Calif.), quantitative SDS gel, and the Agilent 2100 bioanalyzer (Protein LabChip® Kit, Agilent Technologies, Palo Alto, Calif.). Purified polypeptide solutions were stored at −80° C. The polypeptides ORF 5 5′, ORF 5 3′, and ORF 5 total were not routinely refolded because refolding did not affect ELISA reactivity.

### ELISA

Polypeptides were diluted in carbonate buffer (15 mM Na₂CO₃, 35 mM NaHCO₃, pH 9.6) to a concentration of 1 μg/mL. Each of the ELISA plate wells (COSTAR 3590, 96 Well EIA/RIA plate, Corning Inc., Corning, N.Y.) was then coated with 100 μL of the appropriate polypeptide carbonate solution (providing 100 ng of polypeptide per well), then incubated at 4° C. overnight. Samples were run in duplicate. A set of wells was left uncoated for determination of serum and secondary antibody background effects (found to be less that 0.005 Absorbance units). The plates were then washed (EL-404 Microplate Washer, Bio-Tek Instruments Inc. Winooski, Vt.) six times with PBS-Tween-20 (0.1%) at room temperature. Non-specific binding sites were blocked with 300 μL/well of PBS-Tween (0.1%) containing 3% nonfat dried milk (NFDM) for 2 hour at room temperature. The plates were then washed as described above. Serum samples were then diluted 1:2000 with PBS-Tween-20 (0.1%) containing 3% NFDM, then 100 μL was added to the appropriate wells, and the plates equilibrated at room temperature for 2 hours. The titer values using serial dilution indicated that 1:2000 serum dilutions demonstrated similar data trends (within error). The wells were then washed as before. Secondary detection antibody (peroxidase labeled goat anti-swine IgG (H+L), Kirkegaard & Perry Laboratories Inc. (KPL) Gaithersburg, Md.) was diluted 1:5000 in PBS-Tween (0.1%) containing 3% NFDM, 100 μL of the diluted solution was added to each well. After incubating for 1 hour at room temperature, the plates were again washed. Tetra methyl benzidine (TMB cat #50-76-00 Kirkegaard & Perry Laboratories Inc. (KPL) Gaithersburg, Md.) was used to perform the calorimetric analysis. Equal volumes of TMB peroxidase (solution A) and peroxidase (solution B) were mixed together, and 100 μL was added to each well. The solution was allowed to develop for 15 minutes at room temperature (blue color). The reactions were then quenched by adding 100 μL of 1 M phosphoric acid (yellow color). Plates were read at 450 nm (Thermo Max microplate reader, Molecular Devices, Sunnyvale, Calif.).

### Example 3

### PRRS Virus Antibody Responses Following Repeated Homologous Wild-Type Virus Challenges

Serology has been the cornerstone of veterinary disease monitoring and control. The presence of specific antibodies in serum indicates prior exposure to disease, and may also confirm that the animal possesses protective immunity. Currently available PRRS virus ELISA antibody tests may not be sensitive for all possible situations found in infected groups of pigs, particularly re-infected animals. Many animals return to seronegative status within 4 to 6 months after initial infection (Yoon et al., J. Vet. Diagn. Invest., 7:305-312 (1995)). In addition, there have been reports of animals returning to and remaining ELISA antibody negative during multiple repeated vaccinations with a modified live PRRS virus vaccine (Baker et al., Proc. Allen D. Leman Swine Conference, vol. 26 (suppl.) p. 31 (1999)). If loss of ELISA antibody response were to occur after repeated frequent exposures to the same wild-type PRRS virus, it might alter the way veterinarians interpret PRRS virus ELISA test results for their clients when monitoring herds for continued virus circulation.

The following experiment was performed to (1) determine whether PRRS virus ELISA seronegative animals can be induced by multiple low-dose immunizations with wild-type virus and (2) characterize the expression timeline for PRRS virus serum neutralizing antibodies and antibodies to individual recombinant ORF polypeptides.

Sixty-eight PRRS virus-negative 6 month old barrows were injected twice, one month apart, and then every other month approximating a 6/60 type schedule for a total of 6 immunizations using 10^{2.5} field strain SD 28983 PRRS viruses per dose. The animals were bled 3 weeks following each immunization, and the samples tested for PRRS virus ELISA and serum neutralizing antibodies. Four months after the last immunization (12 months after initial exposure), the animals were challenged again with SD 28983.

The blood samples were tested for serum neutralization antibodies by fluorescent focus neutralization (strain 23983 virus as assay inoculum) and for antibodies to recombinant PRRS virus polypeptides obtained as described herein. Briefly, PRRS virus rORF polypeptides were produced by inserting the desired cDNA nucleic acid fragments into E. coli for expression. The polypeptides produced included nucleocapsid (an ORF 7 polypeptide) and a chimera polypeptide fragment that contained the ectodomain regions of both an ORF 5 envelope polypeptide and an ORF 6 matrix polypeptide, which co-localize within the viral envelope. ELISA plates were coated with each polypeptide and serum samples were tested by limiting dilution. Results were recorded as titers rather than optical density ratios. The blood samples also were tested using a commercially available PRRS virus ELISA (2XR PRRS virus antibody test kit; IDEXX Laboratories).

The PRRS virus 2XR ELISA antibody levels dropped sharply after initial sero-conversion, even in the face of repeated injections with virulent 28983 strain PRRS virus. Nearly all animals developed solidly positive antibody responses initially. 75 percent of these animals, however, returned to sero-negative status 4 months after the 6th injection with live virus. This is similar to that observed in sows following multiple vaccinations with MLV PRRS virus vaccine (Baker et al., Proc. Allen D. Leman Swine Conference, vol. 26 (suppl.) p. 31 (1999)). Conversely, the serum neutralization test detected antibody later following initial infection, and all animals remained serum neutralization antibody positive at the end of the experiment.

The rORF ELISAs revealed temporal antibody curves. The assay using recombinant nucleocapsid polypeptides resulted in a curve that followed the IDEXX 2XR ELISA response curve closely, falling to low levels at 4 months. Conversely, the envelope chimera ORF 5 and ORF 6 polypeptide ELISA followed a temporal pattern nearly identical to the PRRS serum neutralization antibody response curve. Thus, it appears that pigs initially produce strong antibody responses directed predominantly against nucleocapsid polypeptides, but over time the antibody response is redirected to the envelope polypeptides. It appears that the immune response to PRRS virus is slow to shift to immunologically protective serum neutralization antibodies.

These results demonstrate that an effective diagnostic kit can include ORF 5 polypeptides and ORF 6 polypeptides. These results also demonstrate that a weak IDEXX PRRS virus ELISA antibody response following vaccination or re-exposure may paradoxically indicate that the animal has a protective immune response against that vaccine or virus, since the IDEXX PRRS virus ELISA kit appears to be limited to detecting antibodies that bind PRRS virus nucleocapsid polypeptides.

### Example 4

### Comparative Antibody Responses to Virulent and Attenuated Strains of PRRS Virus

The following experiment was performed to (1) characterize the antibody response of pigs to individual PRRS virus polypeptides, (2) determine the antibody responses to viral isolates that vary in virulence, and (3) determine the relationship between antibody response and protection to challenge.

One hundred PRRS-negative 3-4 week-old piglets were divided into groups. Ten pigs per group were inoculated intranasally with 2×10³ TCID₅₀ PRRS virus strains characterized as highly or moderately virulent (SDSU73, MN 184, JA 142, and 17198-6), low virulent (VR2332 and ABST-1), or avirulent (Ingelvac PRRS and Ingelvac ATP). One group of ten pigs received a cocktail containing equal amounts of all viruses, and ten control pigs received no virus. After animals were fully recovered from acute infection, they were challenged with MN 184. Clinical signs were recorded throughout the experiment and necropsies were performed 14 days after challenge. Animals were bled weekly, and antibody levels were determined by ELISA to purified nonstructural and structural polypeptides that were produced by inserting the desired cDNA fragments into E. coli for expression and purification. The polypeptides included a VR2332 PRRS virus ORF 7 polypeptide (a nucleocapsid polypeptide), a VR2332 PRRS virus NSP 1 polypeptide, a VR2332 PRRS virus NSP 2P polypeptide, a VR2332 PRRS virus NSP 4 polypeptide, a VR2332 ORF 5 5′ ectodomain 1 polypeptide, an MN30100 VR2332 ORF 5 5′ ectodomain 1 polypeptide, a VR2332 ORF 5 3′ endodomain polypeptide, an MN301000RF 5 3′ endodomain polypeptide, a VR2332 ORF 5 5′ ectodomains 1 and 2 polypeptide, and a VR2332 ORF 5/ORF 6 chimeric polypeptide (ORF 5 5′ ectodomains 1 and 2 plus ORF 6 5′ ectodomains 1 and 2; also referred to as a GP5-M chimeric ectodomain polypeptide). ELISA plates were coated with each polypeptide, and the serum samples were tested at a dilution of 1/2000. Specific antibody levels were expressed as background-corrected optical density values.

Clinical responses to PRRS virus inoculation ranged from no or minimal observed effects in animals given avirulent or lowly virulent strains, to death in about 50 percent of animals administered MN 184. Antibody responses to animals inoculated with highly and moderately virulent strains were pronounced. Antibodies usually first appeared at 21 days and peaked at 28 days after infection. The level of antibodies to nucleocapsid declined dramatically after day 28, whereas the response to other viral polypeptides tended to be maintained at high levels to the end of the experiment. Antibody responses to nonstructural polypeptides NSP 1 and NSP 2 were as high or higher than the response to nucleocapsid, but the response to NSP 4, encoding a viral protease, was low at all time points.

Although the humoral response to viral administration was IDEXX-positive in all treatment groups, marked variations in the intensity of antibody responses were apparent. Avirulent and lowly virulent strains elicited less robust antibody responses as compared to moderate or highly virulent strains. These differences were present across all antigens tested. However, response to challenge was similar among all treatment groups.

In summary, differences in antibody response to various structural and nonstructural PRRS virus polypeptides were observed. In addition, variation in antibody responses to virulent strains of PRRS virus as compared to their attenuated forms were observed. The differences in antibody responses, however, were not associated with protection against re-infection with a heterologous, highly virulent challenge strain. These findings are the first characterization of antibody responses to individual PRRS virus polypeptides throughout acute infection and following virulent challenge.

### Example 5

### Detecting Antibodies to PRRS Virus Using Individual PRRS Virus Polypeptides Verses a Commercially Available ELISA Kit

The following experiment was performed to determine whether particular polypeptide ELISAs can detect PRRS virus positive samples under conditions of multiple exposure and extended time periods in which the IDEXX ELISA changes from positive to negative. PRRS-negative 6 month old barrows were injected with 10^{2.5} tissue culture infective dose 50% (TCID₅₀) of field strain SD 28983 PRRS virus initially, then at one, two, four, six, and eight months for a total of 6 inoculations. Animals were bled preceding each inoculation, and serum was collected. The blood samples were tested using (1) a commercially available PRRS virus ELISA (2XR PRRS virus antibody test kit; IDEXX Laboratories) or (2) an ELISA containing particular PRRS virus polypeptides. The IDEXX 2XR HerdChek® ELISA was performed according to the manufacturer's directions on serum samples diluted 1/40. Data are presented as means and standard deviation of all samples in each group. Group size varied from 19-23 samples from 23 pigs per group.

About half the animals analyzed using the IDEXX kit were found to be negative at the 353 day time point (Table 3 and FIG. 10). An S/P ratio greater than 0.4 indicated that the sample was positive for antibodies to PRRS virus, while an S/P ratio less than 0.4 indicated that the sample was negative for antibodies to PRRS virus.

TABLE 3
Analysis of samples using the IDEXX kit.

| | | Number | Number |
|---|---|---|---|
| | Average | Samples | Samples |
| Days | S/P | Positive | Negative |
| 0 | 0.124 | 0 | 22 |
| 29 | 1.273 | 22 | 1 |
| 59 | 1.033 | 20 | 1 |
| 132 | 0.697 | 17 | 2 |
| 270 | 0.629 | 18 | 4 |
| 353 | 0.480 | 11 | 11 |

The same samples were analyzed using either recombinant GP5 endodomain polypeptides or recombinant GP5-M chimeric polypeptides in ELISAs. Briefly, plates were coated with 100 ng polypeptide per well in carbonate pH 9.6 overnight. Sera were diluted 1/1000 and tested in duplicate. For the GP5 endodomain polypeptide ELISAs, data are presented as the sample/positive ratio of unadjusted OD values of all samples in each group and the number of samples with a mean greater than (Positive) or less than (Negative) 0.21. For the GP5-M chimeric polypeptide ELISAs, data are presented as the sample/positive ratio of unadjusted OD values of all samples in each group and the number of samples with a mean greater than (Positive) or less than (Negative) 0.5. The sample/positive ratio was determined as the sample OD minus OD of control wells without antigen/positive control OD minus OD of control wells without antigen.

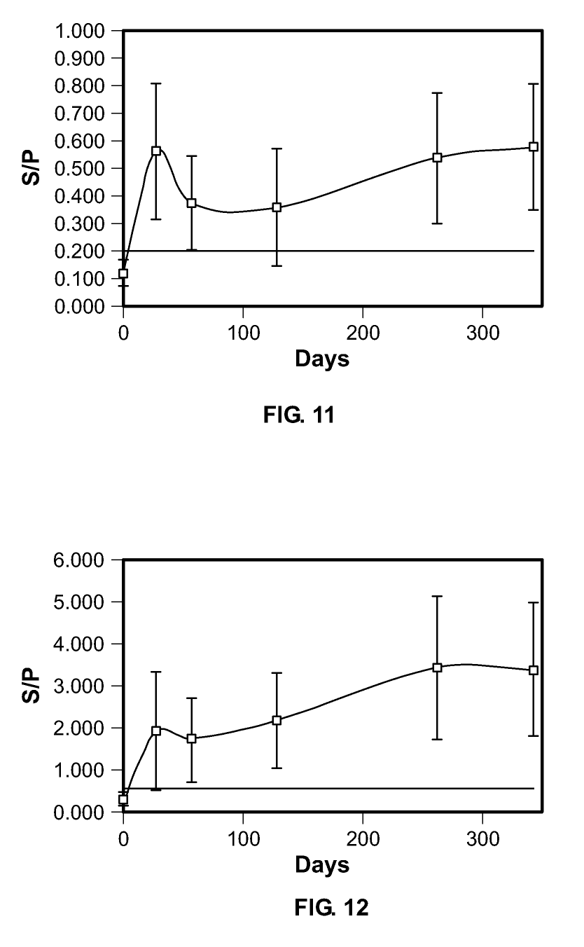

Two samples were found to be negative for antibodies to the tested PRRS virus GP5 endodomain polypeptide at the 353-day time point (Table 4 and FIG. 11). When the GP5-M chimeric polypeptide was used, all the tested samples were found to be positive at the 353-day time point (Table 5 and FIG. 12). These results demonstrate that assays using GP5 polypeptides can detect anti-PRRS virus antibodies in situations where the commercially available IDEXX kit can not.

TABLE 4
Analysis of samples using a GP5 endodomain polypeptide in an ELISA.

| | | | Number | Number |
|---|---|---|---|---|
| | | Average | Samples | Samples |
| Days | Months | S/P | Positive | Negative |
| 0 | 0.000 | 0.123 | 0 | 22 |
| 29 | 1.000 | 0.565 | 23 | 0 |
| 59 | 2.000 | 0.374 | 19 | 2 |
| 132 | 4.400 | 0.360 | 15 | 4 |
| 270 | 9.000 | 0.537 | 23 | 0 |
| 353 | 11.800 | 0.576 | 20 | 2 |

TABLE 5
Analysis of samples using a GP5-M chimeric polypeptide in an ELISA.

| | | | Number | Number |
|---|---|---|---|---|
| | | Average | Samples | Samples |
| Days | Months | S/P | Positive | Negative |
| 0 | 0.000 | 0.333 | 0 | 22 |
| 29 | 1.000 | 1.929 | 21 | 2 |
| 59 | 2.000 | 1.722 | 20 | 1 |
| 132 | 4.400 | 2.157 | 19 | 0 |
| 270 | 9.000 | 3.378 | 23 | 0 |
| 353 | 11.800 | 3.318 | 22 | 0 |

### Example 6

### Detecting Antibodies to PRRS Virus Using a Mixture of PRRS Virus Polypeptides Verses a Commercially Available ELISA Kit

Ten weaned pigs per group were each inoculated intranasally with 2 mL of tissue culture media containing 3.0 Log₁₀ TCID₅₀/mL of the virus isolates listed in Table 6. The pool was a mixture of equal portions of each of the eight isolates. The control was tissue culture media alone.

TABLE 6
PRRS virus isolates.

| | Group | |
|---|---|---|
| Isolate | number | Virulence |
| VR 2332 | 1 | Moderate |
| Ingelvac ® PRRS MLV* | 2 | Attenuated VR2332 |
| JA 142 | 3 | High |
| Ingelvac ® PRRS ATP* | 4 | Attenuated JA 142 |
| SDSU 73 | 5 | High |
| Abst-1* | 6 | Attenuated SDSU 73 |
| MN 184 | 7 | High |
| 17198 | 8 | High |
| Pool** | 9 | High |
| Control | 10 | N/A |

*Attenuated PRRS virus isolates.
**Mixture containing all of the eight isolates.

To compare the IDEXX ELISA kit with a recombinant polypeptide ELISA, sera were selected blindly from five pigs per group at day 7 after inoculation. Four of the five samples were also tested on day 14. For the IDEXX ELISA kit, an S/P ratio ≧0.4 indicated that the sample was positive, while an S/P ratio <0.4 indicated that the sample was negative. The same samples were analyzed using the IDEXX ELISA kit and the recombinant polypeptide ELISA.

For the recombinant polypeptide ELISA, the following polypeptides were expressed and purified as described herein: an ORF 7 polypeptide, a ORF 5+6 chimeric ectodomains polypeptide, an NSP 2P polypeptide, and an ORF 5 3′ endodomain polypeptide. The purified polypeptide concentrations were determined by agreement among OD₂₈₀ absorbance, Agilent bioanalyzer analysis, SDS PAGE, and RC DC Lowry protein assay. Microtiter plates were coated with 150 ng of each polypeptide for a total of 600 ng in 100 μL carbonate, pH 9.6, coating buffer. ELISAs were performed on serum samples at a 1/500 dilution. The day 7 and day 14 samples are the identical samples as were analyzed by IDEXX ELISA. In addition, 7 animals per group (5 in the MN184 and pool groups) were analyzed at day 50. Control animals were negative at all times. Seven pigs in group 10 were tested and were negative at all tested days.

Only one of the tested samples collected on day 7 was positive when analyzed using the IDEXX kit (Table 7). In addition, thirteen samples collected on day 14 were negative for PRRS virus antibodies. Twenty-three of the 36 samples collected on day 14 were positive for PRRS virus antibodies.

TABLE 7
Results with IDEXX ELISA kit.

| | | Number | Number |
|---|---|---|---|
| | Average | Samples | Samples |
| Day | S/P | Positive | Negative |
| 7 | 0.116374 | 1 | 44 |
| 14 | 0.753558 | 23 | 13 |

In contrast, 14 of the tested 45 samples collected on day 7 were positive when analyzed using the ELISA containing the mixture of PRRS virus polypeptides (Table 8). In addition, only eight of the 36 samples collected on day 14 were negative for PRRS virus antibodies. Twenty-eight of the 36 samples collected on day 14 were positive for PRRS virus antibodies. Further, 54 of the 59 samples collected on day 50 were positive for PRRS virus antibodies (Table 8).

TABLE 8
Results with ELISA containing the mixture of PRRS virus polypeptides.

| | | Number | Number | |
|---|---|---|---|---|
| | Average | Samples | Samples | Cutoff |
| Days | S/P | Positive | Negative | value |
| 7 | 0.558178 | 14 | 31 | 0.4 |
| 14 | 1.549222 | 28 | 8 | 0.4 |
| 50 | 3.024511 | 54 | 5 | 0.55 |

These results demonstrate that mixtures of PRRS virus polypeptides can be used to detect PRRS virus antibodies in animals exposed to PRRS virus at time points that are not only early but also late with respect to the time of PRRS virus exposure. For example, positive samples were detected at day 7, 14, and 50 following PRRS virus exposure.

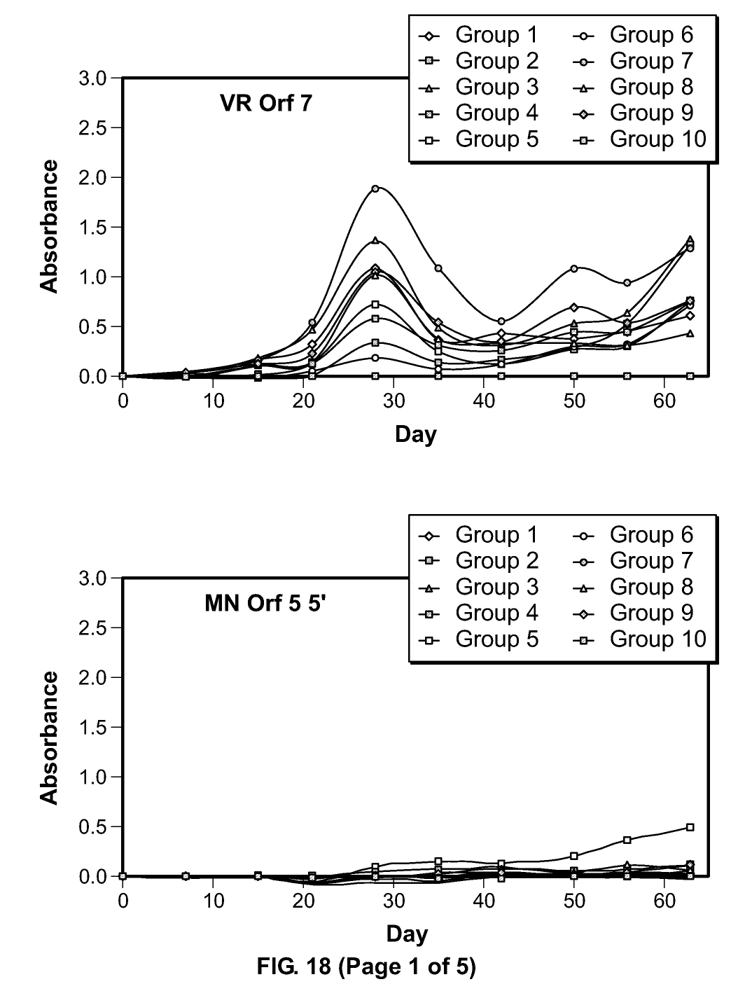

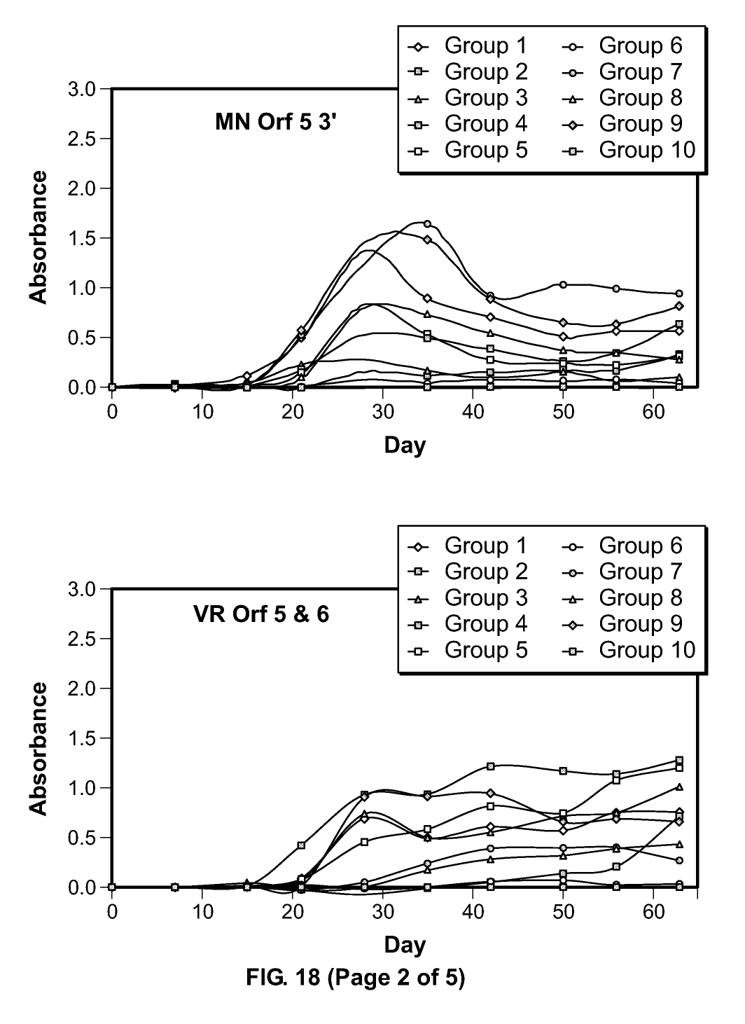

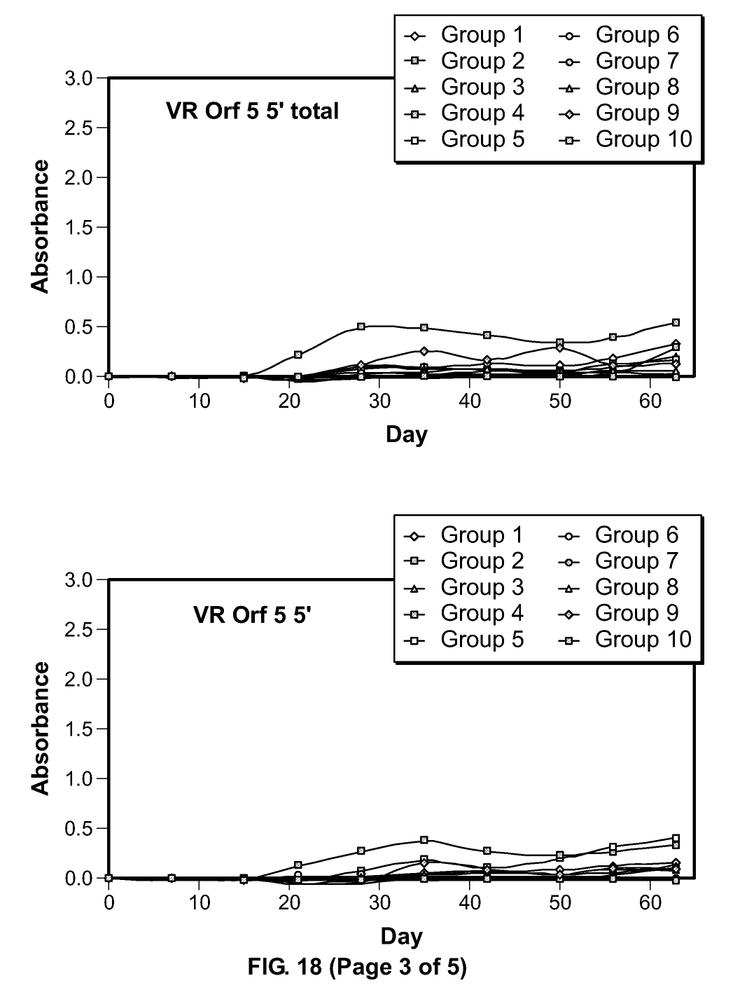

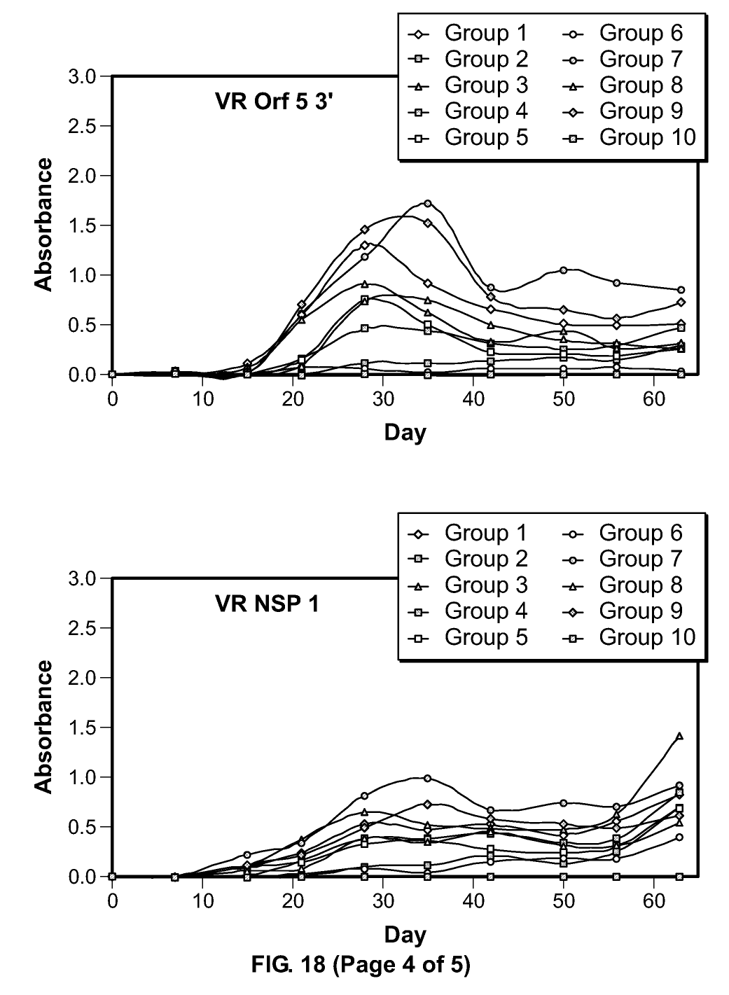

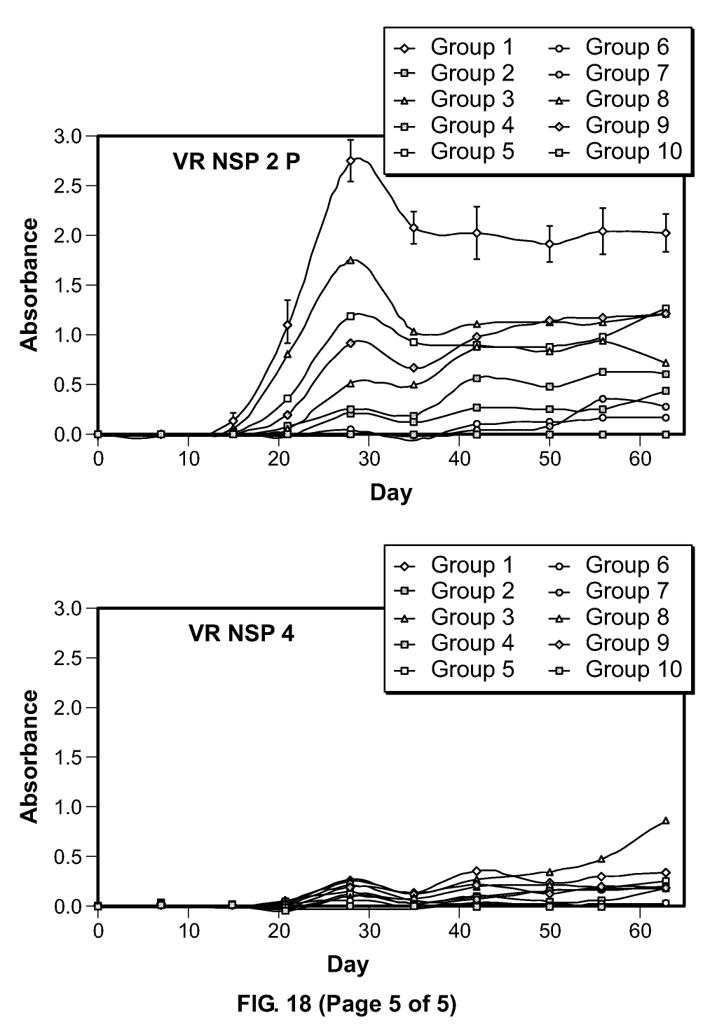

The samples for each group were also tested using ELISAs with an individual PRRS virus polypeptide obtained as described herein (FIG. 18).

### Example 7

### Differentiating Between Animals Exposed to Vaccine or Field Strains of PRRS Virus

Twenty-eight days after vaccination with the Ingelvac MLV (also referred to as RespPRRS; GenBank® Accession Number AF066183), serum samples were obtained from 5 pigs, diluted 1/300, and analyzed in duplicate on ELISA plates coated with 200 ng of a 5′ ectodomain ORF 5 polypeptide from PRRS virus strain VR2332, a 5′ ectodomain ORF 5 polypeptide from PRRS virus isolate MN30100, a 3′ endodomain ORF 5 polypeptide from PRRS virus strain VR2332, or a 3′ endodomain ORF 5 polypeptide from PRRS virus isolate MN30100. Twenty-eight days after inoculation with PRRS virus isolate MN30100, serum samples were obtained from 5 pigs and analyzed in the same manner. The 5′ ectodomain of PRRS virus ORF 5 polypeptides contains an amino acid sequence that is variable among PRRS virus isolates, while the 3′ endodomain of PRRS virus ORF 5 polypeptides contains an amino acid sequence that is conserved among PRRS virus isolates.

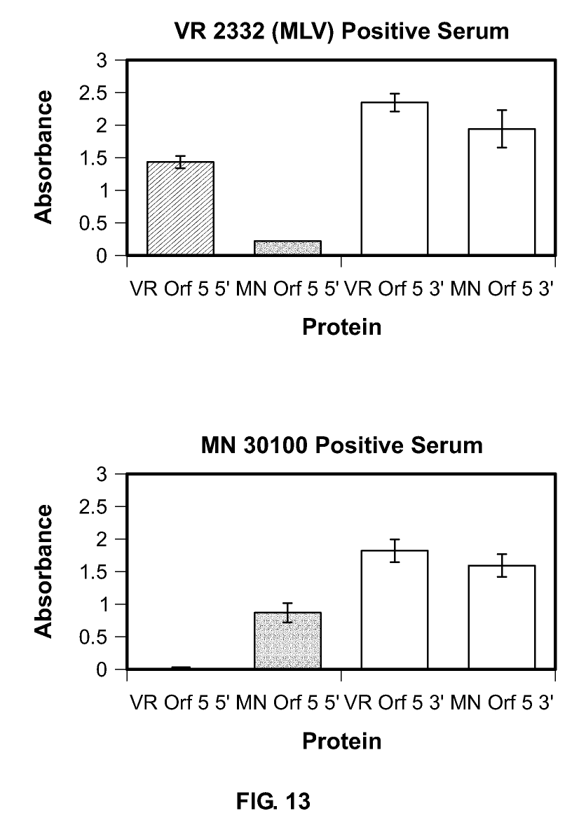

The ELISAs containing the 3′ endodomain ORF 5 polypeptides (either the 3′ endodomain ORF 5 polypeptide from VR2332 or the 3′ endodomain ORF 5 polypeptide from MN30100) detected PRRS virus antibodies in samples obtained from animals exposed to either the vaccine strain (Ingelvac MLV) or the field isolate (MN30100) of PRRS virus (Table 9 and FIG. 13). These results demonstrate that a polypeptide limited to a PRRS virus sequence that is conserved among PRRS viruses, whether from a vaccine version or a wild-type version of PRRS virus, can be used to detected animals exposed to any type of PRRS virus (e.g., a vaccine version or wild-type version of PRRS virus).

TABLE 9
ELISA results of serum samples obtained from pigs exposed to PRRS
virus Ingelvac MLV or MN30100 and reacted with polypeptide fragments
from either PRRS virus Ingelvac MLV or MN30100.

| Polypeptide for | Virus animal exposed | | Standard | |
|---|---|---|---|---|
| ELISA: | to: | Average* | Deviation | SEM |
| 5′ ORF 5 | MN30100 | 0.009 | 0.05303 | 0.037 |
| (VR2332) | | | | |
| | MLV | 1.443 | 0.11172 | 0.079 |
| 5′ ORF 5 | MN30100 | 0.946 | 0.22273 | 0.157 |
| (MN30100) | | | | |
| | MLV | 0.193 | 0.00636 | 0.004 |
| 3′ ORF 5 | MN30100 | 1.985 | 0.26269 | 0.185 |
| (VR2332) | | | | |
| | MLV | 2.336 | 0.19940 | 0.141 |
| 3′ ORF 5 | MN30100 | 1.726 | 0.27930 | 0.197 |
| (MN30100) | | | | |
| | MLV | 1.932 | 0.42426 | 0.300 |

*The data are specific OD values after subtraction of background.

The ELISAs containing the 5′ ectodomain ORF 5 polypeptide from VR2332 detected PRRS virus antibodies in samples obtained from animals exposed to the vaccine strain (Ingelvac MLV) and did not detect PRRS virus antibodies in samples obtained from animals exposed to the field isolate (MN30100) of PRRS virus (Table 9 and FIG. 13). Likewise, the ELISAs containing the 5′ ectodomain ORF 5 polypeptide from MN30100 detected PRRS virus antibodies in samples obtained from animals exposed to the field isolate (MN30100) of PRRS virus and did not detect PRRS virus antibodies in samples obtained from animals exposed to the vaccine strain (Ingelvac MLV)(Table 9). These results demonstrate that a polypeptide limited to a PRRS virus sequence from a vaccine version of a PRRS virus that is variable among PRRS viruses can be used to identify animals exposed to the vaccine version of PRRS virus as opposed to animals exposed to a wild-type version of PRRS virus.

### Example 8

### Producing Additional Recombinant PRRS Virus Polypeptides from Vaccine Strains and Field Isolates

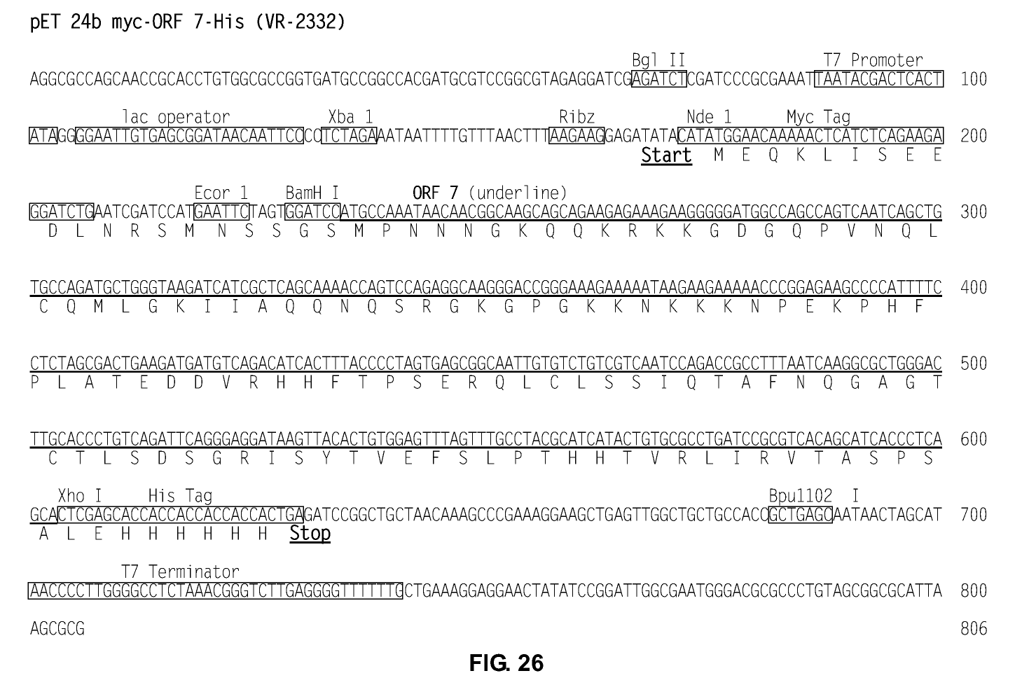

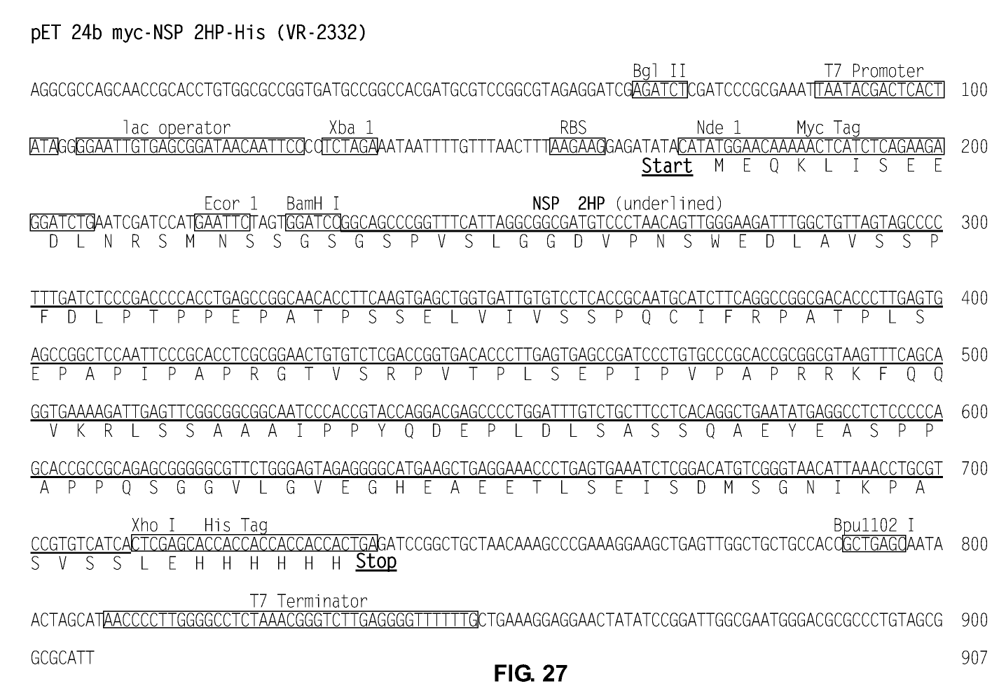

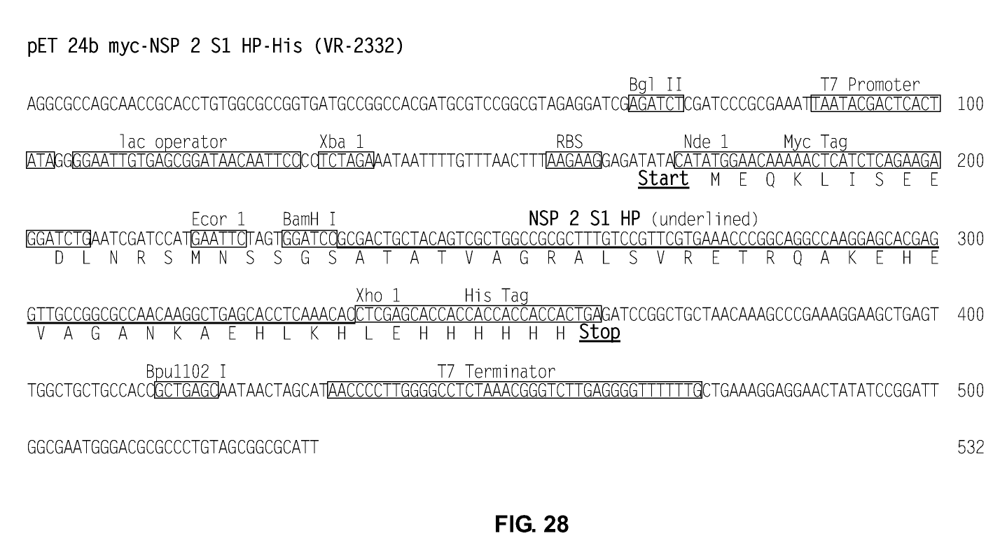

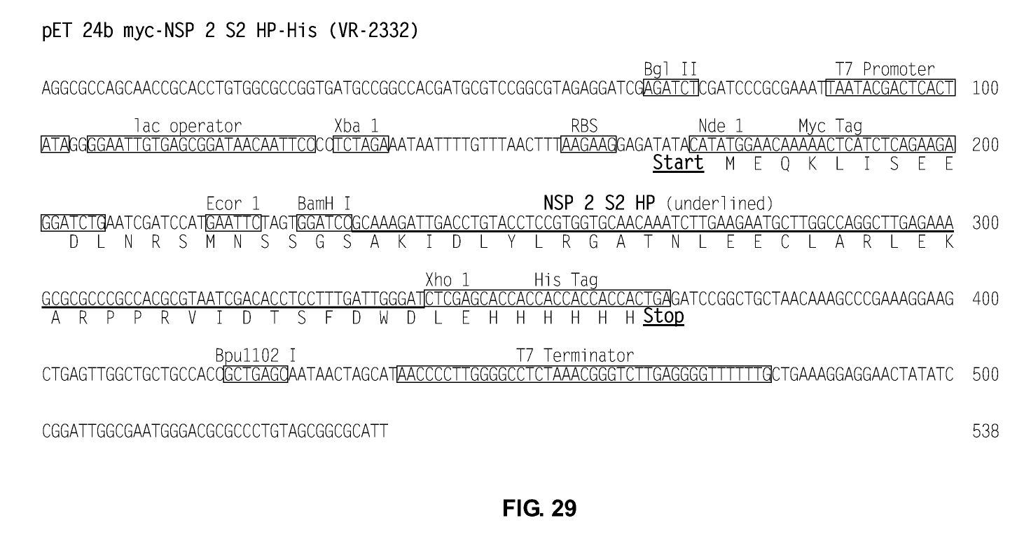

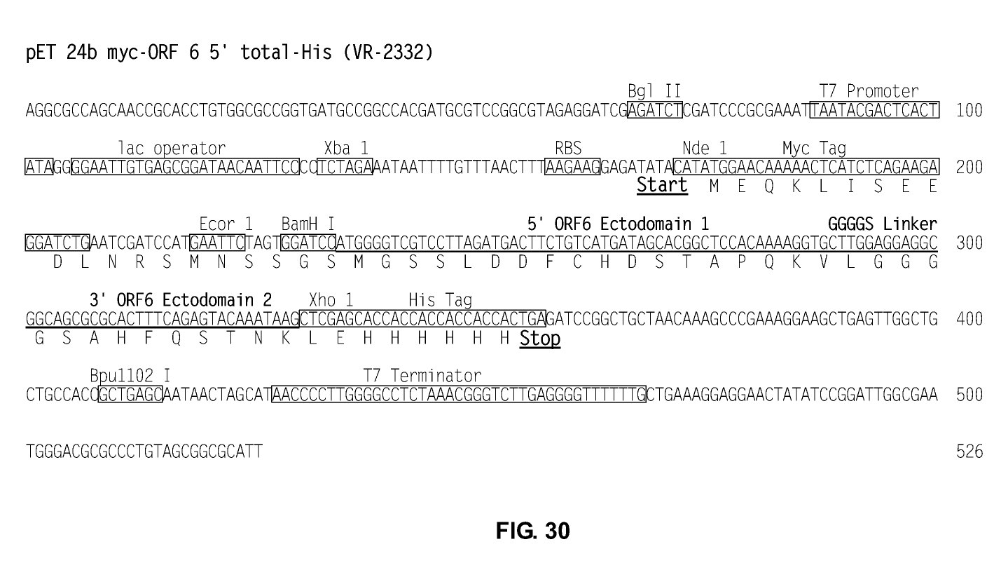

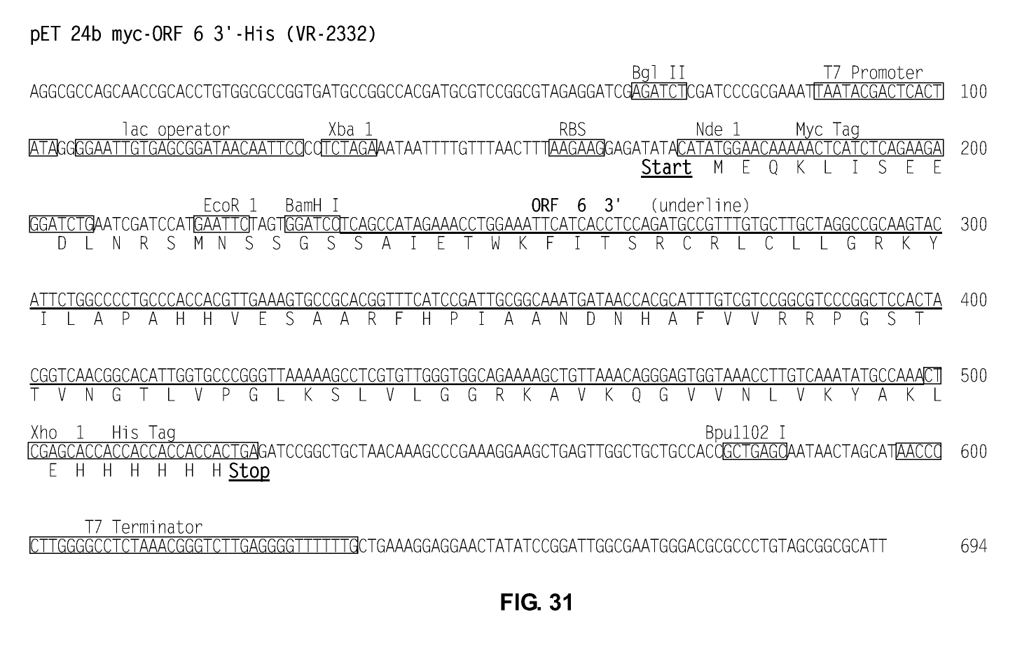

The following polypeptides were produced: a PRRS virus ORF 7 polypeptide (FIG. 26), a PRRS virus NSP 2HP polypeptide (FIG. 27), a PRRS virus NSP 2 S1 HP polypeptide (FIG. 28), a PRRS virus NSP 2 S2 HP polypeptide (FIG. 29), a PRRS virus first and second N-terminal ectodomains ORF 6 polypeptide (ORF 6 5′ total; FIG. 30), and a PRRS virus endodomain ORF 6 polypeptide (ORF 6 3′; FIG. 31). In each case, the polypeptides contained a myc sequence followed by the PRRS virus sequence followed by a polyhistidine sequence. The nucleic acid encoding the PRRS virus sequence of these polypeptides was from the nucleotide sequence in the VR-2332 strain of PRRS virus (GenBank® Accession No. PRU87392). In addition, a myc-ORF 7-His polypeptide, a myc-ORF 5 3′-His polypeptide, a myc-ORF 6 3′-His polypeptide, and a myc-NSP 2-His polypeptide was produced. The nucleic acid encoding the PRRS virus sequence of these polypeptides were from the nucleotide sequence in the Lelystad Virus (LV) strain of PRRS virus (GenBank® Accession No. M96292). A myc-NSP 2 HP-His polypeptide also was produced. The nucleic acid encoding the PRRS virus sequence of this polypeptide was from the nucleotide sequence in the Boehringer Ingelheim vaccine strain ATP. GenBank® Accession No. AY424271 for PRRS virus strain JA142 was used to obtain the ATP sequences since PRRS virus strain JA142 is the parental virus strain of the Boehringer Ingelheim vaccine strain ATP.

The polypeptides containing a PRRS sequence of VR-2332 were constructed as described in Example 2. For plasmids containing sequences of PRRS virus strains LV or ATP, viruses were isolated from cell culture lysates by centrifugation through a sucrose cushion. Viral RNA was extracted with a Qiagen kit. Primers were designed to amplify the desired sequences and to contain necessary restriction sites. PCR products were cleaned up with the Qiagen PCR Purification kit and ligated into pGEM-T vector. Plasmids were amplified in E. coli DH5α, purified, and digested with BamHI and Xho1. The insert was purified and recloned into the pET 24b myc His vector.

Recombinant polypeptides were expressed from plasmids in BL21 (DE3)-RP cells. After transformation, cells were spread on LB agar plates containing kanamycin (30 μg/mL) and chloramphenicol (35 μg/mL) and incubated overnight at 37° C. A single colony was picked and grown in 20 mL 2xYT medium containing kanamycin and chloramphenicol as above and grown overnight with shaking at 225 rpm. The 20 mL culture was transferred to 1 liter of 2xYT medium with antibiotics at 30° C. with shaking until the OD₆₀₀ reached 0.3. IPTG was added to 1 mM, and the flask agitated for 4 to 5 hours at 30° C. Two hundred μL of culture was removed for gel analysis.

The remaining culture was centrifuged at 4000×g for 20 minutes at 4° C. to pellet bacteria. The pellet was resuspended in 30 mL of 100 mM NaH₂PO₄, 10 mM Tris HCl, 8 M urea, pH 8.0. PMSF was added to 1 mM, and the mixture was rotated gently at room temperature for 2 hours. The mixture was centrifuged at 10,000×g for 30 minutes at 4° C. to pellet cellular debris. Six mL of a 50% slurry of Ni-NTA agarose (Qiagen, Valencia Calif.) was added to the supernatant containing denatured protein and rotated gently for 1 hour at room temperature. The mixture was then placed in a glass column 1.5 cm ID×40 cm and allowed to drain. The column was washed twice with 20 mL of 100 mM NaH₂PO₄, 10 mM Tris HCl, 8 M urea, pH 6.3. Recombinant protein was eluted with three to four 4-mL aliquots of 100 mM NaH₂PO₄, 10 mM Tris HCl, 8 M urea, pH 5.5-5.7. Proteins were stored at −20° C. until refolding.

Proteins were refolded by adding 231.3 mg of dithiothreitol to the pooled protein solution and stirring gently for 2 hours. Then, the solution was rapidly diluted into 500 mL of refolding buffer (100 mM Tris HCl, pH 8.0, 0.5 M L-arginine, 8 mM oxidized glutathione, 2 mM EDTA, 10 μM pepstatin A, 10 μM leupeptin, 1 mM PMSF) and stirred gently overnight at 4° C. Protein was reconcentrated by tangential flow filtration (Pellicon XL Ultracel PLC 5 kd, Millipore, Bedford Mass.) and dialyzed (Spectra/Por MWCO 3,000) overnight at 4° C. against 10 mM Na phosphate, pH 8.0. Solutions were concentrated by centrifugation (Centriprep, Millipore) at 3,800 rpm for 30 minutes at 4° C. as needed. Proteins were stored in aliquots at −80° C. Protein concentrations and purity were assessed by SDS-polyacrylamide gel electrophoresis.

### Example 9

### Detecting Antibodies to North American and European Genotype PRRS Viruses Using Mixtures of PRRS Virus Polypeptides from Either or Both Genotypes

The following experiments were performed to (1) determine if ELISAs using polypeptides from a North American type PRRS virus (e.g., VR-2332) are capable of detecting PRRS virus positive samples from pigs inoculated with a European type PRRS virus (e.g., Lelystad virus) and (2) determine if ELISAs using polypeptides from a European type PRRS virus (e.g., Lelystad virus) are capable of detecting PRRS virus positive samples from pigs inoculated with a North American type PRRS virus (e.g., VR-2332). The following experiments also were performed to determine if ELISAs using a combination of polypeptides from both North American and European type viruses are capable of detecting PRRS virus positive samples from pigs infected with either type of virus.

The following polypeptides in 15 mM Na₂CO₃, 35 mM NaHCO₃, pH 9.6, were used to coat ELISA plates (COSTAR 3590, 96 well EIA/RIA plate, Corning N.Y.): myc-ORF 7-His (VR-2332), myc-ORF 6 3′-His (VR-2332), myc-ORF 5 3′-His (VR-2332), myc-ORF 7-His (LV), myc-ORF 6 3′-His (LV), and myc-ORF 5 3′-His (LV). These polypeptides were expressed from the respective plasmids containing all or portions of ORF 7 (N), ORF 6 (M), or ORF 5 (GP5). Wells were coated with solutions containing either 100 ng each of the three VR-2332 polypeptides/100 μL, or 100 ng each of the three LV polypeptides/100 μL, or 50 ng each of all six polypeptides/100 μL. Thus, all wells contained 300 ng of polypeptide.

Plates were incubated with coating solution overnight at 4° C. Plate wells were washed one time with PBS, 0.05% Tween 20, pH 7.3-7.5 and blocked with 300 μL of 5% nonfat dry milk in PBS, 0.05% Tween 20, pH 9.4-9.6, per well for 2 hours. Plates were washed 5 times in PBS, 0.05% Tween 20, pH 7.3-7.5, and 100 μL of test serum diluted 1/500 in PBS, 0.05% Tween 20, 5% nonfat dry milk was added to duplicate wells for 1 hour. Plates were washed 5 times, and 100 μL of peroxidase-labeled affinity purified antibody to swine IgG (H+L) (KPL, Gaithersburg Md.) diluted 1/5000 in PBS, 0.05% Tween 20, 5% nonfat dry milk was added for 1 hour. Plates were washed 5 times, and enzyme substrate was added for 5 minutes. Enzyme substrate consisted of 100 μL per well of equal portions of TMB peroxidase substrate solution A and peroxidase H₂O₂ substrate solution B (TMB Peroxidase Substrate System, KPL, Gaithersburg Md.) mixed together. Reactions were stopped with 100 μL of 2 M phosphoric acid, and results were read at 450 nm in a Thermo Max Microplate Reader, Molecular Devices, Sunnyvale Calif.

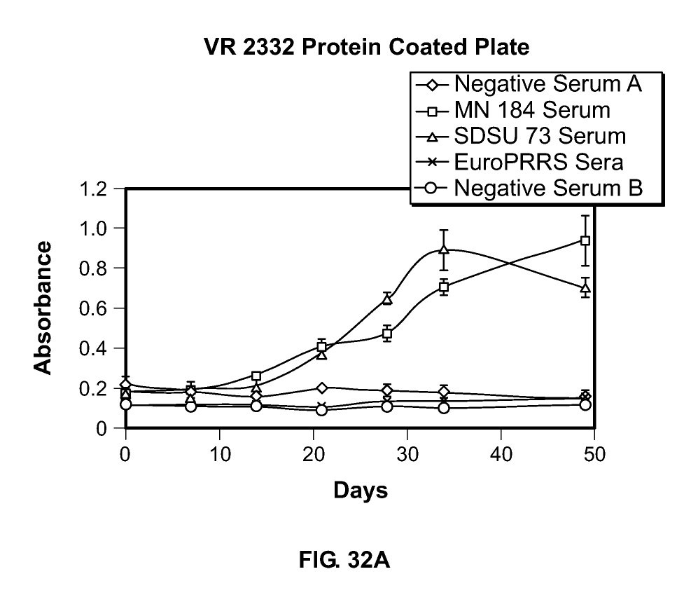

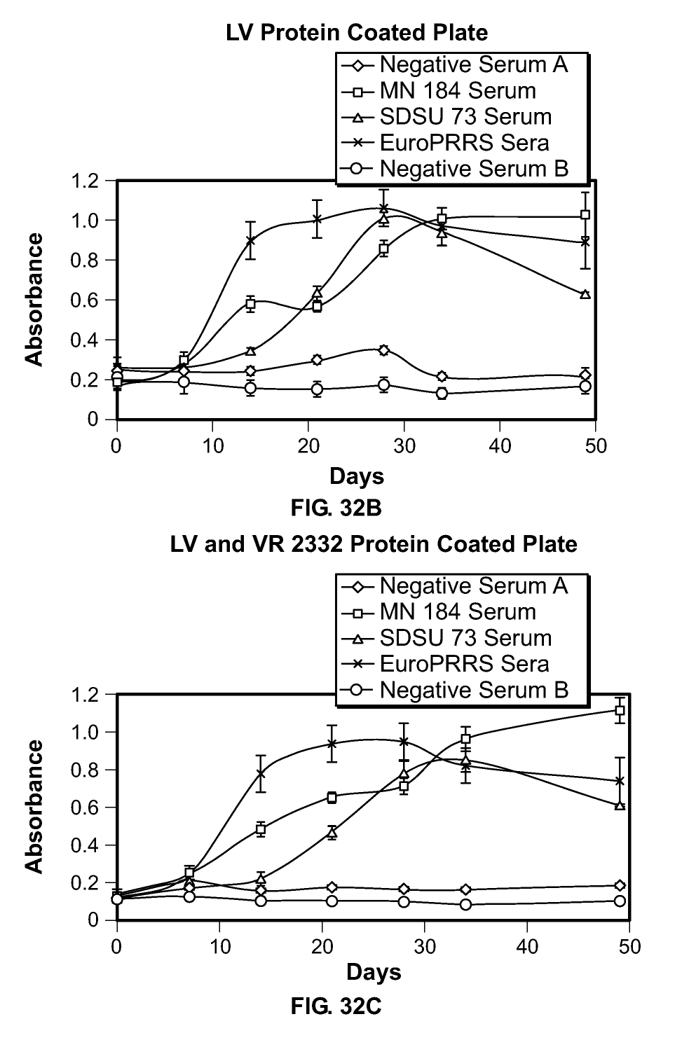

Serum samples were obtained from pigs that were uninfected (negative sera A and negative serum B), infected with a European type PRRS virus (n=9), and either of two North American PRRS virus strains, MN 184 or SDSU 73 (Johnson et al., Vet. Immunol Immunopathol., 102:233-247 (2004)) (n=1 each). Samples were obtained at approximately 0, 7, 14, 21, 28, 35, and 49 days after infection. The mixture of VR-2332 polypeptides detected anti-PRRS virus antibodies in sera of pigs exposed to MN184 or SDSU 73 at 14 days after infection and at all time points thereafter (FIG. 32; panel A). The VR-2332 polypeptides did not detect anti-PRRS virus antibodies in sera of pigs infected with a European PRRS virus.

The mixture of LV polypeptides detected antibodies in sera of pigs exposed to a European genotype PRRS virus at 7 days of infection and at high levels at all time points thereafter (FIG. 32; panel B). The LV polypeptides also detected antibodies in pigs exposed to North American PRRS viruses at day 14 and later (FIG. 32; panel B). The combination of LV and VR-2332 polypeptides detected anti-PRRS virus antibodies in sera of exposed pigs similarly to LV polypeptides alone except that there was a tendency to higher values in the animal exposed to MN 184 (FIG. 32; panel C).

These results indicate that LV polypeptides, such as the mixture of ORF 5 3′, ORF 6 3′, and ORF 7 polypeptides, can detect an antibody response in pigs exposed to European and North American genotype PRRS viruses as early as 7 days after infection. VR-2332 polypeptides specifically recognized sera of pigs exposed to North American type PRRS viruses, and not to sera from pigs exposed to the European type virus. Thus, with the appropriate mixtures of polypeptides, one can detect serological responses to all PRRS viruses and can differentiate between responses to European and North American types.

### Example 10

### Differentiating Between Animals Exposed to a Vaccine or Field Strains of PRRS Virus Using Nonstructural Protein Polypeptides

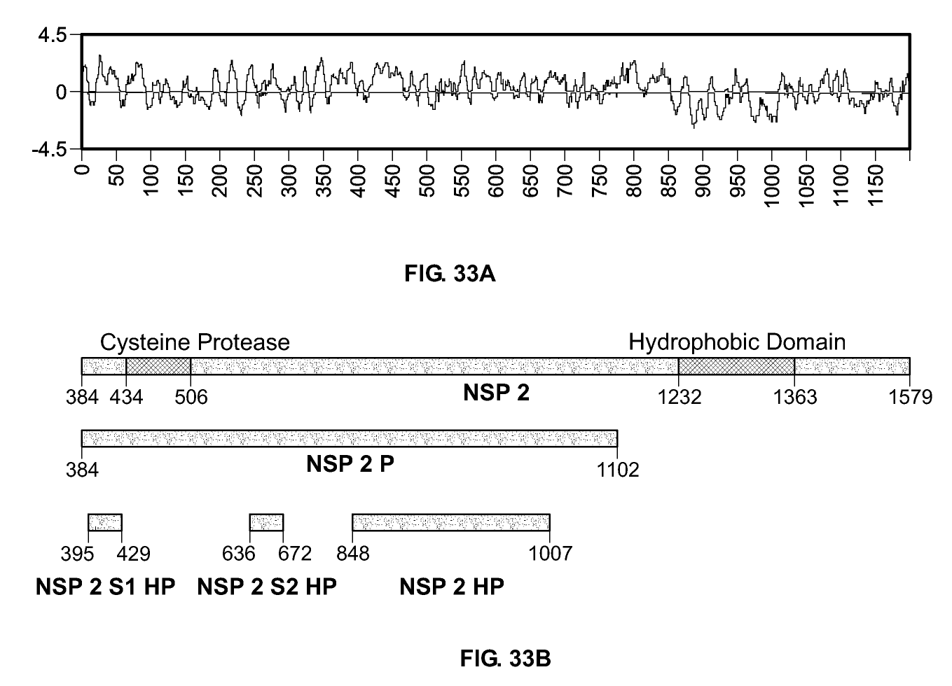

Example 4 demonstrated that swine antibody responses to PRRS viruses can be greater to the nonstructural proteins than to structural proteins such as N. Since detection of NSP's might result in a more sensitive assay, the following experiments were performed to determine if ELISAs comprised of polypeptides derived from a nonstructural protein from VR-2332 or the Ingelvac ATP vaccine strain of PRRS virus would differentiate pigs vaccinated with the Ingelvac MLV or Ingelvac ATP strains from pigs exposed to field strains of PRRS virus. Various polypeptides were produced containing portions of NSP 2 (FIG. 33).

In the first experiment, serum samples from pigs exposed to field viruses or the Ingelvac MLV vaccine virus for 28 days were incubated in microtiter plates coated with myc-NSP 2 P-His (VR-2332) or myc-NSP 2HP-His (VR-2332) polypeptides. Serum from pigs inoculated with MLV vaccine, which was derived from the VR-2332 strain, reacted strongly with both myc-NSP 2P-His and myc-NSP 2 HP-His polypeptides. Positive signals in serum samples diluted 1/1000 from seven pigs ranged from 0.8 to 3.6 OD units against NSP 2P and from 0.7 to 3.1 OD units against NSP 2HP. The relative percent difference (1-(ODNSP 2HP/ODNSP 2P)) after background subtraction, ranged from 9.9 to 23.2 percent. By contrast, serum from pigs inoculated with any of five different wild-type field viruses reacted more strongly with myc-NSP 2P-His polypeptides than with myc-NSP 2HP-His polypeptides. Positive signals among 35 pigs exposed to five different viruses ranged from 0.54 to 3.3 OD units against NSP 2P and from 0.22 to 1.9 OD units against NSP 2HP. The relative percent difference ranged from 36.6 to 85.1 percent.

These results indicate that the NSP 2HP (VR-2332) polypeptide is different from other PRRS virus NSP 2HP polypeptides such that its derived vaccine virus, MLV, elicits antibodies in pigs that react selectively with the NSP 2HP (VR-2332) polypeptide. In addition, the pronounced reactivity of serum samples from field virus-exposed pigs with NSP 2P (VR-2332) indicates that this polypeptide can be used as a diagnostic polypeptide. The relative specificity of the NSP 2 HP polypeptide for the MLV vaccine indicates that a test that compares the relative serological reactivity of a test serum to both VR-2332 NSP 2P and NSP 2HP can differentiate swine vaccinated with Ingelvac MLV from pigs that were exposed to field viruses.

In a second experiment, additional portions from the NSP 2 (VR-2332) polypeptide that showed variation in amino acid sequence among the PRRS virus sequences in GenBank were evaluated for specific reactivity with serum from pigs exposed to Ingelvac MLV vaccine or field viruses as in the previous experiment. ELISA plates were coated with myc-NSP 2P-His (VR-2332) polypeptides or with myc-NSP 2 S1 HP-His (VR-2332) or myc-NSP 2 S2 HP-His (VR-2332) polypeptides.

All pig sera, both from vaccinated and field virus-exposed animals and diluted 1/1000 (n=42), reacted with NSP 2P with high OD values as in the previous experiment. However, the sera reacted weakly with both NSP 2 S1 HP and NSP 2 S2 HP. The OD values of more than 90 percent of the samples were below 0.1. These results indicate that polypeptides of about 30 to 40 amino acids may not encompass sufficient immunoreactivity to discriminate different serological reactivities to a viral infection even though the amino acid sequence variability in the polypeptide is high.

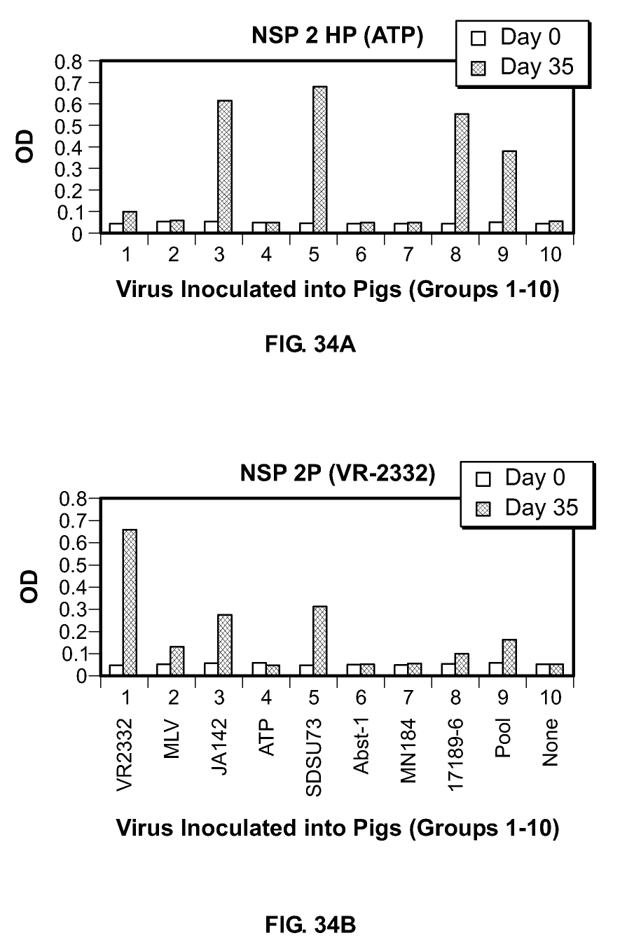

In a third experiment, microtiter plates were coated as described in Example 9 with myc-NSP 2HP-His (ATP) or myc-NSP 2P-His (VR-2332) polypeptides. ELISA assays were performed with serum samples from pigs exposed to vaccine and field viruses as described in Example 4. The assay was performed as described in Example 9 with the exception that serum samples were diluted 1/1000, and color development reactions were stopped after 3 minutes. As shown herein, serum samples from pigs exposed to field viruses or the MLV vaccine react positively with NSP 2P (VR-2332) (FIG. 34). Serum from pigs exposed to Abst-1 or the vaccine strain ATP did not react positively, due to use at doses that elicited a low or negligible immune response. The NSP 2HP (ATP) polypeptide reacted strongly with serum from a pig exposed to JA142, the parental virus of the vaccine, but also to serum from pigs exposed to field viruses SDSU73 and 17198-6. These results demonstrate that the use of nonstructural polypeptides such as NSP 2HP and NSP 2 P from different PRRS virus strains such as Ingelvac ATP and VR-2332 did not result in a serological test that was specific for exposure of swine to a particular strain or isolate. The NSP 2HP (ATP) polypeptide may not be uniquely different from other PRRS virus NSP 2 HP polypeptides in the way that NSP 2HP (VR-2332) is.

### Example 11

### Increasing pH During the Blocking Step Reduces Background Signals

High nonspecific backgrounds in ELISA tests is a commonly encountered problem in swine serology, especially with serum at low dilutions and from older animals such as six months and above. The levels of specific and nonspecific reactivity of positive and negative swine sera were determined on ELISA plates that were blocked at pH 7.4 or 9.6. Wells were coated with equal amounts of N, ORF 5 and ORF 6 ectodomains, and ORF 5 endodomain (all from VR-2332) at zero to 300 ng/well, and positive or negative sera were applied at dilutions of 1/40 to 1/4000. The positive serum samples were obtained from a 7 week-old pig at 3 weeks after exposure to VR-2332. The negative serum samples were obtained from a 6 month-old pig. Blocking was performed with 5% nonfat dry milk in PBS, 0.05% Tween 20 at pH 7.4 or 9.6.

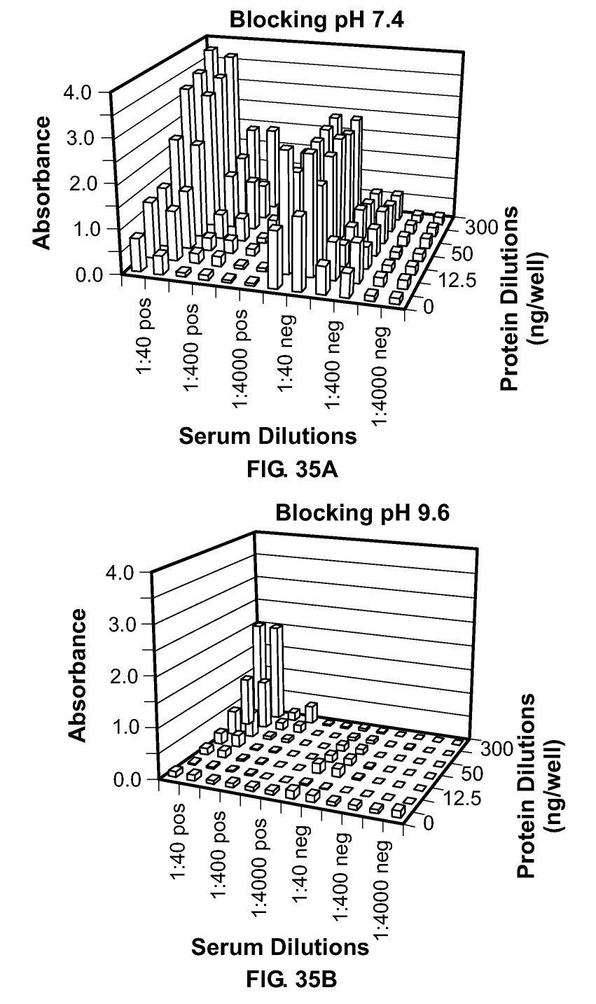

Serum concentration-dependent color development was observed in PRRS virus-positive serum at pH 7.4, but nonspecific color development in PRRS virus-negative serum wells was even higher than specific reactivity at moderate and low levels of antigen on the plate (FIG. 35). At a blocking pH value of 9.6, the nonspecific reactivity was nearly completely abolished. Specific reactivity was reduced by about 50 percent, but the overall signal-to-noise ratio was greatly increased (FIG. 35).

These results indicate that blocking solutions (e.g., protein solutions) applied to ELISA plates at neutral pH do not completely neutralize the protein-binding capacity, resulting in nonspecific binding of swine immunoglobulins and high OD values. The problem is exacerbated at low serum dilutions which would otherwise increase assay sensitivity. Increasing the pH of the blocking solution above 7.4 (e.g., greater than 9) reduced or abolished nonspecific reactivity across a wide range of antigen coating amounts and serum dilutions.

### Example 12

### Producing Recombinant PRRS Virus Polypeptides

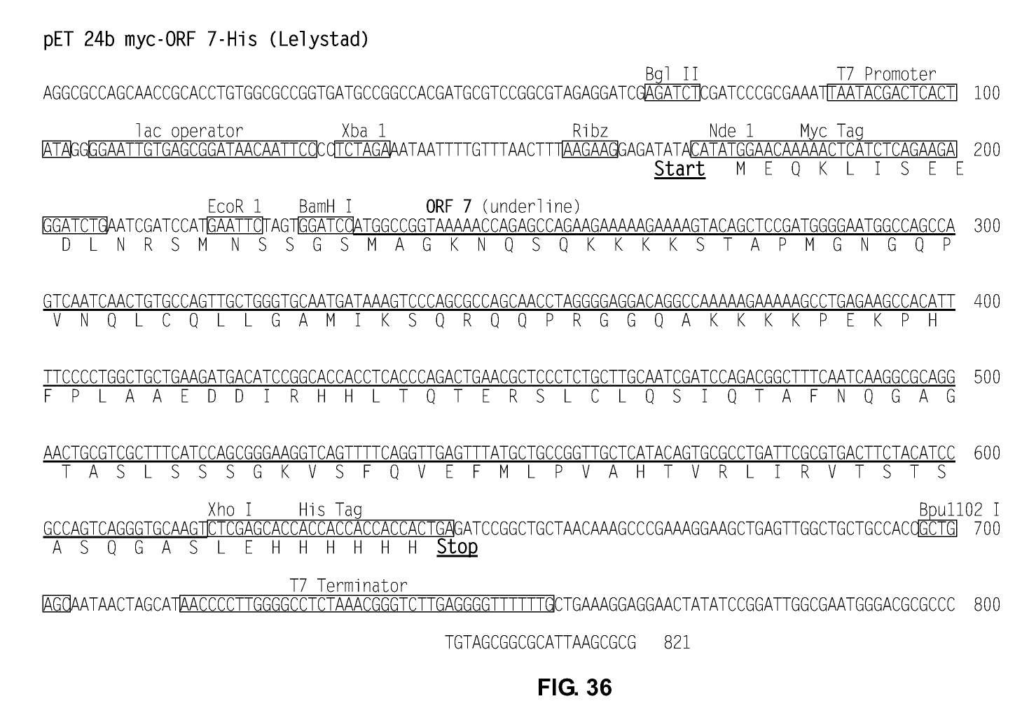

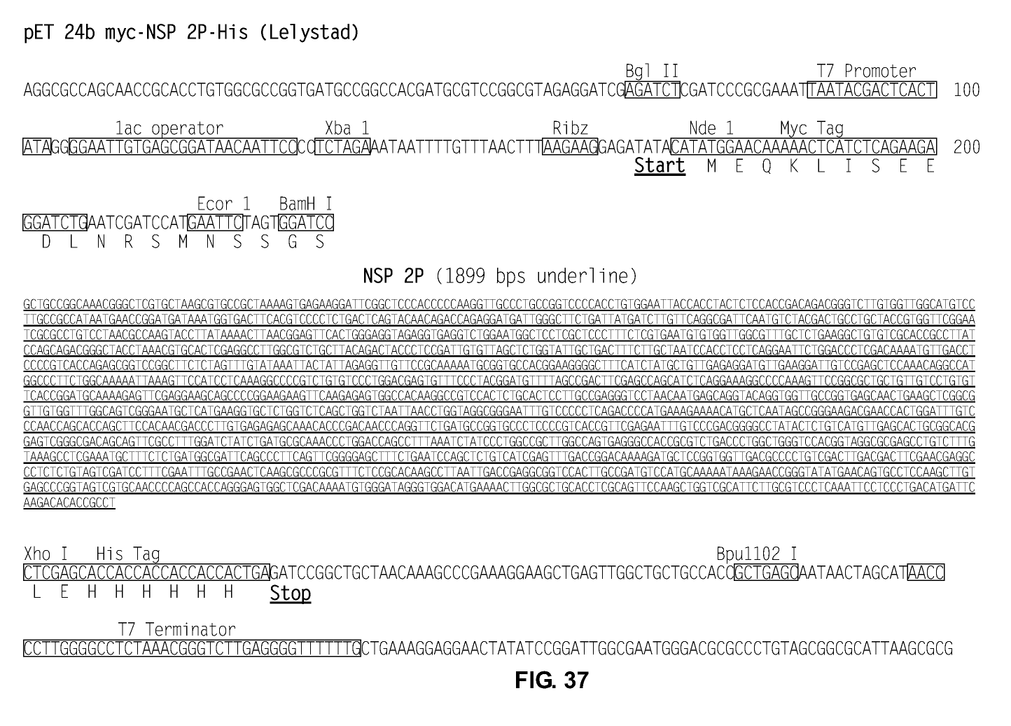

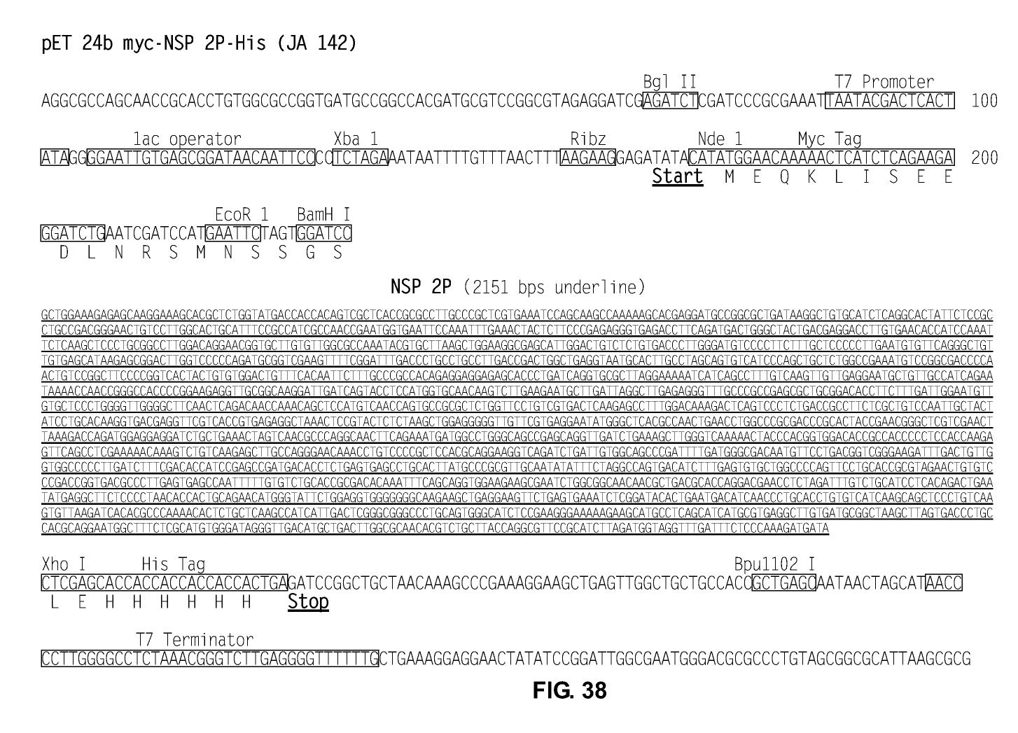

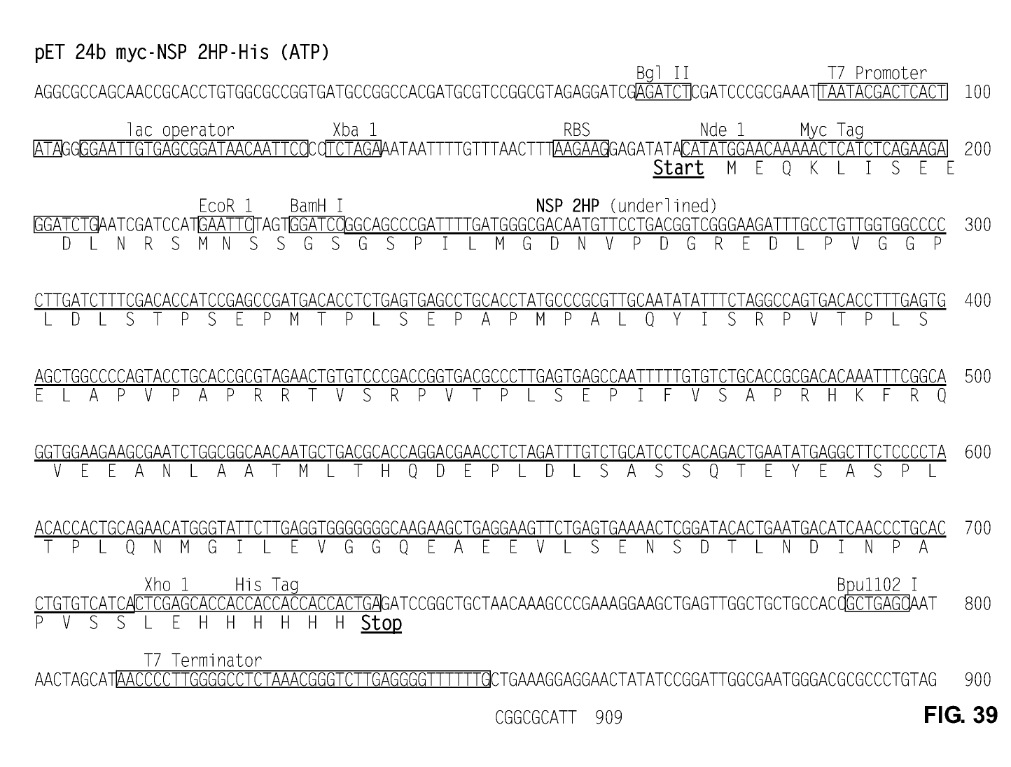

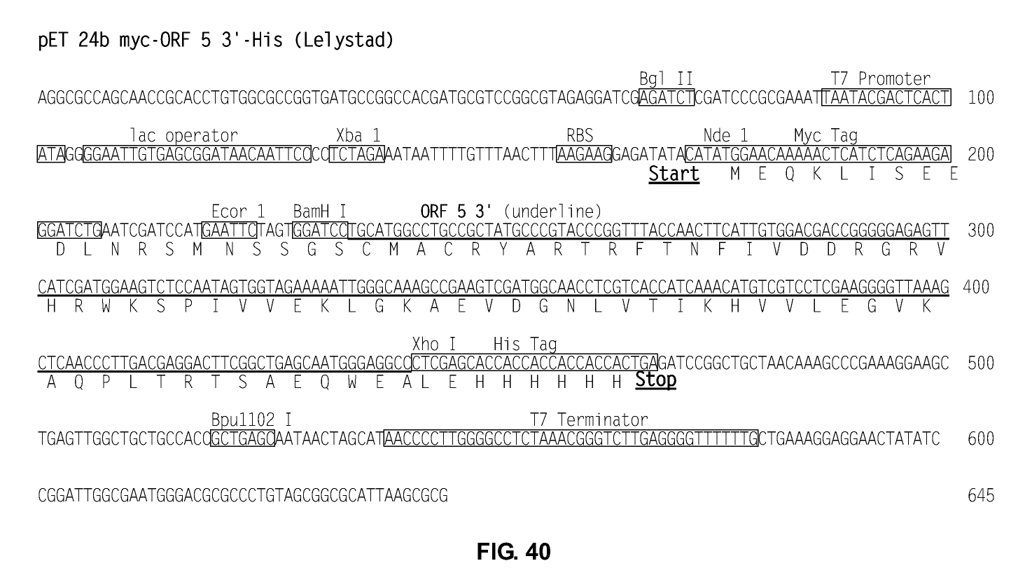

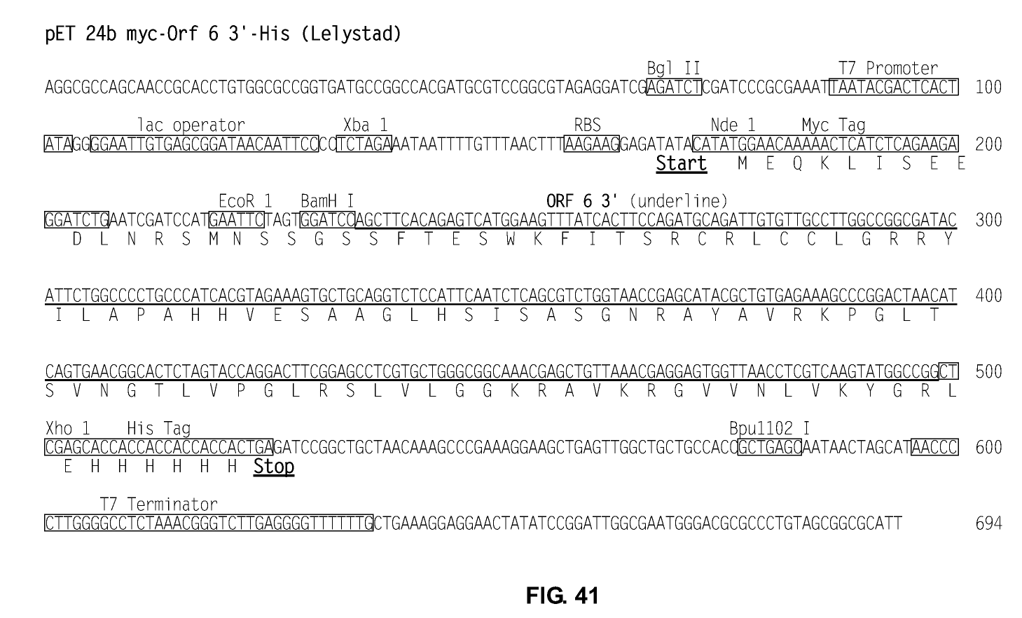

The following polypeptides were produced using methods and materials similar to those described in Example 2: a PRRS virus (Lelystad strain) ORF 7 polypeptide (FIG. 36), a PRRS virus (Lelystad strain) NSP 2P polypeptide (FIG. 37), a PRRS virus (JA 142 strain) NSP 2P polypeptide (FIG. 38), a PRRS virus (ATP strain) NSP 2HP polypeptide (FIG. 39), a PRRS virus (Lelystad strain) endodomain ORF 5 polypeptide (ORF 5 3′; FIG. 40), and a PRRS virus (Lelystad strain) endodomain ORF 6 (ORF 6 3′; FIG. 41) polypeptide.

### Example 13

### Adding Lysozyme to Coating Buffer Increases Reactivity to Nucleocapsid

Lysozyme was included as a control in coating of wells with recombinant polypeptides. Briefly, ELISA wells were coated with 100 ng of polypeptides (e.g., refolded myc-ORF 7-His polypeptide) alone or with various amounts of chicken egg lysozyme (Sigma) in 100 μL carbonate buffer. ELISA was performed with serum samples from two pigs 21 days after infection with PRRS virus strain VR2332 or two uninfected control pig serum.

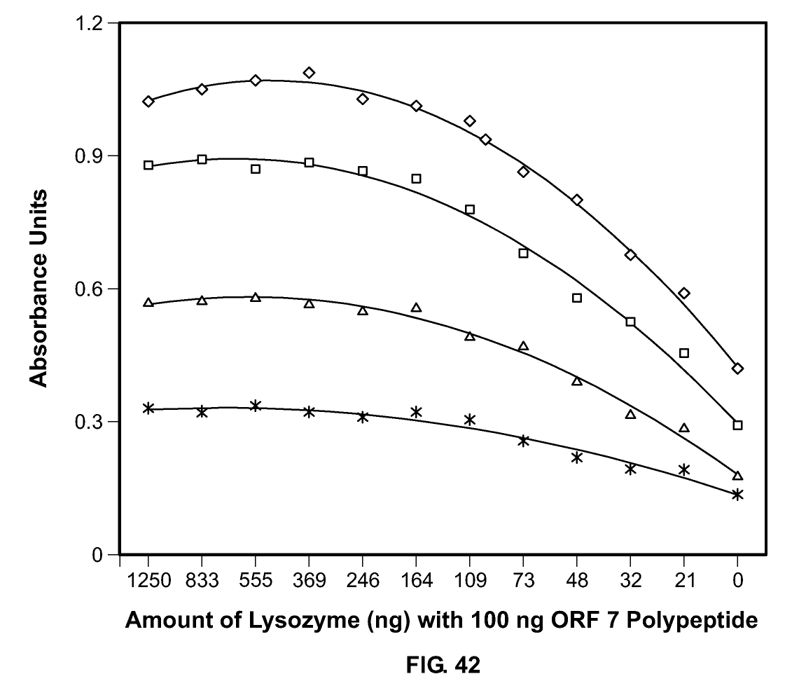

No difference was observed in the intensity of color reactions when plates were coated with various recombinant polypeptides, including myc-ORF 5-3′-His polypeptide and non-refolded myc-ORF 7-His polypeptide, in the presence or absence of lysozyme at various concentrations. Similarly, no reactivity was observed to lysozyme alone. Refolded myc-ORF 7-His polypeptide reactivity, however, was substantially increased in the presence of lysozyme. Moreover, the degree of enhancement was proportional to the amount of lysozyme added (FIG. 42). Inclusion of lysozyme in the coating step increased the specific reactivity of immune PRRS virus serum in a dose-dependent manner up to a maximum of about 200 ng of lysozyme per well (FIG. 42).

The effect was observed at all dilutions of serum examined with greater effects observed with less dilute sera. At a 1/300 dilution of serum, the average specific absorbance was about 0.42 in the absence of lysozyme, but it was greater than 1.0 in the presence of 164 ng or greater of lysozyme. The enhancing effect of lysozyme also was observed with a wide range of coating amounts, including the range of 20 to 500 ng, of refolded myc-ORF 7-His polypeptide per well. About 100 ng of lysozyme per well provided enhanced results under a variety of conditions including, for example, dilution of test serum from 1:40 to 1:5000, incubation of test serum with antigen for 45 minutes to 90 minutes, dilution of second antibody conjugate from 1:500 to 1:5000, and color development reaction time from 2 minutes to 20 minutes. The finding that lysozyme enhances the specific anti-PRRS serological reactivity to the ORF 7 polypeptide is useful since the ORF 7 polypeptide is a major antigen of the PRRS virus and is widely used in serological testing. Conditions that increase the sensitivity of detection of anti-ORF 7 polypeptide antibodies can increase the sensitivity of a diagnostic assay to early infection or exposure to PRRS virus, can increase the duration of detection following exposure or seroconversion, and can provide a basis for a more robust test since the difference between positive and negative results can be increased.

### Example 14

### Increased Stability of Recombinant Viral Protein-Based ELISAs

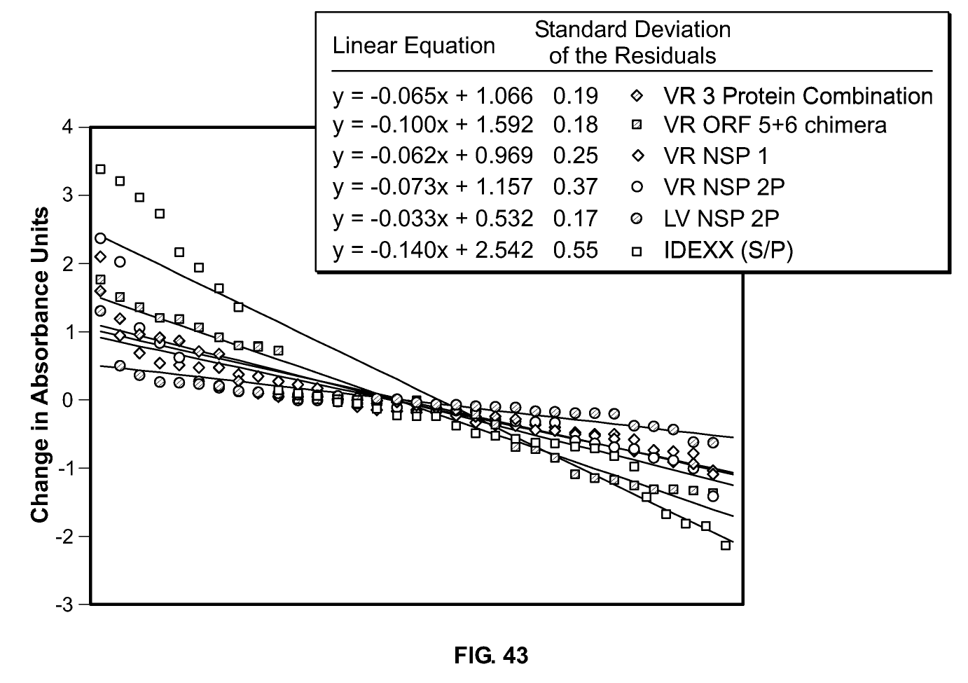

The ability of recombinant polypeptides to detect previous exposure to PRRS virus in pregnant sows in a commercial pig-rearing operation was evaluated in comparison to the IDEXX 2XR ELISA. Sera from 32 pregnant sows in an endemically infected herd were obtained at 35 day intervals and tested for anti-PRRS virus antibodies by IDEXX 2XR ELISA and by ELISA reactivity to a combination of three recombinant PRRS virus polypeptides (ORF 7, ORF 5-3′, and ORF 6-3′, all strain VR2332), or individual PRRS virus polypeptides from strain VR2332 (ORF 5+6 ectodomain chimera, NSP 1, and NSP 2P) or an individual PRRS virus polypeptide from the Lelystad virus strain (NSP 2P; FIG. 37). Briefly, serum samples diluted 1/500 were run in duplicate on ELISA plates coated with 50 ng of ORF 5+6 chimera alone, or 100 ng of a combination of ORF 7, ORF 5-3′ and ORF 6-3′ (33 ng each), or 100 ng of NSP 1, or 100 ng of NSP 2P. The serum samples were also analyzed by IDEXX 2XR ELISA. For each ELISA assay, the difference in average absorbance value (day 35-day 0) was calculated for all 32 pigs and ordered from positive to negative. For each set of data, a hypothetical linear regression equation and regression coefficient was calculated. The resulting values were ordered from highest to lowest value for all 32 animals for each recombinant polypeptide preparation (FIG. 43).

In each instance, a range of values from positive (higher value at interval day 35 than interval day 0) to negative (lower value at interval day 35 than interval day 0) was obtained. The greatest variation (standard deviation of the residuals=0.55) among animals in antibody levels, both positive and negative, occurred in the IDEXX 2XR ELISA test (FIG. 43). The recombinant polypeptide ELISAs exhibited more uniform results in that a hypothetical regression analysis indicated a line with a slope closer to zero, and reduced variation in the highest and lowest responses (FIG. 43). These results were on average more uniform, as indicated by the linear regression equation slope that was closer to zero and the smaller standard deviation of the residuals in each case as compared to IDEXX 2XR ELISA. The most consistent result was obtained with NSP2P derived from Lelystad virus.

Large differences in assay results in a 35 day period can be interpreted as a loss of immunity in animals that exhibit a large decrease in reactivity, and as new infection of susceptible animals in cases of large increases in reactivity. The consequence can be a potential increase in false positive and false negative interpretations of the PRRS virus status of individual animals and of commercial swine populations. The recombinant polypeptide ELISA assays exhibited less variation in animal responses, consistent with exposure of the animals to PRRS virus, the presence of an immune response in the animals, and little change in antibody status of the animals in a 35 day period. These results indicate that the recombinant polypeptide ELISAs, based on individual polypeptides or combinations of polypeptides, can provide a more uniform assessment of herd exposure to PRRS virus and can have a reduced likelihood of false positive and false negative interpretations.

### Example 15

### Detecting Antibodies to North American and European Genotype PRRS Viruses Using Individual PRRS Virus Polypeptides from Either Genotype

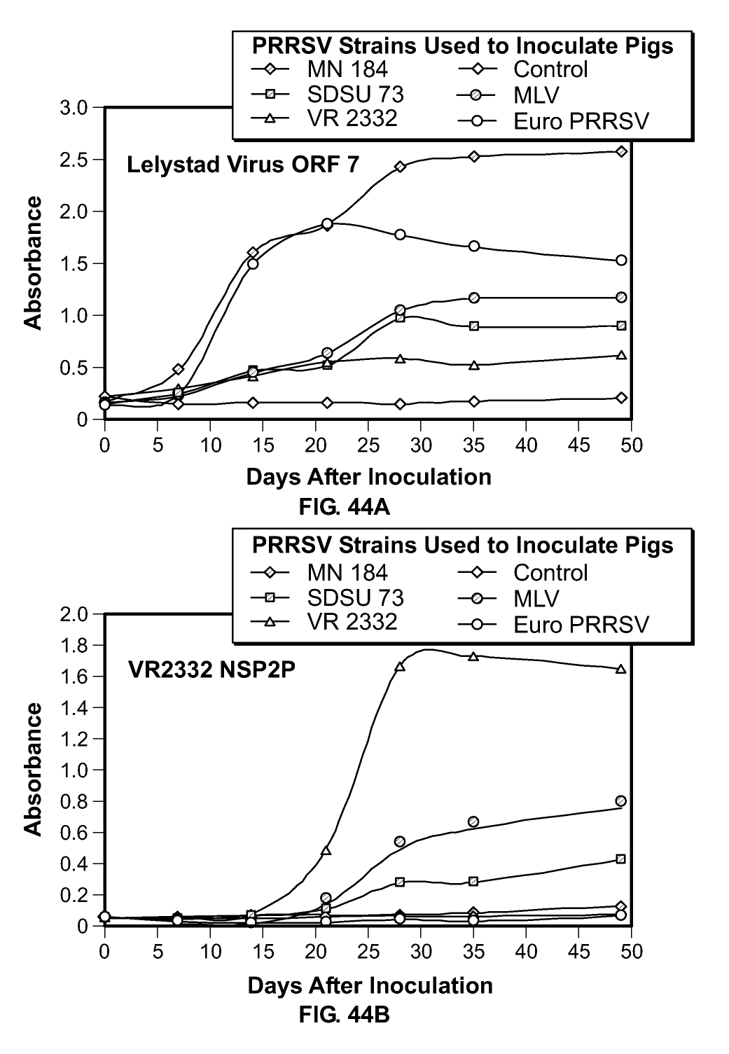

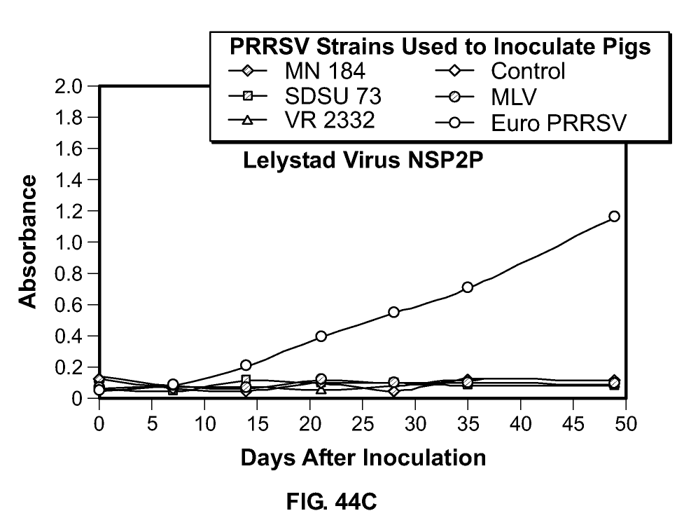

The serum samples described in Example 9 as well as recombinant PRRS virus ORF 7 (from Lelystad virus, a European genotype; FIG. 36) and NSP2P (from VR2332, a North American genotype; or Lelystad virus, FIG. 37) polypeptides were used to examine the specificity of reaction for individual PRRS virus polypeptides. Serum samples from pigs inoculated with a European genotype PRRS virus and with the North American genotype virus, MN 184, reacted strongly with Lelystad virus ORF 7 polypeptides, while North American genotype strains SDSU73, VR2332, and Ingelvac MLV reacted weakly (FIG. 44). These results indicate that ORF 7 polypeptides of Lelystad virus can be used to discriminate serological responses to a subset of North American PRRS viruses as well as to European PRRS virus strains. Serum samples from pigs inoculated with a European genotype PRRS virus reacted exclusively with LV NSP 2P polypeptides and not at all with VR2332 NSP 2P polypeptides (FIG. 44, panels B and C). The VR2332 NSP 2P polypeptide reacted strongly with sera from pigs exposed to VR2332 and positively, but less strongly, with sera of pigs exposed to Ingelvac MLV and SDSU73. It appeared not to react at all with sera from pigs exposed to MN184. These findings indicate that individual PRRS virus polypeptides can detect serological responses to pigs exposed to subsets of PRRS viruses.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A kit for detecting a swine anti-PRRS virus antibody, said kit comprising:
(a) a polypeptide having an amino acid sequence present in a PRRS virus NSP 2 polypeptide and comprising an epitope for said swine anti-PRRS virus antibody; and
(b) an anti-swine Ig antibody.

2. The kit of claim 1, wherein said polypeptide is at least eight amino acid residues in length.

3. The kit of claim 1, wherein said polypeptide comprises an amino acid sequence at least 100 amino acids in length that is at least about 80 percent identical, over said length, to the amino acid sequence encoded by the nucleic acid sequence set forth in SEQ ID NO:5.

4. The kit of claim 1, wherein said polypeptide comprises an amino acid sequence at least 100 amino acids in length that is at least about 90 percent identical, over said length, to the amino acid sequence encoded by the nucleic acid sequence set forth in SEQ ID NO:5.

5. The kit of claim 1, wherein said polypeptide comprises an amino acid sequence encoded by the nucleic acid sequence set forth in SEQ ID NO:11.

6. The kit of claim 1, wherein said polypeptide comprises an amino acid sequence of SEQ ID NO: 39, 45, or 61.

7. The kit of claim 1, wherein said polypeptide is a recombinant polypeptide produced by cells not infected with a PRRS virus.

8. The kit of claim 1, wherein said anti-swine Ig antibody is an anti-swine IgG or IgM antibody.

9. The kit of claim 1, wherein said anti-swine Ig antibody is a goat anti-swine Ig antibody.

10. The kit of claim 1, wherein said kit comprises a polypeptide having an amino acid sequence present in a PRRS virus ORF 5 polypeptide.

11. The kit of claim 1, wherein said anti-swine Ig antibody comprises an enzyme.

12. The kit of claim 1, wherein said kit comprises a control sample containing swine anti-PRRS virus antibody.

13. The kit of claim 1, wherein said kit comprises a control sample containing swine serum lacking swine anti-PRRS virus antibodies.

14. A method for determining whether or not a sample contains a swine anti-PRRS virus antibody, wherein said method comprises:
(a) contacting a polypeptide with said sample under conditions wherein said polypeptide forms a polypeptide:swine anti-PRRS virus antibody complex with an antibody, if present, within said sample, wherein said polypeptide comprises an amino acid sequence present in a PRRS virus NSP 2 polypeptide and comprises an epitope for said swine anti-PRRS virus antibody; and
(b) detecting the presence or absence of said complex, wherein the presence of said complex indicates that said sample contains said swine anti-PRRS virus antibody.

15. The method of claim 14, wherein said sample is a pig serum sample.

16. The method of claim 14, wherein said polypeptide is at least eight amino acid residues in length.

17. The method of claim 14, wherein said polypeptide comprises an amino acid sequence at least 100 amino acids in length that is at least about 80 percent identical, over said length, to the amino acid sequence encoded by the nucleic acid sequence set forth in SEQ ID NO:5.

18. The method of claim 14, wherein said polypeptide comprises the amino acid sequence encoded by the nucleic acid sequence set forth in SEQ ID NO:11.

19. The method of claim 14, wherein said polypeptide comprises an amino acid sequence of SEQ ID NO: 39, 45, or 61.

20. The method of claim 14, wherein said polypeptide is a recombinant polypeptide produced by cells not infected with a PRRS virus.

21. The method of claim 14, wherein said step (b) comprises contacting said complex with an anti-swine Ig antibody.

22. The method of claim 21, wherein said anti-swine Ig antibody contains an enzyme.

23. The method of claim 14, wherein said step (a) comprises contacting said sample with polypeptides within a kit, and wherein said kit comprises a polypeptide having an amino acid sequence present in a PRRS virus ORF 5 polypeptide.
